SEQUENCE LISTING (431 sequences)

SEQ ID NO: 1 - protein, 96 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTCNVAHPASKTKV

SEQ ID NO: 2 - protein, 85 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTC

SEQ ID NO: 3 - protein, 89 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTCNVAH

SEQ ID NO: 4 - protein, 362 aa, synthetic construct
ASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVDNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 5 - protein, 344 aa, synthetic construct
ASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 6 - protein, 358 aa, synthetic construct
ASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVDPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 7 - protein, 358 aa, synthetic construct
ASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 8 - protein, 348 aa, synthetic construct
ASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVDVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 9 - protein, 357 aa, synthetic construct
ASKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECSNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 10 - protein, 342 aa, synthetic construct
ASKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 11 - protein, 353 aa, synthetic construct
ASKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECSPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 12 - protein, 99 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKP

SEQ ID NO: 13 - protein, 101 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPGG

SEQ ID NO: 14 - protein, 100 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPV

SEQ ID NO: 15 - protein, 102 aa, synthetic construct
SSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPK

SEQ ID NO: 16 - protein, 467 aa, synthetic construct
EVQLVESGGDLVAPSQSLSITCTVSGMSLWSNSISWVRQPPGRGLEWLGTIWSQGGTTETDAVKGRFTISRDNAKNTVYLQMNSLRAEDTAMYYCAQIYYYDAEYLHWYFDFWGQGTLVTVSSASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 17 - protein, 471 aa, synthetic construct
EVQLVESGGDLVAPSQSLSITCTVSGMSLWSNSISWVRQPPGRGLEWLGTIWSQGGTTETDAVKGRFTISRDNAKNTVYLQMNSLRAEDTAMYYCAQIYYYDAEYLHWYFDFWGQGTLVTVSSASDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVDVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 18 - protein, 206 aa, synthetic construct
EIVMTQSPASLSASQEEKVTITCRASEGISKNLAWYQQKPGQAPKLLIHATSHLQTGVPSRFSGSGSGTDFTLTISSLQPEDFATYYCQQGYQFPLTFGQGTKVEIKSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKP

SEQ ID NO: 19 - protein, 208 aa, synthetic construct
EIVMTQSPASLSASQEEKVTITCRASEGISKNLAWYQQKPGQAPKLLIHATSHLQTGVPSRFSGSGSGTDFTLTISSLQPEDFATYYCQQGYQFPLTFGQGTKVEIKSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPGG

SEQ ID NO: 20 - protein, 207 aa, synthetic construct
EIVMTQSPASLSASQEEKVTITCRASEGISKNLAWYQQKPGQAPKLLIHATSHLQTGVPSRFSGSGSGTDFTLTISSLQPEDFATYYCQQGYQFPLTFGQGTKVEIKSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPV

SEQ ID NO: 21 - protein, 209 aa, synthetic construct
EIVMTQSPASLSASQEEKVTITCRASEGISKNLAWYQQKPGQAPKLLIHATSHLQTGVPSRFSGSGSGTDFTLTISSLQPEDFATYYCQQGYQFPLTFGQGTKVEIKSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPK

SEQ ID NO: 22 - protein, 450 aa, synthetic construct
EVQLVQSGAEVKKPGASVKVSCKTSGYSFTSSYINWVRQAPGAGLDWMGTINPVSGSTSYAQKFKGRVTLTADTSTSTAYMELSSLRAGDIAVYYCARGGWFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPCREELSKNTVSLWCLIKDFFPPDIDVEWQSNGQQEPESKYRTTPPQLDEDGSYFLYSKLSVDKSRWQRGDTFICYVMHEALHNHYTQESLSHSPGK

SEQ ID NO: 23 - protein, 217 aa, synthetic construct
QSVLTQPASVSGSLGQRVTISCTGSSSDVGSYNYVNWYQQLPGTGPRTLIYGVSKRPSGVPDRFSGSKSGSTATLTISGLQAEDEADYYCGTFAGGSYYGVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 24 - protein, 457 aa, synthetic construct
EVQLVQSGAEVKKPGASVKVSCKTSGYSFTSSYINWVRQAPGAGLDWMGTINPVSGSTSYAQKFKGRVTLTADTSTSTAYMELSSLRAGDIAVYYCARGGWFDYWGQGTLVTVSSASKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPGK

SEQ ID NO: 25 - protein, 210 aa, synthetic construct
QSVLTQPASVSGSLGQRVTISCTGSSSDVGSYNYVNWYQQLPGTGPRTLIYGVSKRPSGVPDRFSGSKSGSTATLTISGLQAEDEADYYCGTFAGGSYYGVFGGGTHLTVLSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKP

SEQ ID NO: 26 - protein, 458 aa, synthetic construct
EVQLVESGGDLVAPSQSLSITCTVSGMSLWSNSISWVRQPPGRGLEWLGTIWSQGGTTETDAVKGRFTISRDNAKNTVYLQMNSLRAEDTAMYYCAQIYYYDAEYLHWYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPCREELSKNTVSLWCLIKDFFPPDIDVEWQSNGQQEPESKYRTTPPQLDEDGSYFLYSKLSVDKSRWQRGDTFICYVMHEALHNHYTQESLSHSPGK

SEQ ID NO: 27 - protein, 217 aa, synthetic construct
EIVMTQSPASLSASQEEKVTITCRASEGISKNLAWYQQKPGQAPKLLIHATSHLQTGVPSRFSGSGSGTDFTLTISSLQPEDFATYYCQQGYQFPLTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 28 - protein, 110 aa, Canis lupus
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 29 - protein, 106 aa, Canis lupus
GQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 30 - protein, 335 aa, synthetic construct
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPCREELSKNTVSLWCLIKDFFPPDIDVEWQSNGQQEPESKYRTTPPQLDEDGSYFLYSKLSVDKSRWQRGDTFICYVMHEALHNHYTQESLSHSPGK

SEQ ID NO: 31 - protein, 14 aa, synthetic construct
NVAHPASKTKVDKP

SEQ ID NO: 32 - protein, 4 aa, synthetic construct
VPKR

SEQ ID NO: 33 - protein, 5 aa, synthetic construct
VPKRE

SEQ ID NO: 34 - protein, 14 aa, Canis lupus
VFNECRCTDTPPCP

SEQ ID NO: 35 - protein, 19 aa, Canis lupus
VPKRENGRVPRPPDCPKCP

SEQ ID NO: 36 - protein, 5 aa, synthetic construct
VFNEC

SEQ ID NO: 37 - protein, 331 aa, Canis lupus
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 38 - protein, 335 aa, Canis lupus
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 39 - protein, 333 aa, Canis lupus
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 40 - protein, 331 aa, Canis lupus
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 41 - protein, 220 aa, Canis lupus
VPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 42 - protein, 219 aa, Canis lupus
APEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 43 - protein, 219 aa, Canis lupus
GCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 44 - protein, 220 aa, Canis lupus
VPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 45 - protein, 18 aa, synthetic construct
PKRENGRVPRPPDCPKCP

SEQ ID NO: 46 - protein, 17 aa, synthetic construct
FNECRCTDTPPCPVPEP

SEQ ID NO: 47 - protein, 22 aa, synthetic construct
PKRENGRVPRPPDCPKCPAPEM

SEQ ID NO: 48 - protein, 20 aa, synthetic construct
AKECECKCNCNNCPCPGCGL

SEQ ID NO: 49 - protein, 17 aa, synthetic construct
PKESTCKCISPCPVPES

SEQ ID NO: 50 - protein, 17 aa, synthetic construct
PKESTCKCIPPCPVPES

SEQ ID NO: 51 - protein, 17 aa, synthetic construct
KTDHPPGPKPCDCPKCP

SEQ ID NO: 52 - protein, 17 aa, synthetic construct
KTASTIESKTGEGPKCP

SEQ ID NO: 53 - protein, 5 aa, synthetic construct
GPGGQ

SEQ ID NO: 54 - protein, 330 aa, Canis lupus [sequence omitted]

SEQ ID NO: 55 - protein, 8 aa, synthetic construct
GMSLWSNS

SEQ ID NO: 56 - protein, 7 aa, synthetic construct
IWSQGGT

SEQ ID NO: 57 - protein, 17 aa, synthetic construct
AQIYYYDAEYLHWYFDF

SEQ ID NO: 58 - protein, 6 aa, synthetic construct
EGISKN

SEQ ID NO: 59 - protein, 9 aa, synthetic construct
QQGYQFPLT

SEQ ID NO: 60 - protein, 123 aa, synthetic construct
EVQLVESGGDLVAPSQSLSITCTVSGMSLWSNSISWVRQPPGRGLEWLGTIWSQGGTTETDAVKGRFTISRDNAKNTVYLQMNSLRAEDTAMYYCAQIYYYDAEYLHWYFDFWGQGTLVTVSS

SEQ ID NO: 61 - protein, 107 aa, synthetic construct
EIVMTQSPASLSASQEEKVTITCRASEGISKNLAWYQQKPGQAPKLLIHATSHLQTGVPSRFSGSGSGTDFTLTISSLQPEDFATYYCQQGYQFPLTFGQGTKVEIK

SEQ ID NO: 62 - protein, 25 aa, synthetic construct
EVQLVESGGDLVAPSQSLSITCTVS

SEQ ID NO: 63 - protein, 17 aa, synthetic construct
ISWVRQPPGRGLEWLGT

SEQ ID NO: 64 - protein, 38 aa, synthetic construct
TETDAVKGRFTISRDNAKNTVYLQMNSLRAEDTAMYYC

SEQ ID NO: 65 - protein, 11 aa, synthetic construct
WGQGTLVTVSS

SEQ ID NO: 66 - protein, 26 aa, synthetic construct
EIVMTQSPASLSASQEEKVTITCRAS

SEQ ID NO: 67 - protein, 17 aa, synthetic construct
LAWYQQKPGQAPKLLIH

SEQ ID NO: 68 - protein, 36 aa, synthetic construct
HLQTGVPSRFSGSGSGTDFTLTISSLQPEDFATYYC

SEQ ID NO: 69 - protein, 10 aa, synthetic construct
FGQGTKVEIK

SEQ ID NO: 70 - protein, 97 aa, Canis lupus
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKP

SEQ ID NO: 71 - protein, 97 aa, Canis lupus
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKP

SEQ ID NO: 72 - protein, 97 aa, Canis lupus
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKP

SEQ ID NO: 73 - protein, 97 aa, Canis lupus
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKP

SEQ ID NO: 74 - protein, 104 aa, Canis lupus
GPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKAR

SEQ ID NO: 75 - protein, 104 aa, Canis lupus
GPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKAR

SEQ ID NO: 76 - protein, 104 aa, Canis lupus
GPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTP

SEQ ID NO: 77 - protein, 104 aa, Canis lupus
GPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKAR

SEQ ID NO: 78 - protein, 106 aa, Canis lupus
KPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 79 - protein, 105 aa, Canis lupus
QPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 80 - protein, 105 aa, Canis lupus
QPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 81 - protein, 106 aa, Canis lupus
QPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 82 - protein, 17 aa, Canis lupus
VAKECECKCNCNNCPCP

SEQ ID NO: 83 - protein, 14 aa, Canis lupus
VPKESTCKCISPCP

SEQ ID NO: 84 - protein, 9 aa, synthetic construct
GDSITSGYW

SEQ ID NO: 85 - protein, 16 aa, synthetic construct
YISYSGITDYNPSLKS

SEQ ID NO: 86 - protein, 12 aa, synthetic construct
ARYGNYGYAMDY

SEQ ID NO: 87 - protein, 15 aa, synthetic construct
RASESVDTYGNSFMH

SEQ ID NO: 88 - protein, 7 aa, synthetic construct
RASNLES

SEQ ID NO: 89 - protein, 9 aa, synthetic construct
QQSYEDPWT

SEQ ID NO: 90 - protein, 9 aa, synthetic construct
GDSITSGYW

SEQ ID NO: 91 - protein, 16 aa, synthetic construct
YISYSGITYYNPSLKS

SEQ ID NO: 92 - protein, 12 aa, synthetic construct
ARYGNYGYAMDY

SEQ ID NO: 93 - protein, 15 aa, synthetic construct
RASESVDTYGNSFIH

SEQ ID NO: 94 - protein, 7 aa, synthetic construct
RASNLES

SEQ ID NO: 95 - protein, 9 aa, synthetic construct
QQSYEDPWT

SEQ ID NO: 96 - protein, 9 aa, synthetic construct
GDSITSGYW

SEQ ID NO: 97 - protein, 16 aa, synthetic construct
YISYSGITDYNPSLKS

SEQ ID NO: 98 - protein, 12 aa, synthetic construct
ARYGNYGYAMDY

SEQ ID NO: 99 - protein, 15 aa, synthetic construct
RASESVDTYGNSFMH

SEQ ID NO: 100 - protein, 7 aa, synthetic construct
RASNLES

SEQ ID NO: 101 - protein, 9 aa, synthetic construct
QQSYEDPWT

SEQ ID NO: 102 - protein, 10 aa, synthetic construct
GYTFTSYVMH

SEQ ID NO: 103 - protein, 17 aa, synthetic construct
YINPKNDGTFYNGKFKG

SEQ ID NO: 104 - protein, 7 aa, synthetic construct
FNYGIAY

SEQ ID NO: 105 - protein, 11 aa, synthetic construct
RASQEISGYLS

SEQ ID NO: 106 - protein, 7 aa, synthetic construct
AASTLDS

SEQ ID NO: 107 - protein, 9 aa, synthetic construct
VQYASYPWT

SEQ ID NO: 108 - protein, 10 aa, synthetic construct
GYTFTSYVMH

SEQ ID NO: 109 - protein, 17 aa, synthetic construct
YINPNNDGTFYNGKFKG

SEQ ID NO: 110 - protein, 7 aa, synthetic construct
FYYGFAY

SEQ ID NO: 111 - protein, 11 aa, synthetic construct
RASQEISGYLS

SEQ ID NO: 112 - protein, 7 aa, synthetic construct
AASTLDS

SEQ ID NO: 113 - protein, 9 aa, synthetic construct
LQYASYPWT

SEQ ID NO: 114 - protein, 13 aa, synthetic construct
TVSGFSLSRYSVH

SEQ ID NO: 115 - protein, 9 aa, synthetic construct
GMIWGGGST

SEQ ID NO: 116 - protein, 9 aa, synthetic construct
TYRGYALDY

SEQ ID NO: 117 - protein, 11 aa, synthetic construct
RASQDISNYLN

SEQ ID NO: 118 - protein, 8 aa, synthetic construct
YYTSRLQS

SEQ ID NO: 119 - protein, 9 aa, synthetic construct
QQANTLPLT

SEQ ID NO: 120 - protein, 13 aa, synthetic construct
AASGFTFSSFGMH

SEQ ID NO: 121 - protein, 11 aa, synthetic construct
AYIRSDSSTIY

SEQ ID NO: 122 - protein, 11 aa, synthetic construct
SGYYGSFSLTY

SEQ ID NO: 123 - protein, 12 aa, synthetic construct
SASSSVSSNYLH

SEQ ID NO: 124 - protein, 8 aa, synthetic construct
YRTSNLPS

SEQ ID NO: 125 - protein, 8 aa, synthetic construct
QQGSGMLT

SEQ ID NO: 126 - protein, 5 aa, synthetic construct
NYYMA

SEQ ID NO: 127 - protein, 17 aa, synthetic construct
SISTGGGNTYYRDSVKG

SEQ ID NO: 128 - protein, 8 aa, synthetic construct
HGTLYFDY

SEQ ID NO: 129 - protein, 13 aa, synthetic construct
ERSSGDIGDSYVS

SEQ ID NO: 130 - protein, 7 aa, synthetic construct
VDDQRPS

SEQ ID NO: 131 - protein, 11 aa, synthetic construct
QSYDSNIDGPV

SEQ ID NO: 132 - protein, 5 aa, synthetic construct
DYYMA

SEQ ID NO: 133 - protein, 17 aa, synthetic construct
TISYDGSSTYYRDSVRG

SEQ ID NO: 134 - protein, 13 aa, synthetic construct
GPLTDWAPNWFAY

SEQ ID NO: 135 - protein, 11 aa, synthetic construct
QTSEDIYSGLA

SEQ ID NO: 136 - protein, 7 aa, synthetic construct
GASRLED

SEQ ID NO: 137 - protein, 9 aa, synthetic construct
QQGLKYPNT

SEQ ID NO: 138 - protein, 5 aa, synthetic construct
SNGVS

SEQ ID NO: 139 - protein, 16 aa, synthetic construct
AISSGGSTYYNSVLKS

SEQ ID NO: 140 - protein, 12 aa, synthetic construct
RLSGYNYVPFAY

SEQ ID NO: 141 - protein, 11 aa, synthetic construct
KASQNIYKHLA

SEQ ID NO: 142 - protein, 7 aa, synthetic construct
NANSLQT

SEQ ID NO: 143 - protein, 8 aa, synthetic construct
QQYYSGDT

SEQ ID NO: 144 - protein, 5 aa, synthetic construct
DYAMT

SEQ ID NO: 145 - protein, 17 aa, synthetic construct
SISGSGGNTYYADSVKG

SEQ ID NO: 146 - protein, 16 aa, synthetic construct
DRLSITIRPRYYGLDV

SEQ ID NO: 147 - protein, 16 aa, synthetic construct
RSSQSLLYSIGYNYLD

SEQ ID NO: 148 - protein, 7 aa, synthetic construct
LGSNRAS

SEQ ID NO: 149 - protein, 9 aa, synthetic construct
MQALQTPYT

SEQ ID NO: 150 - protein, 5 aa, synthetic construct
SYAMS

SEQ ID NO: 151 - protein, 17 aa, synthetic construct
TISDGGSYTHYPDNLMG

SEQ ID NO: 152 - protein, 10 aa, synthetic construct
ESYDGYYVAN

SEQ ID NO: 153 - protein, 11 aa, synthetic construct
RASQSISNNLH

SEQ ID NO: 154 - protein, 7 aa, synthetic construct
YASQSIS

SEQ ID NO: 155 - protein, 9 aa, synthetic construct
QQSNSWPQT

SEQ ID NO: 156 - protein, 5 aa, synthetic construct
DYYMN

SEQ ID NO: 157 - protein, 17 aa, synthetic construct
WIFPGSGATYYNERFMG

SEQ ID NO: 158 - protein, 8 aa, synthetic construct
SDWDVGDF

SEQ ID NO: 159 - protein, 16 aa, synthetic construct
RSSRSLLHTNGITYLS

SEQ ID NO: 160 - protein, 7 aa, synthetic construct
QMSNLAS

SEQ ID NO: 161 - protein, 9 aa, synthetic construct
AQTLGLPRT

SEQ ID NO: 162 - protein, 5 aa, synthetic construct
DYNMD

SEQ ID NO: 163 - protein, 17 aa, synthetic construct
NINPNSESTSYNQKFKG

SEQ ID NO: 164 - protein, 11 aa, synthetic construct
DGNRYDAWFAY

SEQ ID NO: 165 - protein, 10 aa, synthetic construct
SASSSVTYMH

SEQ ID NO: 166 - protein, 7 aa, synthetic construct
STSILAS

SEQ ID NO: 167 - protein, 9 aa, synthetic construct
QQRTSYPLT

SEQ ID NO: 168 - protein, 5 aa, synthetic construct
NYWIH

SEQ ID NO: 169 - protein, 17 aa, synthetic construct
RIDPNSGGTKYNEKFKS

SEQ ID NO: 170 - protein, 9 aa, synthetic construct
FGSTYGFAY

SEQ ID NO: 171 - protein, 17 aa, synthetic construct
KSSQSLLNSRTRKNYLA

SEQ ID NO: 172 - protein, 7 aa, synthetic construct
WASTRES

SEQ ID NO: 173 - protein, 8 aa, synthetic construct
KQSYNLYT

SEQ ID NO: 174 - protein, 10 aa, synthetic construct
GYTFTTYGMS

SEQ ID NO: 175 - protein, 17 aa, synthetic construct
WINIYSGIPTYADDFKG

SEQ ID NO: 176 - protein, 6 aa, synthetic construct
FDGPDY

SEQ ID NO: 177 - protein, 17 aa, synthetic construct
KSSQSLLNSVNQKNYLA

SEQ ID NO: 178 - protein, 7 aa, synthetic construct
FASARVS

SEQ ID NO: 179 - protein, 9 aa, synthetic construct
QQYFSTPLT

SEQ ID NO: 180 - protein, 10 aa, synthetic construct
GFNIKNTYMH

SEQ ID NO: 181 - protein, 17 aa, synthetic construct
RIAPANVDTKYAPKFQG

SEQ ID NO: 182 - protein, 11 aa, synthetic construct
IYYDYDGDIDV

SEQ ID NO: 183 - protein, 11 aa, synthetic construct
HASQNINVWLS

SEQ ID NO: 184 - protein, 7 aa, synthetic construct
KASHLHT

SEQ ID NO: 185 - protein, 9 aa, synthetic construct
QQGQSWPLT

SEQ ID NO: 186 - protein, 10 aa, synthetic construct
GYTFTRYNMH

SEQ ID NO: 187 - protein, 17 aa, synthetic construct
TIYPGYGDTSYNQKFKG

SEQ ID NO: 188 - protein, 13 aa, synthetic construct
EFADDYPIPPFDY

SEQ ID NO: 189 - protein, 16 aa, synthetic construct
RSSQNIVHSNGNTYLE

SEQ ID NO: 190 - protein, 7 aa, synthetic construct
KVSNRFS

SEQ ID NO: 191 - protein, 9 aa, synthetic construct
FQGSHVPYT

SEQ ID NO: 192 - protein, 10 aa, synthetic construct
GFSLTSYGVH

SEQ ID NO: 193 - protein, 17 aa, synthetic construct
WINIYSGIPTYADDFKG

SEQ ID NO: 194 - protein, 6 aa, synthetic construct
FDGPDY

SEQ ID NO: 195 - protein, 17 aa, synthetic construct
KSSQSLLNSVNQKNYLA

SEQ ID NO: 196 - protein, 7 aa, synthetic construct
FASTRVS

SEQ ID NO: 197 - protein, 9 aa, synthetic construct
QQYFSTPLT

SEQ ID NO: 198 - protein, 10 aa, synthetic construct
GYTFTTYGMS

SEQ ID NO: 199 - protein, 17 aa, synthetic construct
WINIYSGIPTYADDFKG

SEQ ID NO: 200 - protein, 6 aa, synthetic construct
FDGPDY

SEQ ID NO: 201 - protein, 17 aa, synthetic construct
KSSQSLLNSVNQKNYLA

SEQ ID NO: 202 - protein, 7 aa, synthetic construct
FASTRVS

SEQ ID NO: 203 - protein, 9 aa, synthetic construct
QQYFSTPLT

SEQ ID NO: 204 - protein, 10 aa, synthetic construct
GFNIKNTYMH

SEQ ID NO: 205 - protein, 17 aa, synthetic construct
RIDPANVNTKYAPKFQG

SEQ ID NO: 206 - protein, 11 aa, synthetic construct
IFYDYDGDIDV

SEQ ID NO: 207 - protein, 11 aa, synthetic construct
HASQNINVWLS

SEQ ID NO: 208 - protein, 7 aa, synthetic construct
KASNLHT

SEQ ID NO: 209 - protein, 9 aa, synthetic construct
QQGQSYPLT

SEQ ID NO: 210 - protein, 10 aa, synthetic construct
GYTFTTYGMS

SEQ ID NO: 211 - protein, 17 aa, synthetic construct
WINIYSGMPTYADDFKG

SEQ ID NO: 212 - protein, 6 aa, synthetic construct
FDGPDY

SEQ ID NO: 213 - protein, 17 aa, synthetic construct
KSSQSLLNSVNQKNYLA

SEQ ID NO: 214 - protein, 7 aa, synthetic construct
FASTRIS

SEQ ID NO: 215 - protein, 9 aa, synthetic construct
QQYFSTPLT

SEQ ID NO: 216 - protein, 5 aa, synthetic construct
NYYMH

SEQ ID NO: 217 - protein, 17 aa, synthetic construct
WINPYTGSAFYAQKFRG

SEQ ID NO: 218 - protein, 12 aa, synthetic construct
EPEKFDSDDSDV

SEQ ID NO: 219 - protein, 14 aa, synthetic construct
TGSSSNIGAGYGVH

SEQ ID NO: 220 - protein, 7 aa, synthetic construct
GDSNRPS

SEQ ID NO: 221 - protein, 11 aa, synthetic construct
QSYDNSLSGYV

SEQ ID NO: 222 - protein, 7 aa, synthetic construct
GFTFSSY

SEQ ID NO: 223 - protein, 6 aa, synthetic construct
NTGGTV

SEQ ID NO: 224 - protein, 11 aa, synthetic construct [sequence omitted]

SEQ ID NO: 225 - protein, 11 aa, synthetic construct
GGDNIGSKSVH

SEQ ID NO: 226 - protein, 7 aa, synthetic construct
YDSRRPT

SEQ ID NO: 227 - protein, 14 aa, synthetic construct
CQVWDNSDRASWVF

SEQ ID NO: 228 - protein, 8 aa, synthetic construct
GGSVTSSH

SEQ ID NO: 229 - protein, 20 aa, synthetic construct
CARGGGYSGTWKDYYVMDYW

SEQ ID NO: 230 - protein, 11 aa, synthetic construct
GGDNLGSKSVH

SEQ ID NO: 231 - protein, 7 aa, synthetic construct
FDTSRPT

SEQ ID NO: 232 - protein, 13 aa, synthetic construct
CQVWDSSAKASVF

SEQ ID NO: 233 - protein, 7 aa, synthetic construct
GFTFSNF

SEQ ID NO: 234 - protein, 6 aa, synthetic construct
NSGGFN

SEQ ID NO: 235 - protein, 17 aa, synthetic construct
CARDWDTHLDTNWFYYW

SEQ ID NO: 236 - protein, 11 aa, synthetic construct
RGDNIGSKSVH

SEQ ID NO: 237 - protein, 7 aa, synthetic construct
YDSSRPT

SEQ ID NO: 238 - protein, 13 aa, synthetic construct
CQVWDISAKASVF

SEQ ID NO: 239 - protein, 7 aa, synthetic construct
GFTFSYY

SEQ ID NO: 240 - protein, 6 aa, synthetic construct
NSGGFS

SEQ ID NO: 241 - protein, 17 aa, synthetic construct
CASESRWGDSYSGMTYW

SEQ ID NO: 242 - protein, 11 aa, synthetic construct
GGDNIGSKSVH

SEQ ID NO: 243 - protein, 7 aa, synthetic construct
YDNSRPT

SEQ ID NO: 244 - protein, 13 aa, synthetic construct
CQVWDSSAKASVF

SEQ ID NO: 245 - protein, 7 aa, synthetic construct
GFTFSNY

SEQ ID NO: 246 - protein, 6 aa, synthetic construct
TSDGIV

SEQ ID NO: 247 - protein, 15 aa, synthetic construct
CASGLFLVVGGGTFW

SEQ ID NO: 248 - protein, 11 aa, synthetic construct
GGDNIGSKSVH

SEQ ID NO: 249 - protein, 7 aa, synthetic construct
SDSSRPT

SEQ ID NO: 250 - protein, 13 aa, synthetic construct
CQVWDSSAKASVF

SEQ ID NO: 251 - protein, 7 aa, synthetic construct
GFTFSSY

SEQ ID NO: 252 - protein, 6 aa, synthetic construct
ASGGTV

SEQ ID NO: 253 - protein, 16 aa, synthetic construct
CTRWKGGTFGYGMDYW

SEQ ID NO: 254 - protein, 11 aa, synthetic construct
GGDNIGSKSVH

SEQ ID NO: 255 - protein, 7 aa, synthetic construct
YDNNRPA

SEQ ID NO: 256 - protein, 13 aa, synthetic construct
CQVWDSSAKASVF

SEQ ID NO: 257 - protein, 7 aa, synthetic construct
GFTFSNY

SEQ ID NO: 258 - protein, 6 aa, synthetic construct
TNDGIV

SEQ ID NO: 259 - protein, 15 aa, synthetic construct
CASGLFLVVGGGTFW

SEQ ID NO: 260 - protein, 11 aa, synthetic construct
GGDNIGSKSVH

SEQ ID NO: 261 - protein, 7 aa, synthetic construct
YDSSRPT

SEQ ID NO: 262 - protein, 13 aa, synthetic construct
CQVWDSSAKASVF

SEQ ID NO: 263 - protein, 7 aa, synthetic construct
GFTFSSN

SEQ ID NO: 264 - protein, 6 aa, synthetic construct
SGDGIY

SEQ ID NO: 265 - protein, 13 aa, synthetic construct
CATGIYPNAFGYW

SEQ ID NO: 266 - protein, 11 aa, synthetic construct
GGDNIGSKSVH

SEQ ID NO: 267 - protein, 7 aa, synthetic construct
YDSSRPT

SEQ ID NO: 268 - protein, 11 aa, synthetic construct
CQVWDSSANVF

SEQ ID NO: 269 - protein, 7 aa, synthetic construct
GFTFSSY

SEQ ID NO: 270 - protein, 6 aa, synthetic construct
SSGGTF

SEQ ID NO: 271 - protein, 15 aa, synthetic construct
CASGLFLVVGGGNYW

SEQ ID NO: 272 - protein, 11 aa, synthetic construct
GGDNIGSKSVH

SEQ ID NO: 273 - protein, 7 aa, synthetic construct
SDSSRPT

SEQ ID NO: 274 - protein, 11 aa, synthetic construct
CQVWDSSASVF

SEQ ID NO: 275 - protein, 7 aa, synthetic construct
GFTFSNY

SEQ ID NO: 276 - protein, 6 aa, synthetic construct
NSDGRV

SEQ ID NO: 277 - protein, 16 aa, synthetic construct
CARWRGGTFGYGMDYW

SEQ ID NO: 278 - protein, 11 aa, synthetic construct
GGDNIGSKSVH

SEQ ID NO: 279 - protein, 7 aa, synthetic construct
YDSSRPT

SEQ ID NO: 280 - protein, 13 aa, synthetic construct
CQVWDSSAKASVF

SEQ ID NO: 281 - protein, 7 aa, synthetic construct
GFTFSNY

SEQ ID NO: 282 - protein, 6 aa, synthetic construct
NSDGRI

SEQ ID NO: 283 - protein, 16 aa, synthetic construct
CARWRGGTFGYGMDYW

SEQ ID NO: 284 - protein, 11 aa, synthetic construct
GGDNIGSKSVH

SEQ ID NO: 285 - protein, 7 aa, synthetic construct
YDSSRPT

SEQ ID NO: 286 - protein, 13 aa, synthetic construct
CQVWDSSAKASVF

SEQ ID NO: 287 - protein, 7 aa, synthetic construct
GFTFHNY

SEQ ID NO: 288 - protein, 6 aa, synthetic construct
SSDGRF

SEQ ID NO: 289 - protein, 15 aa, synthetic construct
CANGLFLVLGGENYW

SEQ ID NO: 290 - protein, 11 aa, synthetic construct
GGDNIGSKSVH

SEQ ID NO: 291 - protein, 7 aa, synthetic construct
YDSSRPT

SEQ ID NO: 292 - protein, 11 aa, synthetic construct
CQVWDSSASVF

SEQ ID NO: 293 - protein, 7 aa, synthetic construct
GFTFSSN

SEQ ID NO: 294 - protein, 6 aa, synthetic construct
SGDGSF

SEQ ID NO: 295 - protein, 13 aa, synthetic construct
CASGIYPNAFGYW

SEQ ID NO: 296 - protein, 11 aa, synthetic construct
GGDNIGSKSVH

SEQ ID NO: 297 - protein, 7 aa, synthetic construct
YDSSRPT

SEQ ID NO: 298 - protein, 11 aa, synthetic construct
CQVRDSSANVF

SEQ ID NO: 299 - protein, 5 aa, synthetic construct
DYGML

SEQ ID NO: 300 - protein, 17 aa, synthetic construct
YISSGSSTIYYADRVKG

SEQ ID NO: 301 - protein, 5 aa, synthetic construct
GTFAY

SEQ ID NO: 302 - protein, 16 aa, synthetic construct
RSSQSLIYNNGNTYLH

SEQ ID NO: 303 - protein, 7 aa, synthetic construct
KVSNRFS

SEQ ID NO: 304 - protein, 9 aa, synthetic construct
SQSTHVPFT

SEQ ID NO: 305 - protein, 5 aa, synthetic construct
DDYMH

SEQ ID NO: 306 - protein, 17 aa, synthetic construct
WIDPENGHTKYASKFQG

SEQ ID NO: 307 - protein, 15 aa, synthetic construct
LRHYYGSSYVSPHYY

SEQ ID NO: 308 - protein, 11 aa, synthetic construct
KASQNVGPNVA

SEQ ID NO: 309 - protein, 7 aa, synthetic construct
SASYRYS

SEQ ID NO: 310 - protein, 9 aa, synthetic construct
QQYNNYPYT

SEQ ID NO: 311 - protein, 5 aa, synthetic construct
DYYMN

SEQ ID NO: 312 - protein, 17 aa, synthetic construct
DINPNNGDTSYNQKFKG

SEQ ID NO: 313 - protein, 14 aa, synthetic construct
GGVLRYPYYYVMDY

SEQ ID NO: 314 - protein, 16 aa, synthetic construct
RSNKSLLHRNGNTYLY

SEQ ID NO: 315 - protein, 7 aa, synthetic construct
RMSNLAS

SEQ ID NO: 316 - protein, 9 aa, synthetic construct
MQHLEFPFT

SEQ ID NO: 317 - protein, 13 aa, synthetic construct
KGSGRTFTSYNMH

SEQ ID NO: 318 - protein, 14 aa, synthetic construct
WVRQMPGKGLEWMG

SEQ ID NO: 319 - protein, 17 aa, synthetic construct
AIYPLTGDTSYNQKSKL

SEQ ID NO: 320 - protein, 10 aa, synthetic construct
RASRSVPYIH

SEQ ID NO: 321 - protein, 8 aa, synthetic construct
YATSALAS

SEQ ID NO: 322 - protein, 9 aa, synthetic construct
QQWLSNPPT

SEQ ID NO: 323 - protein, 5 aa, synthetic construct
NNNVN

SEQ ID NO: 324 - protein, 16 aa, synthetic construct
GVWAGGATDYNSALKS

SEQ ID NO: 325 - protein, 14 aa, synthetic construct
DGGYSSSTLYAMDA

SEQ ID NO: 326 - protein, 11 aa, synthetic construct
RASEDIYNALA

SEQ ID NO: 327 - protein, 7 aa, synthetic construct
NTDTLHT

SEQ ID NO: 328 - protein, 9 aa, synthetic construct
QHYFHYPRT

SEQ ID NO: 329 - protein, 7 aa, synthetic construct
LIGYDIN

SEQ ID NO: 330 - protein, 16 aa, synthetic construct
MIWGDGTTDYNSALKS

SEQ ID NO: 331 - protein, 13 aa, synthetic construct
GGYYYGTSYYFDY

SEQ ID NO: 332 - protein, 11 aa, synthetic construct
RASQDISNHLN

SEQ ID NO: 333 - protein, 7 aa, synthetic construct
YISRFHS

SEQ ID NO: 334 - protein, 9 aa, synthetic construct
QQSKTLPYT

SEQ ID NO: 335 - protein, 8 aa, synthetic construct
TNNIGILG

SEQ ID NO: 336 - protein, 11 aa, synthetic construct
QSFDTTLGAHV

SEQ ID NO: 337 - protein, 8 aa, synthetic construct
GFTFSSHG

SEQ ID NO: 338 - protein, 8 aa, synthetic construct
INSGGSST

SEQ ID NO: 339 - protein, 19 aa, synthetic construct
AKESVGGVVEQLVGPHFDY

SEQ ID NO: 340 - protein, 8 aa, synthetic construct
TMDIDIFG

SEQ ID NO: 341 - protein, 12 aa, synthetic construct
QSGDSTLGALAI

SEQ ID NO: 342 - protein, 8 aa, synthetic construct
GFTFSTYG

SEQ ID NO: 343 - protein, 8 aa, synthetic construct
ISSGGSST

SEQ ID NO: 344 - protein, 17 aa, synthetic construct
AGSRYTYAYGGGYEFHF

SEQ ID NO: 345 - protein, 5 aa, synthetic construct
SSWMN

SEQ ID NO: 346 - protein, 17 aa, synthetic construct
QIYPGDGDTNYNGKFKG

SEQ ID NO: 347 - protein, 10 aa, synthetic construct
ARHYDGSTDY

SEQ ID NO: 348 - protein, 11 aa, synthetic construct
RASENIYSNLA

SEQ ID NO: 349 - protein, 7 aa, synthetic construct
AATNLAD

SEQ ID NO: 350 - protein, 9 aa, synthetic construct
QHFWGTPYT

SEQ ID NO: 351 - protein, 7 aa, synthetic construct
FSSYGMH

SEQ ID NO: 352 - protein, 11 aa, synthetic construct
VISYDGSIKYY

SEQ ID NO: 353 - protein, 14 aa, synthetic construct
TGEYSGYDTDPQYS

SEQ ID NO: 354 - protein, 11 aa, synthetic construct
RASQGIGDDLG

SEQ ID NO: 355 - protein, 7 aa, synthetic construct
GTSTLQS

SEQ ID NO: 356 - protein, 9 aa, synthetic construct
LQDSNYPLT

SEQ ID NO: 357 - protein, 7 aa, synthetic construct
GYIFITY

SEQ ID NO: 358 - protein, 6 aa, synthetic construct
FPASGS

SEQ ID NO: 359 - protein, 12 aa, synthetic construct
GDGNYALDAMDY

SEQ ID NO: 360 - protein, 15 aa, synthetic construct
RASESVDSYGNSFMH

SEQ ID NO: 361 - protein, 7 aa, synthetic construct
LASNLES

SEQ ID NO: 362 - protein, 9 aa, synthetic construct
QQNNEDPLT

SEQ ID NO: 363 - protein, 8 aa, synthetic construct
GFTLTQYG

SEQ ID NO: 364 - protein, 9 aa, synthetic construct
VIWATGATD

SEQ ID NO: 365 - protein, 13 aa, synthetic construct
DGWWYATSWYFDV

SEQ ID NO: 366 - protein, 11 aa, synthetic construct
KASQDINHYLN

SEQ ID NO: 367 - protein, 7 aa, synthetic construct
YTSRLHS

SEQ ID NO: 368 - protein, 9 aa, synthetic construct
QQGDHFPRT

SEQ ID NO: 369 - protein, 5 aa, synthetic construct
SYAMS

SEQ ID NO: 370 - protein, 17 aa, synthetic construct
YISSGGDYIYYADTVKG

SEQ ID NO: 371 - protein, 9 aa, synthetic construct
DPITGTFAY

SEQ ID NO: 372 - protein, 11 aa, synthetic construct
RASQDINNYLN

SEQ ID NO: 373 - protein, 7 aa, synthetic construct
YTSTLHS

SEQ ID NO: 374 - protein, 9 aa, synthetic construct
QQGNTLPWT

SEQ ID NO: 375 - protein, 5 aa, synthetic construct
SYAMS

SEQ ID NO: 376 - protein, 17 aa, synthetic construct
YISSGGDYFYYADTVKG

SEQ ID NO: 377 - protein, 9 aa, synthetic construct
DPITGTFAY

SEQ ID NO: 378 - protein, 11 aa, synthetic construct
RASQDITNYLN

SEQ ID NO: 379 - protein, 7 aa, synthetic construct
YTSTLHS

SEQ ID NO: 380 - protein, 9 aa, synthetic construct
QQGHMLPWT

SEQ ID NO: 381 - protein, 5 aa, synthetic construct
NYWMN

SEQ ID NO: 382 - protein, 17 aa, synthetic construct
QIYPGHVNTNYNGNFKD

SEQ ID NO: 383 - protein, 12 aa, synthetic construct
SADNSGFVLFAY

SEQ ID NO: 384 - protein, 15 aa, synthetic construct
RASKSVSTSGYSYLH

SEQ ID NO: 385 - protein, 7 aa, synthetic construct
LASNLES

SEQ ID NO: 386 - protein, 9 aa, synthetic construct
QHSRELPLT

SEQ ID NO: 387 - protein, 5 aa, synthetic construct
DYYMA

SEQ ID NO: 388 - protein, 17 aa, synthetic construct
NINYDGSSTYYLDSLKS

SEQ ID NO: 389 - protein, 8 aa, synthetic construct
GLTWDFDV

SEQ ID NO: 390 - protein, 11 aa, synthetic construct
KASQDVDTAVA

SEQ ID NO: 391 - protein, 7 aa, synthetic construct
LASTRHT

SEQ ID NO: 392 - protein, 9 aa, synthetic construct
QQYSRFPLT

SEQ ID NO: 393 - protein, 4 aa, synthetic construct
GGGS

SEQ ID NO: 394 - protein, 4 aa, synthetic construct
SGGG

SEQ ID NO: 395 - protein, 5 aa, synthetic construct
GGGGS

SEQ ID NO: 396 - protein, 5 aa, synthetic construct
SGGGG

SEQ ID NO: 397 - protein, 6 aa, synthetic construct
GGGGGS

SEQ ID NO: 398 - protein, 6 aa, synthetic construct
SGGGGG

SEQ ID NO: 399 - protein, 7 aa, synthetic construct
GGGGGGS

SEQ ID NO: 400 - protein, 7 aa, synthetic construct
SGGGGGG

SEQ ID NO: 401 - protein, 8 aa, synthetic construct
GYSFTSSY

SEQ ID NO: 402 - protein, 8 aa, synthetic construct
INPVSGST

SEQ ID NO: 403 - protein, 8 aa, synthetic construct
ARGGWFDY

SEQ ID NO: 404 - protein, 9 aa, synthetic construct
SSDVGSYNY

SEQ ID NO: 405 - protein, 11 aa, synthetic construct
GTFAGGSYYGV

SEQ ID NO: 406 - protein, 213 aa, synthetic construct
QSALTQPSSVSGTLGQTVTISCDGSSSDVGSYNYVNWYQQFPGTSPKLLIYGVSRRPSGIPARFSGSKSGNTASLTISGLQAEDEADYYCGTFAGGSYYGVFGGGTHLTVLSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPK

SEQ ID NO: 407 - protein, 456 aa, synthetic construct
EVQLVESGGDLVKPGGSLRLSCVASGYSFTSTYIQWVRQAPGKGLQWMGTINPVSGSTLYADAVKGRFTISRDNARNTVYLQMNSLRAEDTAVYYCARSGWFDYWGQGTLVTVSSASKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNHYTQESLSHSPG

SEQ ID NO: 408 - protein, 452 aa, synthetic construct
EVQLVESGGDLVKPGGSLRLSCVASGFTFSNYDMAWVRQAPGKGLQWVASMSPGGGSIYYRDAVKGRFTISRDNARNTVYLQMNSLRAEDTAVYYCAREGELGPFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPCREELSKNTVSLWCLIKDFFPPDIDVEWQSNGQQEPESKYRTTPPQLDEDGSYFLYSKLSVDKSRWQRGDTFICYVMHEALHNHYTQESLSHSPG

SEQ ID NO: 409 - protein, 217 aa, synthetic construct
EIVMTQSPASLSLSQEEKVTITCRASQSVGINVDWYQQKPGQAPKLLIYGASNRHTGVPSRFSGSGSGTDFTFTISSLEPEDVAVYYCLQYGSIPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 410 - protein, 105 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 411 - protein, 334 aa, synthetic construct
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 412 - protein, 4 aa, synthetic construct
TVSW

SEQ ID NO: 413 - protein, 11 aa, synthetic construct
NVAHPASKTKV

SEQ ID NO: 414 - protein, 9 aa, synthetic construct
FTFDDYAMH

SEQ ID NO: 415 - protein, 18 aa, synthetic construct
AITWNSGHIDYADSVEGR

SEQ ID NO: 416 - protein, 14 aa, synthetic construct
AKVSYLSTASSLDY

SEQ ID NO: 417 - protein, 11 aa, synthetic construct
RASQGIRNYLA

SEQ ID NO: 418 - protein, 6 aa, synthetic construct
AASTLQ

SEQ ID NO: 419 - protein, 9 aa, synthetic construct
QRYNRAPYT

SEQ ID NO: 420 - protein, 5 aa, synthetic construct
NHWMN

SEQ ID NO: 421 - protein, 19 aa, synthetic construct
EIRSKSINSATHYAESVKG

SEQ ID NO: 422 - protein, 9 aa, synthetic construct
NYYGSTYDY

SEQ ID NO: 423 - protein, 11 aa, synthetic construct
RASQFVGSSIH

SEQ ID NO: 424 - protein, 7 aa, synthetic construct
YASESMS

SEQ ID NO: 425 - protein, 9 aa, synthetic construct
QQSHSWPFT

SEQ ID NO: 426 - protein, 13 aa, synthetic construct
VASGFSLTNNNVN

SEQ ID NO: 427 - protein, 9 aa, synthetic construct
GVWAGGATD

SEQ ID NO: 428 - protein, 16 aa, synthetic construct
ARDGGYSSSTLYAMDA

SEQ ID NO: 429 - protein, 11 aa, synthetic construct
RASEDIYNALA

SEQ ID NO: 430 - protein, 8 aa, synthetic construct
YNTDTLHT

SEQ ID NO: 431 - protein, 9 aa, synthetic construct
QHYFGYPRT

(19) United States
(12) Patent Application Publication
(10) Pub. No.: US 2026/0297171 A1
(43) Pub. Date: Oct. 1, 2026

(54) ANTIBODIES FOR USE IN CANINES

(71) Applicant: Invetx, Inc., Boston, MA (US)

(72) Inventors: Leila SEVIGNY, Hopkinton, MA (US); William BRONDYK, Brewster, MA (US); Shivaanee KOTHAVALE, Watertown, MA (US)

(21) Appl. No.: 19/573,119

(22) Filed: Mar. 20, 2026

Related U.S. Application Data
(60) Provisional application No. 63/775,681, filed on Mar. 21, 2025.

(51) Int. Cl. C07K 16/22; C07K 16/28

(52) U.S. Cl. CPC C07K 16/22; C07K 16/2863; C07K 2317/20; C07K 2317/31; C07K 2317/522; C07K 2317/53; C07K 2317/565; C07K 2317/66; C07K 2317/92; C07K 2317/94

(57) ABSTRACT

The present disclosure features antibodies (e.g., bispecific antibodies) for use in canines, pharmaceutical compositions comprising such antibodies, and methods of use thereof.

### SEQUENCE LISTING

The instant application contains a Sequence Listing which has been submitted electronically in XML format and is hereby incorporated by reference in its entirety. Said XML copy, created on Mar. 17, 2026, is named “51682-019002_Sequence_Listing_3_17_26” and is 387,576 bytes in size.

### FIELD OF THE INVENTION

This disclosure relates generally to antibodies (e.g., bispecific antibodies) for use in canines, pharmaceutical compositions comprising such antibodies, and methods of use thereof.

### BACKGROUND OF THE INVENTION

Bispecific antibodies are capable of binding two or more antigens and have broad therapeutic applications. Bispecific antibodies may include two binding domains, which may be directed against two different antigens or two different epitopes on the same antigen. A variety of bispecific antibody formats have been developed for use in humans, such as tetravalent bispecific antibodies by the fusion of, e.g., an IgG antibody and single chain domains, and other formats of bispecific antibodies in which the antibody core structure (e.g., IgA, IgD, IgE, IgG, or IgM) is no longer retained. For example, dia-, tria-, and tetrabodies, minibodies, and several single chain formats (scFv, Bis-scFv) capable of binding two or more antigens have been developed. Such formats may use linkers either to fuse the antibody core (e.g., IgA, IgD, IgE, IgG or IgM) to a binding protein (e.g., scFv) or to fuse, e.g., two Fab fragments or scFvs. Production of bispecific antibodies can be difficult. For example, due to the presence of mispaired byproducts, sophisticated purification procedures may be required to produce bispecific antibodies.

Although a number of bispecific antibodies have been approved or are in clinical development for the treatment of humans, at present, there are no approved bispecific antibodies for use in dogs. Additionally, there is limited guidance in the art regarding approaches to successfully produce bispecific antibodies based on canine antibodies. In particular, it remains challenging to increase the overall yield, homogeneity, and stability of bispecific antibodies.

Accordingly, there is a need in the art for bispecific antibodies that can be used for the treatment or prevention of canine diseases or disorders.

### SUMMARY OF THE INVENTION

Provided herein are, inter alia, antibodies (e.g., bispecific antibodies) for use in canines, nucleic acids encoding such antibodies, vectors, host cells, methods of production, pharmaceutical compositions containing such antibodies, and methods of use thereof.

In a first aspect, the disclosure features a bispecific antibody that includes:
(a) a first antigen-binding moiety that binds to a first antigen, in which the first antigen-binding moiety includes: (i) a first light chain including a first light chain variable region (VL) and a first light chain constant domain (CL); and (ii) a first heavy chain including a first heavy chain variable region (VH), a first heavy chain constant domain 1 (CH1), and a first Fc subunit; and
(b) a second antigen-binding moiety that binds to a second antigen, in which the second antigen-binding moiety includes: (i) a second light chain including a second VL and a second CH1; and (ii) a second heavy chain including a second VH, a second CL, and a second Fc subunit, and
in which the first CH1 and the second CH1 are derived from a CH1 of a canine IgG, the first CL and the second CL are derived from a CL of a canine IgG, and the first Fc subunit and the second Fc subunit are derived from an Fc subunit of a canine IgG.

In some embodiments, the second CH1 includes a sequence having at least 80% identity (e.g., 85%, 90%, 95%, 97%, 99%, or 100% identity) to: (a) a continuous sequence of amino acid residues within SEQ ID NO: 37 that includes at least amino acids 1-83 of SEQ ID NO: 37; (b) a continuous sequence of amino acid residues within SEQ ID NO: 38 that includes at least amino acids 1-83 of SEQ ID NO: 38; (c) a continuous sequence of amino acid residues within SEQ ID NO: 39 that includes at least amino acids 1-X of SEQ ID NO: 39; or (d) a continuous sequence of amino acid residues within SEQ ID NO: 40 that includes at least amino acids 1-X of SEQ ID NO: 40.

In some embodiments, the second CH1 includes a sequence containing: (a) a continuous sequence of amino acid residues within SEQ ID NO: 37 that includes at least amino acids 1-83 of SEQ ID NO: 37; (b) a continuous sequence of amino acid residues within SEQ ID NO: 38 that includes at least amino acids 1-83 of SEQ ID NO: 38; (c) a continuous sequence of amino acid residues within SEQ ID NO: 39 that includes at least amino acids 1-X of SEQ ID NO: 39; or (d) a continuous sequence of amino acid residues within SEQ ID NO: 40 that includes at least amino acids 1-X of SEQ ID NO: 40.

In some embodiments, the second CH1 includes a sequence having at least 80% identity (e.g., 85%, 90%, 95%, 97%, 99%, or 100% identity) to: (a) a continuous sequence of amino acid residues within SEQ ID NO: 37 that includes at least amino acids 1-97 of SEQ ID NO: 37; or (b) a continuous sequence of amino acid residues within SEQ ID NO: 38 that includes at least amino acids 1-102 of SEQ ID NO: 38.

In some embodiments, the second CH1 includes a sequence containing: (a) a continuous sequence of amino acid residues within SEQ ID NO: 37 that includes at least amino acids 1-97 of SEQ ID NO: 37; or (b) a continuous sequence of amino acid residues within SEQ ID NO: 38 that includes at least amino acids 1-102 of SEQ ID NO: 38.

In some embodiments, the second CH1 further includes a serine-serine linker at the N-terminus.

In some embodiments, the second CH1 further includes one or more (e.g., one, two, three, four, five, six, seven, eight, nine, ten, eleven, twelve, or more) glycine residues at the C-terminus.

In some embodiments, the second CL includes a sequence having at least 80% identity (e.g., 85%, 90%, 95%, 97%, 99%, or 100% identity) to: (a) a continuous sequence of amino acid residues within SEQ ID NO: 28 that includes at least amino acids 3-106 of SEQ ID NO: 28; or (b) a continuous sequence of amino acid residues within SEQ ID NO: 29 that includes at least amino acids 4-105 of SEQ ID NO: 29.

In some embodiments, the second CL includes a sequence containing: (a) a continuous sequence of amino acid residues within SEQ ID NO: 28 that includes at least amino acids 3-106 of SEQ ID NO: 28; or (b) a continuous sequence of amino acid residues within SEQ ID NO: 29 that includes at least amino acids 4-105 of SEQ ID NO: 29.

In some embodiments, the second CL further includes an alanine-serine linker at the N-terminus.

In some embodiments, the second CL further includes one or more (e.g., one, two, three, four, five, six, seven, eight, nine, ten, eleven, twelve, or more) continuous or discontinuous amino acid residues within the sequence of SEQ ID NO: 31. In some embodiments, the second CL further includes the sequence of SEQ ID NO: 31.

In some embodiments, the first Fc subunit and the second Fc subunit include complementary dimerization selectivity modules that promote dimerization between the first Fc subunit and the second Fc subunit.

In some embodiments, the first Fc subunit and the second Fc subunit each includes a protuberance or a cavity, and in which: (a) the first Fc subunit includes a protuberance, and the second Fc subunit includes a cavity; or (a) the first Fc subunit includes a cavity, and the second Fc subunit includes a protuberance. In some embodiments, the first Fc subunit includes a protuberance, and the second Fc subunit includes a cavity.

In some embodiments, the first Fc subunit and the second Fc subunit include amino acid substitutions: (a) 354C and 366W in the first Fc subunit; and 349C, 366S, 368A, and 407V in the second Fc subunit; (b) 349° C., 366S, 368A, and 407V in the first Fc subunit; and 354C and 366W in the second Fc subunit; (c) 366W in the first Fc subunit; and 366S, 368A, and 407V in the second Fc subunit; (d) 366S, 368A, and 407V in the first Fc subunit; and 366W in the second Fc subunit; (e) 364H and 405A in the first Fc subunit; and 349T and 394F in the second Fc subunit; (f) 349T and 394F in the first Fc subunit; and 364H and 405A in the second Fc subunit; (g) 405L in the first Fc subunit; and 409R in the second Fc subunit; (h) 409R in the first Fc subunit; and 405L in the second Fc subunit; (i) 366L, 392L, and 394W in the first Fc subunit; and 351Y, 405A, and 407V in the second Fc subunit; (j) 351Y, 405A, and 407V in the first Fc subunit; and 366L, 392L, and 394W in the second Fc subunit; (k) 360E and 409W in the first Fc subunit; and 347R, 399V, and 405T in the second Fc subunit; (l) 347R, 399V, and 405T in the first Fc subunit; and 360E and 409W in the second Fc subunit; (m) 349C, 360E, and 409W in the first Fc subunit; and 347R, 354C, 399V, and 405T in the second Fc subunit; (n) 347R, 354C, 399V, and 405T in the first Fc subunit; and 349C, 360E, and 409W in the second Fc subunit; (o) 370E and 409W in the first Fc subunit; and 357N, 399V, and 405T in the second Fc subunit; (p) 357N, 399V, and 405T in the first Fc subunit; and 370E and 409W in the second Fc subunit; (q) 360D, 399M, and 407A in the first Fc subunit; and 345R, 347R, 366V, and 409V in the second Fc subunit; (r) 345R, 347R, 366V, and 409V in the first Fc subunit; and 360D, 399M, and 407A in the second Fc subunit; (s) 349S, 366M, 370Y, and 409V in the first Fc subunit; and 356G, 357D, 364Q, and 407A in the second Fc subunit; (t) 356G, 357D, 364Q, and 407A in the first Fc subunit; and 349S, 366M, 370Y, and 409V in the second Fc subunit; (u) 368D and 370S in the first Fc subunit; and 356Q and 364K in the second Fc subunit; (v) 356Q and 364K in the first Fc subunit; and 368D and 370S in the second Fc subunit; (w) 366Y in the first Fc subunit; and 366S, 368A, and 407T in the second Fc subunit; or (x) 366S, 368A, and 407T in the first Fc subunit; and 366Y in the second Fc subunit, and in which the amino acid positions are based on EU numbering. In some embodiments, the first Fc subunit includes amino acid substitutions 354C and 366W, and the second Fc subunit includes amino acid substitutions 349C, 366S, 368A, and 407V, in which the amino acid positions are based on EU numbering.

In some embodiments, the first Fc subunit includes a first charged region, and the second Fc subunit includes a second charged region, and in which the first charged region forms a charge pair with the second charged region. In some embodiments, the first charged region includes a basic amino acid residue, and the second charged region includes an acidic amino acid residue. In some embodiments, the first charged region includes an acidic amino acid residue, and the second charged region includes a basic amino acid residue.

In some embodiments, the first Fc subunit and the second Fc subunit include amino acid substitutions: (a) K409D in the first Fc subunit; and D399K in the second Fc subunit; (b) D399K in the first Fc subunit; and K409D in the second Fc subunit; (c) K390D and K409D in the first Fc subunit; and E356K and D399K in the second Fc subunit; (d) E356K and D399K in the first Fc subunit; and K390D and K409D in the second Fc subunit; (e) K390D and K409D in the first Fc subunit; and E357K and D399K in the second Fc subunit; (f) E357K and D399K in the first Fc subunit; and K390D and K409D in the second Fc subunit; (g) K370D and K409D in the first Fc subunit; and E357K and D399K in the second Fc subunit; (h) E357K and D399K in the first Fc subunit; and K370D and K409D in the second Fc subunit; (i) R392D and K409D in the first Fc subunit; and E356K and D399K in the second Fc subunit; (j) E356K and D399K in the first Fc subunit; and R392D and K409D in the second Fc subunit; (k) L351 D and L368E in the first Fc subunit; and L351K and T366K in the second Fc subunit; or (l) L351K and T366K in the first Fc subunit; and L351 D and L368E in the second Fc subunit, in which the amino acid positions are based on EU numbering.

In some embodiments, the first Fc subunit or the second Fc subunit includes the amino acid substitutions 435R and 436F, in which the amino acid positions are based on EU numbering. In some embodiments, the second Fc subunit includes the amino acid substitutions 435R and 436F.

In some embodiments, the first Fc subunit and the second Fc subunit include at least one of (e.g., one, two, three, four, five, six, seven, or all eight of) the following amino acid substitutions: (a) 252Y and, optionally, at least one amino acid substitution selected from the group consisting of 251 D or 251 E; 285N or 285D; 286D, 286Y, 286F, 286L, or 286W; 307Q; 308P; 315D; 426L, 426H, 426F, or 426Y; 430A or 430K; 433K; 435Y; and 436H; (b) 252M and, optionally, at least one amino acid substitution selected from the group consisting of 251 D or 251 E; 256D or 256F; 285N or 285D; 286D, 286Y, 286F, 286L, or 286W; 307Q; 308P; 315D; 426L, 426H, 426F, or 426Y; 430A or 430K; 433K; 435Y; and 436H; (c) 434H and, optionally, at least one amino acid substitution selected from the group consisting of 286Y, 286F, 286L, or 286W; and 426Y, 426F, 426L, or 426W; (d) 434R; (e) 426Y and, optionally, at least one amino acid substitution selected from the group consisting of 286F, 286W, 286L, or 286Y; 312P; 434R; and 436H; (f) 426H and, optionally, at least one amino acid substitution selected from the group consisting of 286F, 286W, 286L, or 286Y; 312P; 434R; and 436H; (g) 426F and, optionally, at least one amino acid substitution selected from the group consisting of 286F, 286W, 286L, or 286Y; 312P; 434R; and 426H; or (h) 434R and, optionally, at least one amino acid substitution selected from the group consisting of 286L; 286Y; 312P; and 436H, in which the amino acid positions are based on EU numbering. In some embodiments, the first Fc subunit and the second Fc subunit include the amino acid substitutions 426Y and 286L.

In some embodiments, the first Fc subunit and the second Fc subunit include the amino acid substitutions 234A and 235A, in which the amino acid positions are based on EU numbering.

In some embodiments, the first Fc subunit includes the amino acid substitutions 234A, 235A, 286L, 354C, 366W, and 426Y; and the second Fc subunit includes the amino acid substitutions 234A, 235A, 286L, 349C, 366S, 368A, 407V, 426Y, 435R, and 436F, in which the amino acid positions are based on EU numbering.

In some embodiments, the first Fc subunit and the second Fc subunit are variants of wild type canine IgGA, IgGB, IgGC, or IgGD Fc.

In some embodiments, the first Fc subunit and the second Fc subunit include a sequence having at least 80% identity (e.g., 85%, 90%, 95%, 97%, 99%, or 100% identity) to any one of SEQ ID NOs: 41-44.

In some embodiments, the first CH1 is linked to the first Fc subunit by a first hinge region, and the second CL is linked to the second Fc subunit by a second hinge region. In some embodiments, the first CH1 is linked at the C-terminus to the N-terminus of the first hinge region, and the first hinge region is linked at the C-terminus to the N-terminus of the first Fc subunit. In some embodiments, the second CL is linked at the C-terminus to the N-terminus of the second hinge region, and the second hinge region is linked at the C-terminus to the N-terminus of the second Fc subunit.

In some embodiments, the first hinge region and the second hinge region include a sequence of: (a) VPKRENGRVPRPPDCPKCP (SEQ ID NO: 35); (b) VFNECRCTDTPPCP (SEQ ID NO: 34); (c) PKRENGRVPRPPDCPKCP (SEQ ID NO: 45); (d) FNECRCTDTPPCPVPEP (SEQ ID NO: 46); (e) PKRENGRVPRPPDCPKCPAPEM (SEQ ID NO: 47); (f) AKECECKCNCNNCPCPGCGL (SEQ ID NO: 48); (g) PKESTCKCISPCPVPES (SEQ ID NO: 49); (h) PKESTCKCIPPCPVPES (SEQ ID NO: 50); (i) KTDHPPGPKPCDCPKCP (SEQ ID NO: 51); or (j) KTASTIESKTGEGPKCP (SEQ ID NO: 52). In some embodiments, the first hinge region and the second hinge region include the sequence of VPKRENGRVPRPPDCPKCP (SEQ ID NO: 35).

In some embodiments, the first light chain includes a sequence having at least 80% identity (e.g., at least 85%, 90%, 95%, 97%, 99%, or 100% identity) to SEQ ID NO: 28 or 29. In some embodiments, the first light chain includes the sequence of SEQ ID NO: 28 or 29. In some embodiments, the first light chain includes the sequence of SEQ ID NO: 28.

In some embodiments, the first heavy chain includes a sequence having at least 80% identity (e.g., at least 85%, 90%, 95%, 97%, 99%, or 100% identity) to SEQ ID NO: 30. In some embodiments, the first heavy chain includes the sequence of SEQ ID NO: 30.

In some embodiments, the second light chain includes a sequence having at least 80% identity (e.g., at least 85%, 90%, 95%, 97%, 99%, or 100% identity) to any one of SEQ ID NOs: 1-3 and 12-15. In some embodiments, the second light chain includes a sequence of any one of SEQ ID NOs: 1-3 and 12-15. In some embodiments, the second light chain includes the sequence of SEQ ID NO: 14 or 15.

In some embodiments, the second heavy chain includes a sequence having at least 80% identity (e.g., at least 85%, 90%, 95%, 97%, 99%, or 100% identity) to any one of SEQ ID NOs: 4-11. In some embodiments, the second heavy chain includes a sequence of any one of SEQ ID NOs: 4-11. In some embodiments, the second heavy chain includes the sequence of SEQ ID NO: 5, 8, or 10. In some embodiments, the second heavy chain includes the sequence of SEQ ID NO: 10.

In some embodiments, the first light chain includes a sequence having at least 80% identity (e.g., at least 85%, 90%, 95%, 97%, 99%, or 100% identity) to SEQ ID NO: 28 or 29; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to any one of SEQ ID NOs: 1-3 and 12-15; and the second heavy chain includes a sequence having at least 80% identity (e.g., 85%, 90%, 95%, 97%, 99%, or 100% identity) to any one of SEQ ID NOs: 4-11.

In some embodiments, the first light chain includes the sequence of SEQ ID NO: 28 or 29; the first heavy chain includes the sequence of SEQ ID NO: 30; the second light chain includes a sequence of any one of SEQ ID NOs: 1-3 and 12-15; and the second heavy chain includes a sequence of any one of SEQ ID NOs: 4-11.

In some embodiments, the first light chain includes the sequence of SEQ ID NO: 28; the first heavy chain includes the sequence of SEQ ID NO: 30; the second light chain includes the sequence of SEQ ID NO: 14 or 15; and the second heavy chain includes the sequence of SEQ ID NO: 5, 8, or 10.

In some embodiments, the first antigen and the second antigen are each independently selected from the group consisting of nerve growth factor (NGF), Activin receptor type IIA (RIIA), Activin receptor type IIB (RIIB), TrKA, ADAMTS, IL-1, IL-2, IL-4, IL-4R, angiotensin type 1 (AT1) receptor, angiotensin type 2 (AT2) receptor, IL-5, IL-12, IL-13, IL-31, IL-31R, IL-33, CD3, CD20, CD47, CD52, and a complement system component. In some embodiments, the complement system component is selected from the group consisting of a complement C1 complex component (e.g., C1q, C1 r, and C1s), C2, C3, C4, C5, C6, C7, C8, C9, Factor H, Factor I, CD55, CD59, Factor B, Factor D, properdin, mannose-binding lectin, ficolin, and mannan-binding lectin serine protease (MASP) (e.g., MASP-1, MASP-2, and MASP-3).

In some embodiments, the first VL and the second VL each includes a set of complementarity-determining region-light chain 1 (CDR-L1), CDR-L2, and CDR-L3 sequences independently selected from Table 2 below, and the first VH and the second VH each includes a set of complementarity-determining region-heavy chain 1 (CDR-H1), CDR-H2, and CDR-H3 sequences independently selected from Table 2 below.

In a second aspect, the disclosure features an antibody or antigen-binding fragment thereof capable of specifically binding to nerve growth factor (NGF), in which the antibody or antigen-binding fragment thereof includes a complementarity-determining region-heavy chain 1 (CDR-H1), CDR-H2, and CDR-H3, and a complementarity-determining region-heavy chain 1 (CDR-L1), CDR-L2, and CDR-L3, in which: (a) the CDR-H1 includes the amino acid sequence of SEQ ID NO: 55 or a variant thereof with up to two (e.g., one or two) conservative amino acid substitutions; (b) the CDR-H2 includes the amino acid sequence of SEQ ID NO: 56 or a variant thereof with up to two (e.g., one or two) conservative amino acid substitutions; (c) the CDR-H3 includes the amino acid sequence of SEQ ID NO: 57 or a variant thereof with up to two (e.g., one or two) conservative amino acid substitutions; (d) the CDR-L1 includes the amino acid sequence of SEQ ID NO: 58 or a variant thereof with up to two (e.g., one or two) conservative amino acid substitutions; (e) the CDR-L2 includes the amino acid sequence ATS or a variant thereof with up to two (e.g., one or two) conservative amino acid substitutions; and (f) the CDR-L3 includes the amino acid sequence of SEQ ID NO: 59 or a variant thereof with up to two (e.g., one or two) conservative amino acid substitutions.

In some embodiments, (a) the CDR-H1 includes the amino acid sequence of SEQ ID NO: 55; (b) the CDR-H2 includes the amino acid sequence of SEQ ID NO: 56; (c) the CDR-H3 includes the amino acid sequence of SEQ ID NO: 57; (d) the CDR-L1 includes the amino acid sequence of SEQ ID NO: 58; (e) the CDR-L2 includes the amino acid sequence ATS; and (f) the CDR-L3 includes the amino acid sequence of SEQ ID NO: 59.

In some embodiments, the antibody or antigen-binding fragment thereof includes a heavy chain variable region (VH) containing a sequence having at least 80% identity (e.g., 85%, 90%, 95%, 97%, 99%, or 100% identity) to SEQ ID NO: 60, and a light chain variable region (VL) containing a sequence having at least 80% identity (e.g., 85%, 90%, 95%, 97%, 99%, or 100% identity) to SEQ ID NO: 61. In some embodiments, the VH includes the sequence of SEQ ID NO: 60 and the VL includes the sequence of SEQ ID NO: 61.

In some embodiments, the antibody or antigen-binding fragment thereof is a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, an antibody-like protein scaffold, a Fv fragment, a Fab fragment, a F(ab′)2 molecule, or a tandem scFv (taFv).

In some embodiments, the antibody or antigen-binding fragment thereof is of IgG, IgA, IgM, IgD, or IgE isotype. In some embodiments, the antibody or antigen-binding fragment thereof is of IgG isotype. In some embodiments, the antibody or antigen-binding fragment thereof is of canine IgGA, IgGB, IgGC, or IgGD subclass. In some embodiments, the antibody or antigen-binding fragment thereof is of canine IgGB subclass.

In some embodiments, the antibody or antigen-binding fragment thereof includes a canine Fc domain variant containing at least one of (e.g., one, two, three, four, five, six, seven, or all eight of) the following amino acid substitutions: (a) 252Y and, optionally, at least one amino acid substitution selected from the group consisting of 251 D or 251 E; 285N or 285D; 286D, 286Y, 286F, 286L, or 286W; 307Q; 308P; 315D; 426L, 426H, 426F, or 426Y; 430A or 430K; 433K; 435Y; and 436H; (b) 252M and, optionally, at least one amino acid substitution selected from the group consisting of 251 D or 251 E; 256D or 256F; 285N or 285D; 286D, 286Y, 286F, 286L, or 286W; 307Q; 308P; 315D; 426L, 426H, 426F, or 426Y; 430A or 430K; 433K; 435Y; and 436H; (c) 434H and, optionally, at least one amino acid substitution selected from the group consisting of 286Y, 286F, 286L, or 286W; and 426Y, 426F, 426L, or 426W; (d) 434R; (e) 426Y and, optionally, at least one amino acid substitution selected from the group consisting of 286F, 286W, 286L, or 286Y; 312P; 434R; and 436H; (f) 426H and, optionally, at least one amino acid substitution selected from the group consisting of 286F, 286W, 286L, or 286Y; 312P; 434R; and 436H; (g) 426F and, optionally, at least one amino acid substitution selected from the group consisting of 286F, 286W, 286L, or 286Y; 312P; 434R; and 426H; or (h) 434R and, optionally, at least one amino acid substitution selected from the group consisting of 286L; 286Y; 312P; and 436H, in which the amino acid positions are based on EU numbering. In some embodiments, the canine Fc domain variant includes the amino acid substitutions 426Y and 286L.

In some embodiments, the canine Fc domain variant further includes the amino acid substitutions 234A and 235A, in which the amino acid positions are based on EU numbering.

In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NOs: 41-44. In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NO: 42.

In some embodiments, the antibody or antigen-binding fragment thereof includes a heavy chain containing a sequence having at least 80% identity (e.g., 85%, 90%, 95%, 97%, 99%, or 100% identity) to SEQ ID NO: 26 and a light chain containing a sequence having at least 80% identity (e.g., 85%, 90%, 95%, 97%, 99%, or 100% identity) to SEQ ID NO: 27. In some embodiments, the heavy chain includes the sequence of SEQ ID NO: 26 and the light chain includes the sequence of SEQ ID NO: 27.

In a third aspect, the disclosure features a pharmaceutical composition including the bispecific antibody of the first aspect and a pharmaceutically acceptable excipient.

In a fourth aspect, the disclosure features a pharmaceutical composition including the antibody or antigen-binding fragment thereof of the second aspect, and a pharmaceutically acceptable excipient.

In a fifth aspect, the disclosure features a nucleic acid encoding the bispecific antibody of the first aspect or the antibody or antigen-binding fragment thereof of the second aspect.

In a sixth aspect, the disclosure features a vector including the nucleic acid of the fifth aspect.

In some embodiments, the vector is an expression vector.

In a seventh aspect, the disclosure features an isolated host cell including the nucleic acid of the fifth aspect or the vector of the sixth aspect.

In an eighth aspect, the disclosure features a method of making a bispecific antibody, or an antibody or antigen-binding fragment thereof, the method including: (a) providing the nucleic acid of the fifth aspect or the vector of the sixth aspect; and (b) expressing the nucleic acid or the vector in a host cell culture, thereby producing the bispecific antibody, or the antibody or antigen-binding fragment thereof.

In some embodiments, the method further includes (c) collecting the bispecific antibody or the antibody or antigen-binding fragment thereof produced in (b) from the host cell culture.

In a ninth aspect, the disclosure features a method of treating or preventing a canine disease or disorder in a dog in need thereof, the method including administering an effective amount of the bispecific antibody of the first aspect, the antibody or antigen-binding fragment thereof of the second aspect, or the pharmaceutical composition of the third or fourth aspect.

In some embodiments, the canine disease or disorder is an allergic disease, a chronic pain, an acute pain, a skeletal or musculoskeletal disease, a metabolic disease, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, muscular atrophy, diabetes (e.g., type 1 diabetes or type 2 diabetes), arthritis, anemia, or obesity. A person of ordinary skill in the art understands that the bispecific antibody of the first aspect may be used to treat or prevent a disease, disorder, condition, or symptom in a dog in need of when the bispecific antibody includes at least one (e.g., one or two) antigen-binding moiety that specifically binds to an antigen associated with the disease, disorder, condition, or symptom. For example, a bispecific antibody that includes at least one antigen-binding moiety that specifically binds to NGF may be used to treat or prevent a pain (e.g., a chronic pain, an acute pain, an osteoarthritic pain, a perioperative pain, a dental pain, or a cancer pain) or an inflammatory disease (e.g., rheumatoid arthritis, osteoarthritis, or psoriasis) in a dog in need thereof. A bispecific antibody that includes at least one antigen-binding moiety that specifically binds to Activin RIIA and/or Activin RIIB may be used to treat or prevent a skeletal or musculoskeletal disease (e.g., sarcopenia, cachexia, muscular atrophy, osteoporosis) or a metabolic disease (e.g., obesity, diabetes, or fatty liver disease) in a dog in need thereof. A bispecific antibody that includes at least one antigen-binding moiety that specifically binds to IL-31 or IL-31R may be used to treat or prevent an allergic disease (e.g., atopic dermatitis, asthma, allergic rhinitis), an inflammatory disease (e.g., psoriasis, atopic dermatitis), or an autoimmune disease (e.g., lupus, rheumatoid arthritis) in a dog in need thereof.

In a tenth aspect, the disclosure features a method of treating or preventing a pain in a dog in need thereof, the method including administering an effective amount of the antibody or antigen-binding fragment thereof of the second aspect or the pharmaceutical composition of the fourth aspect to the dog. A person of ordinary skill in the art understands that the antibody or antigen-binding fragment that specifically bind to NGF of the second aspect may be used to treat or prevent a pain (e.g., a chronic pain, an acute pain, an osteoarthritic pain, a perioperative pain, a dental pain, or a cancer pain) or an inflammatory disease (e.g., rheumatoid arthritis, osteoarthritis, or psoriasis) in a dog in need thereof.

In an eleventh aspect, the disclosure features any bispecific antibody of the first aspect, any antibody or antigen-binding fragment thereof of the second aspect, or any pharmaceutical composition of the third or fourth aspect for use in the treatment or prevention of a canine disease or disorder in a dog in need thereof.

In some embodiments, the canine disease or disorder is an allergic disease, a chronic pain, an acute pain, a skeletal or musculoskeletal disease, a metabolic disease, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, muscular atrophy, diabetes (e.g., type 1 diabetes or type 2 diabetes), arthritis, anemia, or obesity.

In a twelfth aspect, the disclosure features any antibody or antigen-binding fragment thereof of the second aspect or the pharmaceutical composition of the fourth aspect for use in the treatment or prevention of a pain in a dog in need thereof.

### BRIEF DESCRIPTION OF THE DRAWINGS

FIG. 1 is an amino acid sequence alignment of canine IGHG2 and human IgG1. “+” indicates similar amino acids. Cysteine residues are bold and underlined. The IgG heavy chain hinge regions predicted by the INTERNATIONAL IMMUNOGENETICS INFORMATION SYSTEM® (IMGT®) are boxed.

FIG. 2 is a schematic representation of CanBiMab. LC=light chain; HC=heavy chain; VL=light chain variable region; VH=heavy chain variable region; CL=light chain constant domain; CH1=heavy chain constant domain 1; CH2=heavy chain constant domain 2; CH3=heavy chain constant domain 3.

FIG. 3 is an amino acid sequence alignment of seven canine VL-CH1 chain constructs with an exemplary human VL-CH1 chain.

FIG. 4 is an amino acid sequence alignment of five canine VH-kappa chain constructs with an exemplary human VH-kappa chain. The IgG heavy chain hinge regions predicted by IMGT® are boxed.

FIG. 5 is an amino acid sequence alignment of three canine VH-lambda chain constructs. The IgG heavy chain hinge regions predicted by IMGT® are boxed.

FIG. 6 is a series of logo plots showing secondary structure features of three exemplary canine VL-CH1 chain constructs based on structural modeling. H=helix; B=isolated beta strand; E=beta strand; G=3-10 helix; I=pi helix; T=turn; S=bend; U=unstructured; X=spacer (for padding sequences).

FIG. 7 is a series of gel images showing sodium dodecyl sulfate-polyacrylamide gel electrophoresis (SDS-PAGE) analysis of ten exemplary CanBiMabs generated using various VLCH1 chains under non-reduced or reduced conditions. Lanes 1-4, 9, and 10 correspond to CanBiMabs generated using VLCH1 chains that ended at P211 residue. Lanes 5-8 correspond to CanBiMabs generated using VLCH1 chains having V212 or V212-K214 residues. The amino acid sequences of the heavy and light chains used to construct the CanBiMab crossed arm in each lane of the SDS-PAGE analysis are: SEQ ID NO: 5 and SEQ ID NO: 12 for Lane 1; SEQ ID NO: 8 and SEQ ID NO: 12 for Lane 2; SEQ ID NO: 5 and SEQ ID NO: 13 for Lane 3; SEQ ID NO: 8 and SEQ ID NO: 13 for Lane 4; SEQ ID NO: 5 and SEQ ID NO: 14 for Lane 5; SEQ ID NO: 8 and SEQ ID NO: 14 for Lane 6; SEQ ID NO: 5 and SEQ ID NO: 15 for Lane 7; SEQ ID NO: 8 and SEQ ID NO: 15 for Lane 8; and SEQ ID NO: 10 and SEQ ID NO: 12 for Lanes 9 and 10. The CanBiMabs corresponding to Lanes 9 and 10 had different variable regions. Ld=ladder.

FIGS. 8A-8D are differential scanning calorimetry (DSC) thermograms showing the thermostability profile of four exemplary CanBiMabs, X_VLCH1_F:X_Vhkappa_Hole_Y:Vk:VH (FIG. 8A), X_VLCH1_F:X_Vhkappa_Hole V:Vk:VH (FIG. 8B), X_VLCH1_G:X_Vhkappa_Hole Y:Vk:VH (FIG. 8C), and X_VLCH1_G:X_Vhkappa_Hole V:Vk:VH (FIG. 8D). Specific thermostability parameters of the CanBiMab constructs are shown in Table 7 below. Cp=molar heat capacity.

FIGS. 9A-9D are chromatographic mass spectra profiles of samples containing four exemplary CanBiMabs, X_VLCH1_F:X_Vhkappa_Hole Y:Vk:VH (FIG. 9A), X_VLCH1_F:X_Vhkappa_Hole V:Vk:VH (FIG. 9B), X_VLCH1_G:X_Vhkappa_Hole Y:Vk:VH (FIG. 9C), and X_VLCH1_G:X_Vhkappa_Hole V:Vk:VH (FIG. 9D).

FIG. 10 is a gel image showing SDS-PAGE analysis of four exemplary CanBiMabs generated using various VLCH1 chains under non-reduced or reduced conditions. Lanes 3-4 and 7-8 correspond to CanBiMabs generated using VLCH1 chains that contain V212-K214 with a lambda variable region and paired with VHlambda_Hole_Y. The amino acid sequences of the heavy and light chains used to construct the CanBiMab crossed arm in each lane of the SDS-PAGE analysis are: SEQ ID NO: 5 and SEQ ID NO: 15 for Lanes 1, 2, 5, and 6; SEQ ID NO: 5 and SEQ ID NO: 10 for Lanes 3, 4, 7, and 8. Ld=ladder.

FIG. 11 is a chromatographic mass spectra profile of a sample containing the exemplary CanBiMab, X_VLCH1_G:X_Vhlambda_Hole Y:Vk:VH. Results of the analysis are summarized in Table 11 below.

FIG. 12 is an amino acid sequence alignment of canine IGHG2 and canine IGHG1. “+” indicates similar amino acids. Cysteine residues are bold and underlined. The IgG heavy chain hinge regions predicted by IMGT® are boxed.

### DETAILED DESCRIPTION OF THE INVENTION

The invention described herein relates to antibodies (e.g., bispecific antibodies) for use in canines, pharmaceutical compositions comprising such antibodies, nucleic acids, vectors, and host cells encoding such antibodies, methods of making the antibodies, and methods of using the antibodies. These antibodies can be used for various preventative, diagnostic, and therapeutic purposes. In some embodiments, the disclosure features bispecific antibodies with a CH1 domain exchanged with a CL domain and/or complementary dimerization selectivity modules. Such modifications promote the correct dimerization between polypeptide chains, thereby increasing the yield and stability of the bispecific antibodies described herein. In some embodiments, the disclosure features antibodies or antigen-binding fragments thereof that specifically bind to NGF. Such antibodies or antigen-binding fragments thereof can be used, e.g., to treat or prevent pain in dogs.

### Definitions

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. All numerical designations, e.g., pH, K_{D}, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about” and that a numerical designation may include numerical values that are rounded to the nearest significant figure. It also is to be understood, although not always explicitly stated, that the reagents described herein are merely exemplary and that equivalents of such are known in the art.

Unless otherwise defined, scientific and technical terms used in connection with the present disclosure shall have the meanings that are commonly understood by those of ordinary skill in the art. Further, unless otherwise required by context or expressly indicated, singular terms shall include pluralities, and plural terms shall include the singular. For any conflict in definitions between various sources or publications, the definition provided herein will control.

It is understood that embodiments of the invention described herein include “comprising,” “consisting,” and “consisting essentially of” aspects and embodiments. As used herein, the singular form “a”, “an,” and “the” includes plural references (e.g., at least one, one or more) unless indicated otherwise. The use of the term “or” herein means “and/or” and is not meant to imply that alternatives are mutually exclusive unless specified otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, “percent (%) sequence identity,” “% identical,” and “homology” with respect to a nucleic acid or polypeptide sequence are defined as the percentage of nucleotides or amino acid residues in a reference sequence that are identical with the nucleotides or amino acid residues in the specific nucleic acid or polypeptide sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for measuring alignment, including any parameters needed to achieve maximal alignment over the full length of sequences being compared. In some embodiments, a variant has at least 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added or deleted at the N- or C-terminus of the polypeptide. In some embodiments, a variant has at least 50% sequence identity, at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 97% sequence identity, at least 98% sequence identity, or at least 99% sequence identity with the sequence of the reference nucleic acid or polypeptide.

As used herein, the term “dimerization selectivity module” refers to a sequence of a Fc subunit that facilitates the favored pairing between two Fc subunits to form an Fc domain or Fc domain variant. “Complementary” dimerization selectivity modules are dimerization selectivity modules that promote or favor the selective interaction of two Fc subunits with each other. Complementary dimerization selectivity modules can have the same or different sequences. Exemplary complementary dimerization selectivity modules are described herein.

As used herein, the term “hole” or “cavity” refers to the substitution of at least one of the original amino acid residues in the CH3 domain of an Fc subunit with a different amino acid residue having a smaller side chain volume than the original amino acid residue, thus creating a three-dimensional hole or cavity in the CH3 domain. The term “original amino acid residue” refers to a naturally occurring amino acid residue encoded by the genetic code of a wild-type CH3 domain.

As used herein, the term “knob” or “protuberance” refers to the substitution of at least one of the original amino acid residues in the CH3 domain of an Fc subunit with a different amino acid residue having a larger side chain volume than the original amino acid residue, thus creating a three-dimensional knob or protuberance in the CH3 domain. The term “original amino acid residues” refers to naturally occurring amino acid residues encoded by the genetic code of a wild-type CH3 domain.

As used herein, the term “knob-in-hole (KiH)” describes an Fc domain or Fc domain variant including two Fc subunits, in which the first Fc subunit includes a “hole” or “cavity” in its CH3 domain, while the second Fc subunit includes a “knob” or “protuberance” in its CH3 domain. In a KiH pair, the knob in the CH3 domain of the first Fc subunit is positioned such that it interacts with the hole of the CH3 domain of the second Fc subunit without significantly perturbing the normal association of the dimer at the inter-CH3 domain interface, thereby promoting heterodimerization of the two Fc subunits.

As used herein, the term “charged region” refers to amino acid substitutions in an Fc subunit within the ring of charged residues at the interface between CH3 domains that promote dimerization of the Fc subunits by forming charge pairs. The term “charge pair” refers to the electrostatic pairing of amino acid residues with opposite charge, e.g., the pairing between a basic amino acid residue in one Fc subunit and an acidic amino acid residue in another Fc subunit.

As used herein, the term “amino acid substitution” refers to the replacement of one amino acid in a polypeptide with another amino acid. In some embodiments, an amino acid substitution is a conservative substitution. Amino acid substitutions may be introduced into a polypeptide screened for a desired activity, for example, retained or improved binding to FcRn, retained or improved antigen binding, decreased immunogenicity, improved antibody-dependent cellular cytotoxicity (ADCC) or (complement-dependent cytotoxicity) CDC, or enhanced pharmacokinetics.

As used herein, the term “conservative substitution” refers to a substitution of one amino acid residue for another amino acid residue that has similar properties such as charge, hydrophobicity, and/or size. For example, amino acids may be grouped according to common side-chain properties: hydrophobic: Norleucine (Nle), Met, Ala, Val, Leu, lie;
neutral hydrophilic: Cys, Ser, Thr, Asn, Gln;
acidic: Asp, Glu;
basic: His, Lys, Arg;
rigid: Gly, Pro;
aromatic: Trp, Tyr, Phe.

Conservative substitutions will entail exchanging a member of one of these classes with another member of the same class. Non-conservative substitutions will entail exchanging a member of one of these classes with another class. In some embodiments, a conservative amino acid substitution refers to a substitution that results in similar properties or functions as another amino acid substitution. For example, a conservative amino acid substitution of A426Y can be A426F, A426T, or A426W. Additional, nonlimiting examples for conservative amino acid substitutions are shown in Table 1 below.

TABLE 1
Examples for conservative amino acid substitutions

| Original residue | Exemplary conservative substitutions |
|---|---|
| Ala (A) | Gly; Val; Leu; Ile; Ser |
| Arg (R) | Lys; His; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Nle |
| Leu (L) | Nle; Ile; Val; Met; Ala; Phe |
| Lys (K) | Arg; His; Gln; Asn |
| Met (M) | Leu; Phe; Ile; Tyr |
| Phe (F) | Trp; Leu; Val; Ile; Ala; Tyr; Met |
| Pro (P) | Ala; Gly |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Nle |

As used herein, the term “affinity” refers to the strength of the sum total of noncovalent interactions between a single binding site of a molecule (e.g., an antibody or a receptor) and its binding partner (e.g., an antigen or a ligand). Unless indicated otherwise, as used herein, “binding affinity” refers to intrinsic binding affinity which reflects a 1:1 interaction between members of a binding pair (e.g., antibody and antigen, receptor and ligand). The affinity of a molecule X for its partner Y can generally be represented by the dissociation constant (K_{D}). Affinity can be measured by common protein-protein interaction tools known in the art, such as, for example, immunoblot, enzyme-linked immunosorbent assay (ELISA), kinetic exclusion assay (KinExA), biolayer interferometry (BLI), or surface plasmon resonance (SPR) devices. Specific illustrative and exemplary embodiments for measuring binding affinity are described in the following.

As used herein, the term “derived from,” with reference to an antibody or a fragment thereof, indicates the original source of the antibody or fragment thereof. For example, an antibody domain (e.g., a CH1 domain, a CL domain, or an Fc domain (including Fc subunits)) derived from a corresponding domain in a canine antibody (e.g., a canine IgG) is one that is originally obtained or isolated from a canine antibody, but may have been further manipulated (e.g., mutated, truncated, elongated, labeled, recombinantly expressed, humanized, etc.).

As used herein, the term “surface plasmon resonance (SPR)” denotes an optical phenomenon that allows for the analysis of real-time biospecific interactions by detection of alterations in protein concentrations within a biosensor matrix, for example, using the BIACORE™ system (BIAcore International AB, a GE Healthcare company, Uppsala, Sweden and Piscataway, N.J.). For further descriptions, see Capelli et al., Trends Anal. Chem. 163: 117079, 2023.

As used herein, the term “amino acid sequence” refers a sequence of amino acids residues in a peptide or protein. The terms “polypeptide” and “protein” are used interchangeably to refer to a polymer of amino acid residues and are not limited to a minimum length. Such polymers of amino acid residues may contain natural or unnatural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to polymerase chain reaction (PCR) amplification.

The term “antibody” herein is used in the broadest sense and refers to various antibody structures, including but not limited to monoclonal antibodies, polyclonal antibodies, multispecific antibodies (e.g., bispecific antibodies), and antibody fragments (e.g., Fab) so long as they exhibit the desired antigen-binding activity.

As used herein, the term “bispecific antibody” refers to an antibody derivative that has, in the same antibody molecule, variable regions that recognize two different epitopes. A bispecific antibody may be an antibody that recognizes two different antigens, or an antibody that recognizes two different epitopes on the same antigen.

As used herein the term “antibody fragment” or “antigen-binding fragment” refers to a molecule other than a full-length antibody that comprises a portion of a full-length antibody that binds the antigen to which the full-length antibody binds. In some embodiments, antibody fragments include but are not limited to Fab; single chain variable fragment (e.g., scFv); Fv; Fab′; Fab′-SH; F(ab′)₂; nanobody; diabody; and multispecific antibodies formed from antibody fragments.

The terms “full-length antibody” and “whole antibody” are used herein interchangeably to refer to an antibody having a structure substantially similar to a native antibody structure or having heavy chains that contain an Fc domain or Fc domain variant as defined herein.

The terms “nanobody,” “VHH,” “VHH domain,” “VHH antibody fragment,” and “single domain antibody” as interchangeably used herein denote the variable region of the single heavy chain of antibodies of the type of those found in Camelidae, which are typically found in natural form to lack light chains. Suitable nanobodies will be familiar to persons skilled in the art, illustrated examples of which include nanobodies of camels, dromedaries, llamas, and alpacas. However, the single domain antibody may be from non-Camelidae sources as well.

As used herein, the term “antigen-binding moiety” refers to a part of a compound or a molecule that specifically binds to a target epitope, antigen, ligand, or receptor. Antigen-binding moiety include, but are not limited to, antibodies (e.g., monoclonal, polyclonal, recombinant, and chimeric antibodies), antibody fragments or portions thereof (e.g., Fab, scFv, Fv, Fab′, Fab′-SH, F(ab′)₂, nanobody, and diabody), receptors or fragments thereof (e.g., an extracellular domain of a canine receptor protein), ligands, aptamers, and other molecules having an identified binding partner.

As used herein, the term “chimeric” antibody refers to an antibody in which a portion of the heavy and/or light chain is derived from a particular source or species, while the remainder of the heavy and/or light chain is derived from a different source or species.

As used herein, the term “Fc domain” or “Fc polypeptide” refers to a C-terminal region of an immunoglobulin heavy chain that contains at least a portion of the constant region. The term “Fc domain variant” refers to a variant of a Fc domain (e.g., a variant of a canine Fc domain) having a substitution or substitutions relative to the wild type Fc domain (e.g., the wild type canine Fc domain). The terms “wild type canine Fc domain” refers to the native Fc domain of a canine antibody (e.g., a canine IgGA, IgGB, IgGC, or IgGD antibody). The term “canine Fc domain variant” refers to a variant of the Fc domain of a canine antibody having a substitution or substitutions relative to the wild type canine Fc domain. In some embodiments, the canine Fc domain sequences are from a canine (e.g., dog) IgG (e.g., IgGA, IgGB, IgGC, or IgGD). In some embodiments, an Fc domain comprises a CH2 domain and a CH3 domain, but does not comprise a CH1 domain, a hinge region, or a CL domain. In some embodiments, an Fc domain comprises a hinge region, a CH2 domain, and a CH3 domain, but does not comprise a CH1 domain or a CL domain. In some embodiments, an Fc domain comprises a CH1 domain, a hinge region, a CH2 domain, and a CH3 domain, with or without a CL domain. In some embodiments, an Fc domain comprises a CH1 domain, a hinge region, a CH2 domain, a CH3 domain, and a CL domain. For example, the CL domain may be linked to the CH1 domain via a disulfide bridge. In some embodiments, an Fc domain, such as an IgG Fc domain, lacks one or more C-terminal amino acids, such as 1 to 20, 1 to 15, 1 to 10, 1 to 5, or 1 to 2 amino acids, while retaining biological activity. In some embodiments, the biological activity of an Fc domain is the ability to bind FcRn. Unless otherwise specified herein, numbering of amino acid residues in the Fc domain is according to the EU numbering system, also called the EU index, as described in Kabat et al. Sequences of Proteins of Immunological Interest, 5th Ed. Public Health Service, National Institutes of Health, Bethesda, MD, 1991.

As used herein, the term “Fc subunit” refers to a polypeptide chain that includes at least a CH2 domain and a CH3 domain or functional fragments thereof (e.g., fragments that that capable of (i) dimerizing with another Fc subunit to form an Fc domain and (ii) binding to an Fc receptor). In some embodiments, a n Fc subunit further includes a hinge region. In some embodiments, the Fc subunit is derived from immunoglobulin. The Fc subunit can be any immunoglobulin antibody isotype, including, e.g., IgG. Additionally, the Fc subunit can be an IgG subclass, e.g., IgGA, IgGB, IgGC, or IgGD in dogs. In some embodiments, an Fc subunit does not include any portion of an immunoglobulin that is capable of antigen binding, e.g., a variable region or a complementarity-determining region (CDR). In some embodiments, an Fc subunit includes a portion of an immunoglobulin that can act as an antigen-recognition region, e.g., a variable region or a CDR. In some embodiments, an Fc subunit includes a single-domain antibody, e.g., a VHH domain.

As used herein, the term “wild type” refers to a non-mutated version of a polypeptide that occurs in nature, or a fragment thereof. A wild type polypeptide may be produced recombinantly. In some embodiments, a wild type canine (e.g., dog) IgG Fc domain comprises the amino acid sequence of any one of SEQ ID NOs: 41-44.

As used herein, the term “disease” or “disorder” refers to any condition that would benefit from treatment including, but not limited to, chronic and acute disorders or diseases including those pathological conditions which predispose the mammal to the disorder in question.

As used herein, the term “cancer” refers to or describes the physiological condition in mammals that is typically characterized by unregulated cell growth/proliferation. Examples of cancer include, but are not limited to, myeloma, carcinoma, lymphoma (e.g., Hodgkin's and non-Hodgkin's lymphoma), blastoma, sarcoma (e.g., hemangiosarcoma, osteosarcoma, soft-tissue sarcoma, and histiocytic sarcoma), leukemia, head and neck squamous cell carcinoma, salivary adenocarcinoma, breast cancer, mastocytoma, melanoma, lung cancer (e.g., small-cell lung cancer, non-small cell lung cancer, adenocarcinoma of the lung, and squamous carcinoma of the lung), cancer of the peritoneum, hepatocellular carcinoma, squamous cell carcinoma, meningioma, glioma, gastric cancer, intestinal cancer, colon cancer, colorectal cancer, pancreatic adenocarcinoma, glioblastoma, cervical cancer, endometrial or uterine carcinoma, ovarian cancer, bladder cancer, prostatic carcinoma, kidney or renal cancer, vulval cancer, thyroid cancer, and transitional cell carcinoma.

As used herein, the term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

As used herein, the term “effector functions” refer to those biological activities attributable to the Fc domain of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, an “effective amount” of a composition, for example, an antibody (e.g., a bispecific antibody, an antibody, or an antigen-binding fragment thereof) of the present disclosure or a composition (e.g., pharmaceutical composition) thereof, refers to at least the minimum amount required to achieve the desired therapeutic or prophylactic result, such as a measurable improvement or prevention of a particular disorder (e.g., any disorder affecting a canine, e.g., a cell proliferative disorder, e.g., cancer). An effective amount herein may vary according to factors such as the disease state, age, sex, and weight of the animal, and the ability of the polypeptide (e.g., a bispecific antibody, an antibody, or an antigen-binding fragment thereof) to elicit a desired response in the animal. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The terms “host cell” and “host cell culture” are herein used interchangeably and refer to cells into which exogenous nucleic acid has been introduced, including the progeny of such cells. Host cells include bacterial (e.g., E. coli cells) and eukaryotic cells. In some embodiments, host cells include yeast cells (e.g., Pichia (see, e.g., Powers et al., 2001, J Immunol Methods. 251: 123-135), Hanseula, or Saccharomyces). In some embodiments, host cells also include “transformants” and “transformed cells,” which include the primary transformed cell lines (e.g., CHO, 293E, COS, 293T, and HeLa) and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

As used herein, the term “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, the term “pharmaceutical composition” refers to a preparation which is in such form as to permit the biological activity of an active ingredient contained therein to be effective, and which contains no additional components which are unacceptably toxic to a subject to which the formulation would be administered.

As used herein, the term “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the bispecific binding agents of the invention are used to delay development of a disease or to slow the progression of a disease.

As used herein, the term “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the canine does not develop the disease. For example, a late-stage cancer, such as development of metastasis, may be delayed.

As used herein, the term “epitope” refers to the particular site or sites on an antigen molecule to which an antibody (e.g., an antibody or antigen-binding fragment thereof) or other binding agent binds. For example, an epitope may be a linear epitope or a conformational epitope.

As used herein, the terms “reduce” and “inhibit” refer to the ability to cause an overall decrease, for example, of 20% or greater, of 50% or greater, or of 75%, 85%, 90%, 95%, or greater, e.g., as compared to a reference or control.

As used herein, the terms “increase” and “enhance” refer to the ability to cause an overall increase, for example, of 20% or greater, of 50% or greater, or of 75%, 85%, 90%, 95%, or greater, e.g., as compared to a reference or control.

As used herein, the terms “variable region” and “variable region” refer to the domain of an antibody heavy or light chain that is involved in binding the antibody to antigen. The variable regions of the heavy chain and light chain (VH and VL, respectively) of a native antibody generally have similar structures, with each domain comprising four conserved framework regions (FRs) and three hypervariable regions (HVRs). See, e.g., Kindt et al., 2007, Kuby Immunology, 6th ed. W.H. Freeman and Co., page 91. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., 1993, J. Immunol. 150: 880-887; and Clarkson et al., 1991, Nature 352: 624-628.

A “variant” of a polypeptide (referred to as the reference polypeptide) is a polypeptide that differs from the reference polypeptide by single or multiple non-native amino acid substitutions, deletions, and/or additions. In some embodiments, a variant of a reference polypeptide retains at least one biological activity of the reference polypeptide. In some embodiments, a variant of a polypeptide has a biological activity that the reference polypeptide substantially lacks. For example, a “canine Fc domain variant” comprises an amino acid sequence which differs from that of a wild type canine Fc domain by at least one amino acid modification, preferably one or more amino acid substitution(s). Preferably, the canine Fc domain variant has at least one amino acid substitution compared to a wild type canine Fc domain, e.g., from one to ten amino acid substitutions, and preferably from one to five amino acid substitutions in a wild type canine Fc domain. The canine Fc domain variant herein will preferably possess at least 80% homology with a wild type canine Fc domain, and most preferably at least 90% homology therewith, more preferably at least 95% homology therewith. In some embodiments, the canine Fc domain is a canine IgGA Fc domain variant, a canine IgGB Fc domain variant, a canine IgGC Fc domain variant, or a canine IgGD Fc domain variant. In some embodiments, the Fc domain variant (e.g., a canine Fc domain variant) comprises the hinge, CH2, and CH3, but does not comprise CH1 or CL. In some embodiments, the Fc domain variant (e.g., a canine Fc domain variant) comprises CH2 and CH3, but does not comprise CH1, the hinge, or CL. In some embodiments, the Fc domain variant (e.g., a canine Fc domain variant) comprises CH1, hinge, CH2, and CH3, with or without CL. In some embodiments, the Fc domain variant (e.g., a canine Fc domain variant) comprises CH1, hinge, CH2, CH3, and CL. For example, CL may be linked to CH1 via a disulfide bridge.

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors can direct the expression of nucleic acids to which they are operatively linked. Such vectors are referred to herein as “expression vectors.”

As used herein, the term “administering” refers to a method of giving a dosage of a compound (e.g., a bispecific binding agent of the present disclosure, e.g., a bispecific antibody, an antibody, or an antigen-binding fragment thereof) or a composition (e.g., a pharmaceutical composition, e.g., a pharmaceutical composition including a bispecific antibody, an antibody, or an antigen-binding fragment thereof of the present disclosure) to a subject. The compositions utilized in the methods described herein can be administered, for example, parenterally, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The administration may be local or systemic. The method of administration can vary depending on various factors (e.g., the compound or composition being administered and the severity of the condition, disease, or disorder being treated).

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

### Structural Features of Canine Antibodies

Canine IgG antibodies include four types of heavy chains referred to as A, B, C, and D. These heavy chains represent four different subclasses of canine IgG, which are referred to as IgGA, IgGB, IgGC and IgGD. The amino acid and DNA sequences for these heavy chains are available from Tang et al., Vet. Immunol. Immunopathol. 80: 259-270, 2001, and the GENBANK® database. For example, the amino acid sequence of IgGA heavy chain has accession number AAL35301.1, IgGB has accession number AAL35302.1, IgGC has accession number AAL35303.1, and IgGD has accession number AAL35304.1. The heavy chain of canine IgG includes a CH1 domain and a Fc domain connected by a hinge region. The Fc domain further includes a CH2 domain and a CH3 domain.

Canine antibodies also include two types of light chains: kappa and lambda. The DNA and amino® acid sequence of these light chains can also be obtained from GENBANK® database. For example, the dog kappa light chain amino acid sequence has accession number ABY57289.1 and the dog lambda light chain has accession number ABY55569.1. The light chain of canine IgG includes a CL domain.

### Canine IgG CL Domain

The amino acid sequence of the CL domain of wild type canine kappa light chain is provided below:

(SEQ ID NO: 28)
RNDAQPAVYLFQPSPDQLHTGSASVVCLLNSFYPKDINVKWKVDGVIQ
DTGIQESVTEQDKDSTYSLSSTLTMSSTEYLSHELYSCEITHKSLPST
LIKSFORSECQRVD

The amino acid sequence of the CL domain of wild type canine lambda light chain is provided below:

(SEQ ID NO: 29)
GQPKASPSVTLFPPSSEELGANKATLVCLISDFYPSGVTVAWKADGSP
VTQGVETTKPSKQSNNKYAASSYLSLTPDKWKSHSSFSCLVTHEGSTV
EKKVAPAECS

It is to be understood that the CL domains may include one to six (e.g., 1, 2, 3, 4, 5, or 6) additional amino acids or deletions at their N and/or C-terminus.

### Canine IgG CH1 Domain

The amino acid sequence of the CH1 domain of wild type canine IgGA is provided below:

(SEQ ID NO: 70)
ASTTAPSVFPLAPSCGSTSGSTVALACLVSGYFPEPVTVSWNSGSLTS
GVHTFPSVLQSSGLHSLSSMVTVPSSRWPSETFTCNVVHPASNTKVDK
P

The amino acid sequence of the CH1 domain of wild type canine IgGB is provided below:

(SEQ ID NO: 71)
ASTTAPSVFPLAPSCGSTSGSTVALACLVSGYFPEPVTVSWNSGSLTS
GVHTFPSVLQSSGLYSLSSMVTVPSSRWPSETFTCNVAHPASKTKVDK
P

The amino acid sequence of the CH1 domain of wild type canine IgGC is provided below:

(SEQ ID NO: 72)
ASTTAPSVFPLAPSCGSQSGSTVALACLVSGYIPEPVTVSWNSVSLTS
GVHTFPSVLQSSGLYSLSSMVTVPSSRWPSETFTCNVAHPATNTKVDK
P

The amino acid sequence of the CH1 domain of wild type canine IgGD is provided below:

(SEQ ID NO: 73)
ASTTAPSVFPLAPSCGSTSGSTVALACLVSGYFPEPVTVSWNSGSLTS
GVHTFPSVLQSSGLYSLSSTVTVPSSRWPSETFTCNVVHPASNTKVDK
P

It is to be understood that the CH1 domains may include one to six (e.g., 1, 2, 3, 4, 5, or 6) additional amino acids or deletions at their N and/or C-terminus.

The last 15 amino acid residues at the C-terminal of the CH1 domain of canine IgGB are referred to as C197, N198, V199, A200, H201, P202, A203, S204, K205, T206, K207, V208, D209, K210, and P211, respectively. It can be noted above that all canine CH1 sequences have a cysteine at position 197, a valine at position 208, an aspartic acid at position 209, a lysine at position 210, and a proline at position 211.

### Canine IgG Heavy Chain Hinge Region

The amino acid sequence of the hinge region of wild type canine IgGA is provided below:

(SEQ ID NO: 34)
VFNECRCTDTPPCP

The amino acid sequence of the hinge region of wild type canine IgGB is provided below:

(SEQ ID NO: 35)
VPKRENGRVPRPPDCPKCP

The amino acid sequence of the hinge region of wild type canine IgGC is provided below:

(SEQ ID NO: 82)
VAKECECKCNCNNCPCP

The amino acid sequence of the hinge region of wild type canine IgGD is provided below:

(SEQ ID NO: 83)
VPKESTCKCISPCP

Unless otherwise specified herein, amino acid residues within the hinge region of canine antibodies are numbered starting at position 212. For example, the first five amino acid residues in the hinge region of canine IgGB are referred to as V212, P213, K214, R215, and E216, respectively.

### Canine IgG Fc Domain

The CH2 domain of a canine antibody heavy chain comprises or consists of amino acids 237 to 340 (according to EU numbering) of the heavy chain. It is to be understood that the CH2 domains may include one to six (e.g., 1, 2, 3, 4, 5, or 6) additional amino acids or deletions at their N and/or C-terminus.

The amino acid sequence of the CH2 domain of wild type canine IgGA is provided below:

(SEQ ID NO: 74)
GPSVLIFPPKPKDILRITRTPEVTCVVLDLGREDPEVQISWFVDGKEVHT
AKTQSREQQFNGTYRVVSVLPIEHQDWLTGKEFKCRVNHIDLPSPIERTI
SKAR

The amino acid sequence of the CH2 domain of wild type canine IgGB is provided below:

(SEQ ID NO: 75)
GPSVFIFPPKPKDTLLIARTPEVTCVVVDLDPEDPEVQISWFVDGKQMQT
AKTQPREEQFNGTYRVVSVLPIGHQDWLKGKQFTCKVNNKALPSPIERTI
SKAR

The amino acid sequence of the CH2 domain of wild type canine IgGC is provided below:

(SEQ ID NO: 76)
GPSVFIFPPKPKDILVTARTPTVTCVVVDLDPENPEVQISWFVDSKQVQT
ANTQPREEQSNGTYRVVSVLPIGHQDWLSGKQFKCKVNNKALPSPIEEII
SKTP

The amino acid sequence of the CH2 domain of wild type canine IgGD is provided below:

(SEQ ID NO: 77)
GPSVFIFPPKPKDILRITRTPEITCVVLDLGREDPEVQISWFVDGKEVHT
AKTQPREQQFNSTYRVVSVLPIEHQDWLTGKEFKCRVNHIGLPSPIERTI
SKAR

The CH3 domain of a canine antibody heavy chain comprises or consists of amino acids 345 to 447 (according to EU numbering) of the heavy chain. It is to be understood that the CH3 domains may include one to six (e.g., 1, 2, 3, 4, 5, or 6) additional amino acids or deletions at their N and/or C-terminus.

The amino acid sequence of the CH3 domain of wild type canine IgGA is provided below:

(SEQ ID NO: 78)
KPSVYVLPPSPKELSSSDTVSITCLIKDFYPPDIDVEWQSNGQQEPERKH
RMTPPQLDEDGSYFLYSKLSVDKSRWQQGDPFTCAVMHETLQNHYTDLSL
SHSPGK

The amino acid sequence of the CH3 domain of wild type canine IgGB is provided below:

(SEQ ID NO: 79)
QPSVYVLPPSREELSKNTVSLTCLIKDFFPPDIDVEWQSNGQQEPESKYR
TTPPQLDEDGSYFLYSKLSVDKSRWQRGDTFICAVMHEALHNHYTQESLS
HSPGK

The amino acid sequence of the CH3 domain of wild type canine IgGC is provided below:

(SEQ ID NO: 80)
QPNVYVLPPSRDEMSKNTVTLTCLVKDFFPPEIDVEWQSNGQQEPESKYR
MTPPQLDEDGSYFLYSKLSVDKSRWQRGDTFICAVMHEALHNHYTQISLS
HSPGK

The amino acid sequence of the CH3 domain of wild type canine IgGD is provided below:

(SEQ ID NO: 81)
QPSVYVLPPSPKELSSSDTVTLTCLIKDFFPPEIDVEWQSNGQPEPESKY
HTTAPQLDEDGSYFLYSKLSVDKSRWQQGDTFTCAVMHEALQNHYTDLSL
SHSPGK

The Fc domain of a canine IgG antibody heavy chain comprises or consists of amino acids 231 to 447 (according to EU numbering) of the heavy chain.

The amino acid sequence of the Fc domain of wild type canine IgGA is provided below:

(SEQ ID NO: 41)
VPEPLGGPSVLIFPPKPKDILRITRTPEVTCVVLDLGREDPEVQISWFVD
GKEVHTAKTQSREQQFNGTYRVVSVLPIEHQDWLTGKEFKCRVNHIDLPS
PIERTISKARGRAHKPSVYVLPPSPKELSSSDTVSITCLIKDFYPPDIDV
EWQSNGQQEPERKHRMTPPQLDEDGSYFLYSKLSVDKSRWQQGDPFTCAV
MHETLQNHYTDLSLSHSPGK

The amino acid sequence of the Fc domain of wild type canine IgGB is provided below:

(SEQ ID NO: 42)
APEMLGGPSVFIFPPKPKDTLLIARTPEVTCVVVDLDPEDPEVQISWFVD
GKQMQTAKTQPREEQFNGTYRVVSVLPIGHQDWLKGKQFTCKVNNKALPS
PIERTISKARGQAHQPSVYVLPPSREELSKNTVSLTCLIKDFFPPDIDVE
WQSNGQQEPESKYRTTPPQLDEDGSYFLYSKLSVDKSRWQRGDTFICAVM
HEALHNHYTQESLSHSPGK

The amino acid sequence of the Fc domain of wild type canine IgGC is provided below:

(SEQ ID NO: 43)
GCGLLGGPSVFIFPPKPKDILVTARTPTVTCVVVDLDPENPEVQISWFVD
SKQVQTANTQPREEQSNGTYRVVSVLPIGHQDWLSGKQFKCKVNNKALPS
PIEEIISKTPGQAHQPNVYVLPPSRDEMSKNTVTLTCLVKDFFPPEIDVE
WQSNGQQEPESKYRMTPPQLDEDGSYFLYSKLSVDKSRWQRGDTFICAVM
HEALHNHYTQISLSHSPGK

The amino acid sequence of the Fc domain of wild type canine IgGD is provided below:

(SEQ ID NO: 44)
VPESLGGPSVFIFPPKPKDILRITRTPEITCVVLDLGREDPEVQI
SWFVDGKEVHTAKTQPREQQFNSTYRVVSVLPIEHQDWLTGKEFK
CRVNHIGLPSPIERTISKARGQAHQPSVYVLPPSPKELSSSDTVT
LTCLIKDFFPPEIDVEWQSNGQPEPESKYHTTAPQLDEDGSYFLY
SKLSVDKSRWQQGDTFTCAVMHEALQNHYTDLSLSHSPGK

### Canine Antibodies

### Bispecific Antibodies

The present disclosure provides a bispecific antibody that includes:
(a) a first antigen-binding moiety that binds to a first antigen, in which the first antigen-binding moiety includes: (i) a first light chain including a first light chain variable region (VL) and a first light chain constant domain (CL); and (ii) a first heavy chain including a first heavy chain variable region (VH), a first heavy chain constant domain 1 (CH1), and a first Fc subunit; and
(b) a second antigen-binding moiety that binds to a second antigen, in which the second antigen-binding moiety includes: (i) a second light chain including a second VL and a second CH1; and (ii) a second heavy chain including a second VH, a second CL, and a second Fc subunit, and
in which the first CH1 and the second CH1 are derived from a CH1 of a canine IgG, the first CL and the second CL are derived from a CL of a canine IgG, and the first Fc subunit and the second Fc subunit are derived from an Fc subunit of a canine IgG.

In some embodiments, the second CH1 includes a sequence having at least 80% identity (e.g., 85%, 90%, 95%, 97%, 99%, or 100% identity) to: (a) a continuous sequence of amino acid residues within SEQ ID NO: 37 that includes at least amino acids 1-83 of SEQ ID NO: 37; (b) a continuous sequence of amino acid residues within SEQ ID NO: 38 that includes at least amino acids 1-83 of SEQ ID NO: 38; (c) a continuous sequence of amino acid residues within SEQ ID NO: 39 that includes at least amino acids 1-X of SEQ ID NO: 39; or (d) a continuous sequence of amino acid residues within SEQ ID NO: 40 that includes at least amino acids 1-X of SEQ ID NO: 40. The sequences of SEQ ID NOs: 37-40 are provided below.

(SEQ ID NO: 37)
ASTTAPSVFPLAPSCGSTSGSTVALACLVSGYFPEPVTVSWNSGS
LTSGVHTFPSVLQSSGLHSLSSMVTVPSSRWPSETFTCNVVHPAS
NTKVDKPVFNECRCTDTPPCPVPEPLGGPSVLIFPPKPKDILRIT
RTPEVTCVVLDLGREDPEVQISWFVDGKEVHTAKTQSREQQFNGT
YRVVSVLPIEHQDWLTGKEFKCRVNHIDLPSPIERTISKARGRAH
KPSVYVLPPSPKELSSSDTVSITCLIKDFYPPDIDVEWQSNGQQE
PERKHRMTPPQLDEDGSYFLYSKLSVDKSRWQQGDPFTCAVMHET
LQNHYTDLSLSHSPGK
(SEQ ID NO: 38)
ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPKCPAPEMLGGPSVFIFPPKPKD
TLLIARTPEVTCVVVDLDPEDPEVQISWFVDGKQMQTAKTQPREE
QFNGTYRVVSVLPIGHQDWLKGKQFTCKVNNKALPSPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPGK
(SEQ ID NO: 39)
ASTTAPSVFPLAPSCGSQSGSTVALACLVSGYIPEPVTVSWNSVS
LTSGVHTFPSVLQSSGLYSLSSMVTVPSSRWPSETFTCNVAHPAT
NTKVDKPVAKECECKCNCNNCPCPGCGLLGGPSVFIFPPKPKDIL
VTARTPTVTCVVVDLDPENPEVQISWFVDSKQVQTANTQPREEQS
NGTYRVVSVLPIGHQDWLSGKQFKCKVNNKALPSPIEEIISKTPG
QAHQPNVYVLPPSRDEMSKNTVTLTCLVKDFFPPEIDVEWQSNGQ
QEPESKYRMTPPQLDEDGSYFLYSKLSVDKSRWQRGDTFICAVMH
EALHNHYTQISLSHSPGK
(SEQ ID NO: 40)
ASTTAPSVFPLAPSCGSTSGSTVALACLVSGYFPEPVTVSWNSGS
LTSGVHTFPSVLQSSGLYSLSSTVTVPSSRWPSETFTCNVVHPAS
NTKVDKPVPKESTCKCISPCPVPESLGGPSVFIFPPKPKDILRIT
RTPEITCVVLDLGREDPEVQISWFVDGKEVHTAKTQPREQQFNST
YRVVSVLPIEHQDWLTGKEFKCRVNHIGLPSPIERTISKARGQAH
QPSVYVLPPSPKELSSSDTVTLTCLIKDFFPPEIDVEWQSNGQPE
PESKYHTTAPQLDEDGSYFLYSKLSVDKSRWQQGDTFTCAVMHEA
LQNHYTDLSLSHSPGK

In some embodiments, the second CH1 includes a sequence containing: (a) a continuous sequence of amino acid residues within SEQ ID NO: 37 that includes at least amino acids 1-83 of SEQ ID NO: 37; (b) a continuous sequence of amino acid residues within SEQ ID NO: 38 that includes at least amino acids 1-83 of SEQ ID NO: 38; (c) a continuous sequence of amino acid residues within SEQ ID NO: 39 that includes at least amino acids 1-X of SEQ ID NO: 39; or (d) a continuous sequence of amino acid residues within SEQ ID NO: 40 that includes at least amino acids 1-X of SEQ ID NO: 40.

In some embodiments, the second CH1 includes a sequence having at least 80% identity (e.g., 85%, 90%, 95%, 97%, 99%, or 100% identity) to: (a) a continuous sequence of amino acid residues within SEQ ID NO: 37 that includes at least amino acids 1-97 of SEQ ID NO: 37; or (b) a continuous sequence of amino acid residues within SEQ ID NO: 38 that includes at least amino acids 1-102 of SEQ ID NO: 38.

In some embodiments, the second CH1 includes a sequence containing: (a) a continuous sequence of amino acid residues within SEQ ID NO: 37 that includes at least amino acids 1-97 of SEQ ID NO: 37; or (b) a continuous sequence of amino acid residues within SEQ ID NO: 38 that includes at least amino acids 1-102 of SEQ ID NO: 38.

In some embodiments, the second CH1 further includes a serine-serine linker at the N-terminus.

In some embodiments, the second CH1 further includes one or more (e.g., one, two, three, four, five, six, seven, eight, nine, ten, eleven, twelve, or more) glycine residues at the C-terminus.

In some embodiments, the second CL includes a sequence having at least 80% identity (e.g., 85%, 90%, 95%, 97%, 99%, or 100% identity) to: (a) a continuous sequence of amino acid residues within SEQ ID NO: 28 that includes at least amino acids 3-106 of SEQ ID NO: 28; or (b) a continuous sequence of amino acid residues within SEQ ID NO: 29 that includes at least amino acids 4-105 of SEQ ID NO: 29.

In some embodiments, the second CL includes a sequence containing: (a) a continuous sequence of amino acid residues within SEQ ID NO: 28 that includes at least amino acids 3-106 of SEQ ID NO: 28; or (b) a continuous sequence of amino acid residues within SEQ ID NO: 29 that includes at least amino acids 4-105 of SEQ ID NO: 29.

In some embodiments, the second CL further includes an alanine-serine linker at the N-terminus.

In some embodiments, the second CL further includes one or more (e.g., one, two, three, four, five, six, seven, eight, nine, ten, eleven, twelve, or more) continuous or discontinuous amino acid residues within the sequence of SEQ ID NO: 31. In some embodiments, the second CL further includes the sequence of SEQ ID NO: 31. The sequences of SEQ ID NO: 31 is provided below.

(SEQ ID NO: 31)
NVAHPASKTKVDKP

In some embodiments, the first Fc subunit and the second Fc subunit include complementary dimerization selectivity modules that promote dimerization between the first Fc subunit and the second Fc subunit.

In some embodiments, the first Fc subunit and the second Fc subunit each includes a protuberance or a cavity, and in which: (a) the first Fc subunit includes a protuberance, and the second Fc subunit includes a cavity; or (a) the first Fc subunit includes a cavity, and the second Fc subunit includes a protuberance. In some embodiments, the first Fc subunit includes a protuberance, and the second Fc subunit includes a cavity.

In some embodiments, the first Fc subunit and the second Fc subunit include amino acid substitutions: (a) 354C and 366W in the first Fc subunit; and 349C, 366S, 368A, and 407V in the second Fc subunit; (b) 349C, 366S, 368A, and 407V in the first Fc subunit; and 354C and 366W in the second Fc subunit; (c) 366W in the first Fc subunit; and 366S, 368A, and 407V in the second Fc subunit; (d) 366S, 368A, and 407V in the first Fc subunit; and 366W in the second Fc subunit; (e) 364H and 405A in the first Fc subunit; and 349T and 394F in the second Fc subunit; (f) 349T and 394F in the first Fc subunit; and 364H and 405A in the second Fc subunit; (g) 405L in the first Fc subunit; and 409R in the second Fc subunit; (h) 409R in the first Fc subunit; and 405L in the second Fc subunit; (i) 366L, 392L, and 394W in the first Fc subunit; and 351Y, 405A, and 407V in the second Fc subunit; (j) 351Y, 405A, and 407V in the first Fc subunit; and 366L, 392L, and 394W in the second Fc subunit; (k) 360E and 409W in the first Fc subunit; and 347R, 399V, and 405T in the second Fc subunit; (l) 347R, 399V, and 405T in the first Fc subunit; and 360E and 409W in the second Fc subunit; (m) 349C, 360E, and 409W in the first Fc subunit; and 347R, 354C, 399V, and 405T in the second Fc subunit; (n) 347R, 354C, 399V, and 405T in the first Fc subunit; and 349C, 360E, and 409W in the second Fc subunit; (o) 370E and 409W in the first Fc subunit; and 357N, 399V, and 405T in the second Fc subunit; (p) 357N, 399V, and 405T in the first Fc subunit; and 370E and 409W in the second Fc subunit; (q) 360D, 399M, and 407A in the first Fc subunit; and 345R, 347R, 366V, and 409V in the second Fc subunit; (r) 345R, 347R, 366V, and 409V in the first Fc subunit; and 360D, 399M, and 407A in the second Fc subunit; (s) 349S, 366M, 370Y, and 409V in the first Fc subunit; and 356G, 357D, 364Q, and 407A in the second Fc subunit; (t) 356G, 357D, 364Q, and 407A in the first Fc subunit; and 349S, 366M, 370Y, and 409V in the second Fc subunit; (u) 368D and 370S in the first Fc subunit; and 356Q and 364K in the second Fc subunit; (v) 356Q and 364K in the first Fc subunit; and 368D and 370S in the second Fc subunit; (w) 366Y in the first Fc subunit; and 366S, 368A, and 407T in the second Fc subunit; or (x) 366S, 368A, and 407T in the first Fc subunit; and 366Y in the second Fc subunit, and in which the amino acid positions are based on EU numbering.

In some embodiments, the first Fc subunit includes amino acid substitutions 354C and 366W, and the second Fc subunit includes amino acid substitutions 349C, 366S, 368A, and 407V.

In some embodiments, the first Fc subunit includes amino acid substitutions 349C, 366S, 368A, and 407V, and the second Fc subunit includes amino acid substitutions 354C and 366W.

In some embodiments, the first Fc subunit includes the amino acid substitution 366W, and the second Fc subunit includes amino acid substitutions 366S, 368A, and 407V.

In some embodiments, the first Fc subunit includes amino acid substitutions 366S, 368A, and 407V, and the second Fc subunit includes the amino acid substitution 366W.

In some embodiments, the first Fc subunit includes amino acid substitutions 364H and 405A, and the second Fc subunit includes amino acid substitutions 349T and 394F.

In some embodiments, the first Fc subunit includes amino acid substitutions 349T and 394F, and the second Fc subunit includes amino acid substitutions 364H and 405A.

In some embodiments, the first Fc subunit includes the amino acid substitution 405L, and the second Fc subunit includes the amino acid substitution 409R.

In some embodiments, the first Fc subunit includes the amino acid substitution 409R, and the second Fc subunit includes the amino acid substitution 405L.

In some embodiments, the first Fc subunit includes amino acid substitutions 366L, 392L, and 394W, and the second Fc subunit includes amino acid substitutions 351Y, 405A, and 407V.

In some embodiments, the first Fc subunit includes amino acid substitutions 351Y, 405A, and 407V, and the second Fc subunit includes amino acid substitutions 366L, 392L, and 394W.

In some embodiments, the first Fc subunit includes amino acid substitutions 360E and 409W, and the second Fc subunit includes amino acid substitutions 347R, 399V, and 405T.

In some embodiments, the first Fc subunit includes amino acid substitutions 347R, 399V, and 405T, and the second Fc subunit includes amino acid substitutions 360E and 409W.

In some embodiments, the first Fc subunit includes amino acid substitutions 349C, 360E, and 409W, and the second Fc subunit includes amino acid substitutions 347R, 354C, 399V, and 405T.

In some embodiments, the first Fc subunit includes amino acid substitutions 347R, 354C, 399V, and 405T, and the second Fc subunit includes amino acid substitutions 349C, 360E, and 409W.

In some embodiments, the first Fc subunit includes amino acid substitutions 370E and 409W, and the second Fc subunit includes amino acid substitutions 357N, 399V, and 405T.

In some embodiments, the first Fc subunit includes amino acid substitutions 357N, 399V, and 405T, and the second Fc subunit includes amino acid substitutions 370E and 409W.

In some embodiments, the first Fc subunit includes amino acid substitutions 360D, 399M, and 407A, and the second Fc subunit includes amino acid substitutions 345R, 347R, 366V, and 409V.

In some embodiments, the first Fc subunit includes amino acid substitutions 345R, 347R, 366V, and 409V, and the second Fc subunit includes amino acid substitutions 360D, 399M, and 407A.

In some embodiments, the first Fc subunit includes amino acid substitutions 349S, 366M, 370Y, and 409V, and the second Fc subunit includes amino acid substitutions 356G, 357D, 364Q, and 407A.

In some embodiments, the first Fc subunit includes amino acid substitutions 356G, 357D, 364Q, and 407A, and the second Fc subunit includes amino acid substitutions 349S, 366M, 370Y, and 409V.

In some embodiments, the first Fc subunit includes amino acid substitutions 368D and 370S, and the second Fc subunit includes amino acid substitutions 356Q and 364K.

In some embodiments, the first Fc subunit includes amino acid substitutions 356Q and 364K, and the second Fc subunit includes amino acid substitutions 368D and 370S.

In some embodiments, the first Fc subunit includes the amino acid substitution 366Y, and the second Fc subunit includes amino acid substitutions 366S, 368A, and 407T.

In some embodiments, the first Fc subunit includes amino acid substitutions 366S, 368A, and 407T, and the second Fc subunit includes the amino acid substitution 366Y.

In some embodiments, the first Fc subunit includes a first charged region, and the second Fc subunit includes a second charged region, and in which the first charged region forms a charge pair with the second charged region. In some embodiments, the first charged region includes a basic amino acid residue, and the second charged region includes an acidic amino acid residue. In some embodiments, the first charged region includes an acidic amino acid residue, and the second charged region includes a basic amino acid residue.

In some embodiments, the first Fc subunit and the second Fc subunit include amino acid substitutions: (a) K409D in the first Fc subunit; and D399K in the second Fc subunit; (b) D399K in the first Fc subunit; and K409D in the second Fc subunit; (c) K390D and K409D in the first Fc subunit; and E356K and D399K in the second Fc subunit; (d) E356K and D399K in the first Fc subunit; and K390D and K409D in the second Fc subunit; (e) K390D and K409D in the first Fc subunit; and E357K and D399K in the second Fc subunit; (f) E357K and D399K in the first Fc subunit; and K390D and K409D in the second Fc subunit; (g) K370D and K409D in the first Fc subunit; and E357K and D399K in the second Fc subunit; (h) E357K and D399K in the first Fc subunit; and K370D and K409D in the second Fc subunit; (i) R392D and K409D in the first Fc subunit; and E356K and D399K in the second Fc subunit; (j) E356K and D399K in the first Fc subunit; and R392D and K409D in the second Fc subunit; (k) L351 D and L368E in the first Fc subunit; and L351K and T366K in the second Fc subunit; or (l) L351K and T366K in the first Fc subunit; and L351 D and L368E in the second Fc subunit, in which the amino acid positions are based on EU numbering.

In some embodiments, the first Fc subunit includes the amino acid substitution K409D, and the second Fc subunit includes the amino acid substitution D399K.

In some embodiments, the first Fc subunit includes the amino acid substitution D399K, and the second Fc subunit includes the amino acid substitution K409D.

In some embodiments, the first Fc subunit includes amino acid substitutions K390D and K409D, and the second Fc subunit includes amino acid substitutions E356K and D399K.

In some embodiments, the first Fc subunit includes amino acid substitutions E356K and D399K, and the second Fc subunit includes amino acid substitutions K390D and K409D.

In some embodiments, the first Fc subunit includes amino acid substitutions K390D and K409D, and the second Fc subunit includes amino acid substitutions E357K and D399K.

In some embodiments, the first Fc subunit includes amino acid substitutions E357K and D399K, and the second Fc subunit includes amino acid substitutions K390D and K409D.

In some embodiments, the first Fc subunit includes amino acid substitutions K370D and K409D, and the second Fc subunit includes amino acid substitutions E357K and D399K.

In some embodiments, the first Fc subunit includes amino acid substitutions E357K and D399K, and the second Fc subunit includes amino acid substitutions K370D and K409D.

In some embodiments, the first Fc subunit includes amino acid substitutions R392D and K409D, and the second Fc subunit includes amino acid substitutions E356K and D399K.

In some embodiments, the first Fc subunit includes amino acid substitutions E356K and D399K, and the second Fc subunit includes amino acid substitutions R392D and K409D.

In some embodiments, the first Fc subunit includes amino acid substitutions L351 D and L368E, and the second Fc subunit includes amino acid substitutions L351K and T366K.

In some embodiments, the first Fc subunit includes amino acid substitutions L351K and T366K, and the second Fc subunit includes amino acid substitutions L351 D and L368E.

In some embodiments, the first Fc subunit or the second Fc subunit includes the amino acid substitutions 435R and 436F, in which the amino acid positions are based on EU numbering. In some embodiments, the first Fc subunit includes the amino acid substitutions 435R and 436F. In some embodiments, the second Fc subunit includes the amino acid substitutions 435R and 436F.

In some embodiments, the first Fc subunit and the second Fc subunit include at least one of (e.g., one, two, three, four, five, six, seven, or all eight of) the following amino acid substitutions: (a) 252Y and, optionally, at least one amino acid substitution selected from the group consisting of 251 D or 251 E; 285N or 285D; 286D, 286Y, 286F, 286L, or 286W; 307Q; 308P; 315D; 426L, 426H, 426F, or 426Y; 430A or 430K; 433K; 435Y; and 436H; (b) 252M and, optionally, at least one amino acid substitution selected from the group consisting of 251 D or 251 E; 256D or 256F; 285N or 285D; 286D, 286Y, 286F, 286L, or 286W; 307Q; 308P; 315D; 426L, 426H, 426F, or 426Y; 430A or 430K; 433K; 435Y; and 436H; (c) 434H and, optionally, at least one amino acid substitution selected from the group consisting of 286Y, 286F, 286L, or 286W; and 426Y, 426F, 426L, or 426W; (d) 434R; (e) 426Y and, optionally, at least one amino acid substitution selected from the group consisting of 286F, 286W, 286L, or 286Y; 312P; 434R; and 436H; (f) 426H and, optionally, at least one amino acid substitution selected from the group consisting of 286F, 286W, 286L, or 286Y; 312P; 434R; and 436H; (g) 426F and, optionally, at least one amino acid substitution selected from the group consisting of 286F, 286W, 286L, or 286Y; 312P; 434R; and 426H; or (h) 434R and, optionally, at least one amino acid substitution selected from the group consisting of 286L; 286Y; 312P; and 436H, in which the amino acid positions are based on EU numbering. In some embodiments, the first Fc subunit and the second Fc subunit include the amino acid substitutions 426Y and 286L.

In some embodiments, the first Fc subunit and the second Fc subunit include the amino acid substitutions 234A and 235A, in which the amino acid positions are based on EU numbering.

In some embodiments, the first Fc subunit includes the amino acid substitutions 234A, 235A, 286L, 354C, 366W, and 426Y; and the second Fc subunit includes the amino acid substitutions 234A, 235A, 286L, 349C, 366S, 368A, 407V, 426Y, 435R, and 436F, in which the amino acid positions are based on EU numbering.

In some embodiments, the first Fc subunit and the second Fc subunit are variants of wild type canine IgGA, IgGB, IgGC, or IgGD Fc. In some embodiments, the first Fc subunit and the second Fc subunit include a sequence having at least 80% identity (e.g., 85%, 90%, 95%, 97%, 99%, or 100% identity) to any one of SEQ ID NOs: 41-44.

In some embodiments, the first Fc subunit and the second Fc subunit are variants of wild type canine IgGA Fc. In some embodiments, the first Fc subunit and the second Fc subunit include a sequence having at least 80% identity (e.g., 85%, 90%, 95%, 97%, 99%, or 100% identity) to SEQ ID NO: 41.

In some embodiments, the first Fc subunit and the second Fc subunit are variants of wild type canine IgGB Fc. In some embodiments, the first Fc subunit and the second Fc subunit include a sequence having at least 80% identity (e.g., 85%, 90%, 95%, 97%, 99%, or 100% identity) to SEQ ID NO: 42.

In some embodiments, the first Fc subunit and the second Fc subunit are variants of wild type canine IgGC Fc. In some embodiments, the first Fc subunit and the second Fc subunit include a sequence having at least 80% identity (e.g., 85%, 90%, 95%, 97%, 99%, or 100% identity) to SEQ ID NO: 43.

In some embodiments, the first Fc subunit and the second Fc subunit are variants of wild type canine IgGD Fc. In some embodiments, the first Fc subunit and the second Fc subunit include a sequence having at least 80% identity (e.g., 85%, 90%, 95%, 97%, 99%, or 100% identity) to SEQ ID NO: 44.

In some embodiments, the first CH1 is linked to the first Fc subunit by a first hinge region or a first linker sequence, and the second CL is linked to the second Fc subunit by a second hinge region or a second linker sequence. In some embodiments, the first CH1 is linked at the C-terminus to the N-terminus of the first hinge region or the first linker sequence, and the first hinge region or the first linker sequence is linked at the C-terminus to the N-terminus of the first Fc subunit. In some embodiments, the second CL is linked at the C-terminus to the N-terminus of the second hinge region or the second linker sequence, and the second hinge region or the second linker sequence is linked at the C-terminus to the N-terminus of the second Fc subunit.

In some embodiments, the first CH1 is linked to the first Fc subunit by a first hinge region, and the second CL is linked to the second Fc subunit by a second hinge region. In some embodiments, the first CH1 is linked at the C-terminus to the N-terminus of the first hinge region, and the first hinge region is linked at the C-terminus to the N-terminus of the first Fc subunit. In some embodiments, the second CL is linked at the C-terminus to the N-terminus of the second hinge region, and the second hinge region is linked at the C-terminus to the N-terminus of the second Fc subunit.

In some embodiments, the first CH1 is linked to the first Fc subunit by a first linker sequence, and the second CL is linked to the second Fc subunit by a second linker sequence. In some embodiments, the first CH1 is linked at the C-terminus to the N-terminus of the first linker sequence, and the first linker sequence is linked at the C-terminus to the N-terminus of the first Fc subunit. In some embodiments, the second CL is linked at the C-terminus to the N-terminus of the second linker sequence, and the second linker sequence is linked at the C-terminus to the N-terminus of the second Fc subunit.

In some embodiments, the first hinge region and the second hinge region include a sequence of:

(a)
(SEQ ID NO: 35)
VPKRENGRVPRPPDCPKCP;
(b)
(SEQ ID NO: 34)
VFNECRCTDTPPCP;
(c)
(SEQ ID NO: 45)
PKRENGRVPRPPDCPKCP;
(d)
(SEQ ID NO: 46)
FNECRCTDTPPCPVPEP;
(e)
(SEQ ID NO: 47)
PKRENGRVPRPPDCPKCPAPEM;
(f)
(SEQ ID NO: 48)
AKECECKCNCNNCPCPGCGL;
(g)
(SEQ ID NO: 49)
PKESTCKCISPCPVPES;
(h)
(SEQ ID NO: 50)
PKESTCKCIPPCPVPES;
(i)
(SEQ ID NO: 51)
KTDHPPGPKPCDCPKCP;
or
(j)
(SEQ ID NO: 52)
KTASTIESKTGEGPKCP.

In some embodiments, a linker sequence may be used instead of a hinge region to connect the polypeptide to the Fc subunit. In some embodiments, the linker is made up of from 1 to 20 amino acids linked by peptide bonds, in which the amino acids are selected from the 20 naturally occurring amino acids. Some of these amino acids may be glycosylated, as is well understood by those in the art. In some embodiments, the 1 to 20 amino acids are each independently selected from glycine, alanine, proline, asparagine, glutamine, and lysine. In some embodiments, a linker is made up of a majority (e.g., more than 50% of the amino acid residues) of amino acids that are sterically unhindered, such as glycine and alanine. Examples of peptide linkers include: Gly, Ser; Gly Ser; Gly Gly Ser; Ser Gly Gly; Gly Gly Gly Ser (SEQ ID NO: 393); Ser Gly Gly Gly (SEQ ID NO: 394); Gly Gly Gly Gly Ser (SEQ ID NO: 395); Ser Gly Gly Gly Gly (SEQ ID NO: 396); Gly Gly Gly Gly Gly Ser (SEQ ID NO: 397); Ser Gly Gly Gly Gly Gly (SEQ ID NO: 398); Gly Gly Gly Gly Gly Gly Ser (SEQ ID NO: 399); Ser Gly Gly Gly Gly Gly Gly (SEQ ID NO: 400); Gly Pro Gly Gly Gln (SEQ ID NO: 53); (Gly Gly Gly Gly Ser)ₙ (SEQ ID NO: 395), wherein n is an integer of one or more (e.g., 1, 2, 3, 4, 5); and (Ser Gly Gly Gly Gly)ₙ (SEQ ID NO: 396), wherein n is an integer of one or more (e.g., 1, 2, 3, 4, 5).

The hinge region, if used, in an antibody (e.g., a bispecific antibody, an antibody, or an antigen-binding fragment thereof) of this disclosure, may include zero to six (i.e., 0, 1, 2, 3, 4, 5, or 6) amino acid substitutions relative to an amino acid sequence set forth in any one of SEQ ID NOs: 34, 35, and 45-52. In some embodiments, the hinge region used in a recombinant protein of this disclosure is at least 80%, at least 85%, at least 90%, at least 95%, at least 96%, at least 97%, at least 98%, at least 99%, or 100% identical to an amino acid sequence set forth in any one of SEQ ID NOs: 34, 35, and 45-52.

Non-peptide linkers may also be used to link a polypeptide or polypeptides of interest to an Fc domain variant disclosed herein. For example, alkyl linkers such as —NH(CH₂)ₙC(O)—, in which n=2-20 can be used. These alkyl linkers may further be substituted by any non-sterically hindering group such as lower alkyl (e.g., C₁-C₆) lower acyl, halogen (e.g., Cl, Br), CN, NH2, phenyl, and the like.

In some embodiments, the first hinge region and the second hinge region include the sequence of VPKRENGRVPRPPDCPKCP (SEQ ID NO: 35). In some embodiments, the first hinge region and the second hinge region include the sequence of VFNECRCTDTPPCP (SEQ ID NO: 34).

In some embodiments, the first light chain includes a sequence having at least 80% identity (e.g., 85%, 90%, 95%, 97%, 99%, or 100% identity) to SEQ ID NO: 28 or 29. In some embodiments, the first light chain includes the sequence of SEQ ID NO: 28 or 29. In some embodiments, the first light chain includes a sequence having at least 80% identity (e.g., 85%, 90%, 95%, 97%, 99%, or 100% identity) to SEQ ID NO: 28. In some embodiments, the first light chain includes the sequence of SEQ ID NO: 28. In some embodiments, the first light chain includes a sequence having at least 80% identity (e.g., 85%, 90%, 95%, 97%, 99%, or 100% identity) to SEQ ID NO: 29. In some embodiments, the first light chain includes the sequence of SEQ ID NO: 29.

In some embodiments, the first heavy chain includes a sequence having at least 80% identity (e.g., 85%, 90%, 95%, 97%, 99%, or 100% identity) to SEQ ID NO: 30. In some embodiments, the first heavy chain includes the sequence of SEQ ID NO: 30.

In some embodiments, the second light chain includes a sequence having at least 80% identity (e.g., 85%, 90%, 95%, 97%, 99%, or 100% identity) to any one of SEQ ID NOs: 1-3 and 12-15. In some embodiments, the second light chain includes a sequence of any one of SEQ ID NOs: 1-3 and 12-15. In some embodiments, the second light chain includes a sequence having at least 80% identity (e.g., 85%, 90%, 95%, 97%, 99%, or 100% identity) to SEQ ID NO: 14 or 15. In some embodiments, the second light chain includes the sequence of SEQ ID NO: 14 or 15. In some embodiments, the second light chain includes a sequence having at least 80% identity (e.g., 85%, 90%, 95%, 97%, 99%, or 100% identity) to SEQ ID NO: 14. In some embodiments, the second light chain includes the sequence of SEQ ID NO: 14.

In some embodiments, the second light chain includes a sequence having at least 80% identity (e.g., 85%, 90%, 95%, 97%, 99%, or 100% identity) to SEQ ID NO: 15. In some embodiments, the second light chain includes the sequence of SEQ ID NO: 15. In some embodiments, the second light chain includes a sequence having at least 80% identity (e.g., 85%, 90%, 95%, 97%, 99%, or 100% identity) to SEQ ID NO: 1. In some embodiments, the second light chain includes the sequence of SEQ ID NO: 1. In some embodiments, the second light chain includes a sequence having at least 80% identity (e.g., 85%, 90%, 95%, 97%, 99%, or 100% identity) to SEQ ID NO: 2. In some embodiments, the second light chain includes the sequence of SEQ ID NO: 2. In some embodiments, the second light chain includes a sequence having at least 80% identity (e.g., 85%, 90%, 95%, 97%, 99%, or 100% identity) to SEQ ID NO: 3. In some embodiments, the second light chain includes the sequence of SEQ ID NO: 3. In some embodiments, the second light chain includes a sequence having at least 80% identity (e.g., 85%, 90%, 95%, 97%, 99%, or 100% identity) to SEQ ID NO: 12. In some embodiments, the second light chain includes the sequence of SEQ ID NO: 12. In some embodiments, the second light chain includes a sequence having at least 80% identity (e.g., 85%, 90%, 95%, 97%, 99%, or 100% identity) to SEQ ID NO: 13. In some embodiments, the second light chain includes the sequence of SEQ ID NO: 13.

In some embodiments, the second heavy chain includes a sequence having at least 80% identity (e.g., 85%, 90%, 95%, 97%, 99%, or 100% identity) to any one of SEQ ID NOs: 4-11. In some embodiments, the second heavy chain includes a sequence of any one of SEQ ID NOs: 4-11. In some embodiments, the second heavy chain includes a sequence having at least 80% identity (e.g., 85%, 90%, 95%, 97%, 99%, or 100% identity) to SEQ ID NO: 5, 8, or 10. In some embodiments, the second heavy chain includes the sequence of SEQ ID NO: 5, 8, or 10. In some embodiments, the second heavy chain includes a sequence having at least 80% identity (e.g., 85%, 90%, 95%, 97%, 99%, or 100% identity) to SEQ ID NO: 10. In some embodiments, the second heavy chain includes the sequence of SEQ ID NO: 10. In some embodiments, the second heavy chain includes a sequence having at least 80% identity (e.g., 85%, 90%, 95%, 97%, 99%, or 100% identity) to SEQ ID NO: 5. In some embodiments, the second heavy chain includes the sequence of SEQ ID NO: 5. In some embodiments, the second heavy chain includes a sequence having at least 80% identity (e.g., 85%, 90%, 95%, 97%, 99%, or 100% identity) to SEQ ID NO: 8. In some embodiments, the second heavy chain includes the sequence of SEQ ID NO: 8. In some embodiments, the second heavy chain includes a sequence having at least 80% identity (e.g., 85%, 90%, 95%, 97%, 99%, or 100% identity) to SEQ ID NO: 4. In some embodiments, the second heavy chain includes the sequence of SEQ ID NO: 4. In some embodiments, the second heavy chain includes a sequence having at least 80% identity (e.g., 85%, 90%, 95%, 97%, 99%, or 100% identity) to SEQ ID NO: 6. In some embodiments, the second heavy chain includes the sequence of SEQ ID NO: 6. In some embodiments, the second heavy chain includes a sequence having at least 80% identity (e.g., 85%, 90%, 95%, 97%, 99%, or 100% identity) to SEQ ID NO: 7. In some embodiments, the second heavy chain includes the sequence of SEQ ID NO: 7. In some embodiments, the second heavy chain includes a sequence having at least 80% identity (e.g., 85%, 90%, 95%, 97%, 99%, or 100% identity) to SEQ ID NO: 9. In some embodiments, the second heavy chain includes the sequence of SEQ ID NO: 9. In some embodiments, the second heavy chain includes a sequence having at least 80% identity (e.g., 85%, 90%, 95%, 97%, 99%, or 100% identity) to SEQ ID NO: 11. In some embodiments, the second heavy chain includes the sequence of SEQ ID NO: 11.

In some embodiments, the first light chain includes a sequence having at least 80% identity (e.g., 85%, 90%, 95%, 97%, 99%, or 100% identity) to SEQ ID NO: 28 or 29; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to any one of SEQ ID NOs: 1-3 and 12-15; and the second heavy chain includes a sequence having at least 80% identity (e.g., 85%, 90%, 95%, 97%, 99%, or 100% identity) to any one of SEQ ID NOs: 4-11.

In some embodiments, the first light chain includes the sequence of SEQ ID NO: 28 or 29; the first heavy chain includes the sequence of SEQ ID NO: 30; the second light chain includes a sequence of any one of SEQ ID NOs: 1-3 and 12-15; and the second heavy chain includes a sequence of any one of SEQ ID NOs: 4-11.

In some embodiments, the first light chain includes a sequence having at least 80% identity (e.g., 85%, 90%, 95%, 97%, 99%, or 100% identity) to SEQ ID NO: 28; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to SEQ ID NO: 14 or 15; and the second heavy chain includes a sequence having at least 80% identity (e.g., 85%, 90%, 95%, 97%, 99%, or 100% identity) to SEQ ID NO: 5, 8, or 10.

In some embodiments, the first light chain includes the sequence of SEQ ID NO: 28; the first heavy chain includes the sequence of SEQ ID NO: 30; the second light chain includes the sequence of SEQ ID NO: 14 or 15; and the second heavy chain includes the sequence of SEQ ID NO: 5, 8, or 10.

In some embodiments, the first light chain includes a sequence having at least 80% identity (e.g., 85%, 90%, 95%, 97%, 99%, or 100% identity) to SEQ ID NO: 28; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to SEQ ID NO: 14; and the second heavy chain includes a sequence having at least 80% identity (e.g., 85%, 90%, 95%, 97%, 99%, or 100% identity) to SEQ ID NO: 5.

In some embodiments, the first light chain includes the sequence of SEQ ID NO: 28; the first heavy chain includes the sequence of SEQ ID NO: 30; the second light chain includes the sequence of SEQ ID NO: 14; and the second heavy chain includes the sequence of SEQ ID NO: 5.

In some embodiments, the first light chain includes a sequence having at least 80% identity (e.g., 85%, 90%, 95%, 97%, 99%, or 100% identity) to SEQ ID NO: 28; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to SEQ ID NO: 14; and the second heavy chain includes a sequence having at least 80% identity (e.g., 85%, 90%, 95%, 97%, 99%, or 100% identity) to SEQ ID NO: 8.

In some embodiments, the first light chain includes the sequence of SEQ ID NO: 28; the first heavy chain includes the sequence of SEQ ID NO: 30; the second light chain includes the sequence of SEQ ID NO: 14; and the second heavy chain includes the sequence of SEQ ID NO: 8.

In some embodiments, the first light chain includes a sequence having at least 80% identity (e.g., 85%, 90%, 95%, 97%, 99%, or 100% identity) to SEQ ID NO: 28; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to SEQ ID NO: 15; and the second heavy chain includes a sequence having at least 80% identity (e.g., 85%, 90%, 95%, 97%, 99%, or 100% identity) to SEQ ID NO: 5.

In some embodiments, the first light chain includes the sequence of SEQ ID NO: 28; the first heavy chain includes the sequence of SEQ ID NO: 30; the second light chain includes the sequence of SEQ ID NO: 15; and the second heavy chain includes the sequence of SEQ ID NO: 5.

In some embodiments, the first light chain includes a sequence having at least 80% identity (e.g., 85%, 90%, 95%, 97%, 99%, or 100% identity) to SEQ ID NO: 28; the first heavy chain includes a sequence having at least 80% identity (e.g., 85%, 90%, 95%, 97%, 99%, or 100% identity) to SEQ ID NO: 30; the second light chain includes a sequence having at least 80% identity (e.g., 85%, 90%, 95%, 97%, 99%, or 100% identity) to SEQ ID NO: 15; and the second heavy chain includes a sequence having at least 80% identity (e.g., 85%, 90%, 95%, 97%, 99%, or 100% identity) to SEQ ID NO: 8.

In some embodiments, the first light chain includes the sequence of SEQ ID NO: 28; the first heavy chain includes the sequence of SEQ ID NO: 30; the second light chain includes the sequence of SEQ ID NO: 15; and the second heavy chain includes the sequence of SEQ ID NO: 8.

### Antigen-Binding Moiety of Bispecific Antibodies

The bispecific antibodies of this disclosure may include two or more antigen-binding moieties. In some embodiments, the bispecific antibodies of this disclosure include a first antigen-binding moiety that specifically binds to a first antigen and a second antigen-binding moiety that specifically binds to a second antigen. An antigen-binding moiety can specifically bind to a protein, subunit, domain, motif, and/or epitope of a selected antigen described herein.

In some embodiments, the antigen-binding moiety comprises an antibody, an antibody fragment, or a ligand-binding portion of a receptor. In some embodiments, the antibody or the antibody fragment comprises six complementarity determining regions (CDRs) of an immunoglobulin molecule. In some embodiments, the antibody fragment is selected from the group consisting of Fab, single chain variable fragment (scFv), Fv, Fab′, Fab′-SH, F(ab′)₂, nanobody, and diabody. In some embodiments, the antibody or the antibody fragment comprises a single-domain antibody. In some embodiments, the single-domain antibody is a VHH domain. In other embodiments, the ligand-binding portion of a receptor comprises a ligand binding domain of a receptor protein or an extracellular domain of a receptor protein.

In some embodiments, the antigen that is specifically bound by an antigen-binding moiety of this disclosure is selected from the group consisting of nerve growth factor (NGF), Activin receptor type IIA (RIIA), Activin receptor type IIB (RIIB), TrKA, ADAMTS, IL-1, IL-2, IL-4, IL-4R, angiotensin type 1 (AT1) receptor, angiotensin type 2 (AT2) receptor, IL-5, IL-12, IL-13, IL-31, IL-31R, IL-33, CD3, CD20, CD47, CD52, and a complement system component. In some embodiments, the complement system component is selected from the group consisting of a complement C1 complex component (e.g., C1 q, C1 r, and C1s), C2, C3, C4, C5, C6, C7, C8, C9, Factor H, Factor I, CD55, CD59, Factor B, Factor D, properdin, mannose-binding lectin, ficolin, and mannan-binding lectin serine protease (MASP) (e.g., MASP-1, MASP-2, and MASP-3). In some embodiments, the antigen is NGF. In some embodiments, the antigen is Activin RIIA. In some embodiments, the antigen is Activin RIIB. In some embodiments, the antigen is IL-4R. In some embodiments, the antigen is IL-31. In some embodiments, the antigen is IL-31R.

In some embodiments, the antigen that is specifically bound by an antigen-binding moiety of this disclosure is selected from the group consisting of 17-IA, 4-1 BB, 4Dc, 6-keto-PGF1 a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, IgE, Angiotensin type 1 (AT1) receptor, Angiotensin type 2 (AT2) receptor, ARC, ART, Artemin, anti-Id, ASPARTIC, Atrial natriuretic factor, av/b3 integrin, Axl, b2M, R7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11 a, CD11 b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD47, CD49a, CD52, CD54, CD55, CD56, CD61, CD64, CD66e, CD74, CD80 (R7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxini, EpCAM, Ephrin B2/EphB4, EPO, ERCC, E-selectin, ET-1, Factor Ila, Factor VII, Factor VIlIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alphai, GFR-alpha2, GFR-alpha3, GITR, GLP1, GLP2, Glucagon, Glut 4, glycoprotein IIb/IIIa (GP IIb/IIIa), GM-CSF, gp130, gp72, GRO, GnRH, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, cardiac myosin, cytomegalovirus (CMV), growth hormone (GH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/betai, integrin alpha4/beta7, integrin alpha5 (alphaV), integrin alpha5/betai, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpi, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucd), MUC18, Muellerian-inhibitin substance, Mug, MuSK, NAIP, NAP, NAV 1.7, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, oncostatin M receptor (OSMR), OPG, OPN, OSM, OX40L, OX40R, p150, p95, PADPr, Parathyroid hormone, PARC, PARP, PBR, PBSF, PCAD, P-Cadherin, PCNA, PD1, PDL1,PDGF, PDGF, PDK-1, PECAM, PEM, PF4, PGE, PGF, PG12, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1 BB CD137, ILA), TNFRSF21 (DR6), TNFRSF22 (DCTRAIL R2 TNFRH2), TNFRST23 (DCTRAIL RiTNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk (e.g., TrkA), TROP-2, TSG, TSLP, tumor-associated antigen CA 125, tumor-associated antigen expressing Lewis Y related carbohydrate, TWEAK, TXB2, Ung, UPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factor.

In some embodiments, the antigen-binding moiety of this disclosure includes one or more complementarity determining regions (CDRs) with amino acid sequences selected from Table 2 below. For example, the antigen-binding moiety may include a CDR-H1, a CDR-H2, a CDR-H3, a CDR-L1, a CDR-L2, and a CDR-L3 selected from Table 2. For example, the antigen-binding moiety may include all six CDRs for an antibody that is listed as binding to a particular antigen in Table 2. In some embodiments, the antigen-binding moiety may be disclosed in, or may be derived from any antibody or antibody fragment disclosed in, U.S. Patent Application Publication Nos. US 2020/0062840, US 2022/0251209, US 2021/0040223, US 2022/0204615, US 2022/0251230, US 2021/0163618, US 2021/0253722, US 2022/0119513, US 2022/0106391, or US 2022/0177594; U.S. Pat. Nos. 11,091,556, 11,447,561, 10,040,849, or U.S. Pat. No. 9,951,128; and International Patent Application Publication Nos. WO 2020/056393, WO 2022/079138, WO 2021/123092, WO 2022/029447, or WO 2022/133325.

TABLE 2
Exemplary CDR sequences for canine antibodies

| Antigen | Publication Number | Sequences | SEQ ID NO: |
|---|---|---|---|
| IL-31 | US 2020/0062840 | CDR-H1: GDSITSGYW | 84 |
| | | CDR-H2: YISYSGITDYNPSLKS | 85 |
| | | CDR-H3: ARYGNYGYAMDY | 86 |
| | | CDR-L1: RASESVDTYGNSFMH | 87 |
| | | CDR-L2: RASNLES | 88 |
| | | CDR-L3: QQSYEDPWT | 89 |
| IL-31 | US 2020/0062840 | CDR-H1: GDSITSGYW | 90 |
| | | CDR-H2: YISYSGITYYNPSLKS | 91 |
| | | CDR-H3: ARYGNYGYAMDY | 92 |
| | | CDR-L1: RASESVDTYGNSFIH | 93 |
| | | CDR-L2: RASNLES | 94 |
| | | CDR-L3: QQSYEDPWT | 95 |
| IL-31 | US 2020/0062840 | CDR-H1: GDSITGYW | 96 |
| | | CDR-H2: YISYSGITDYNPSLKS | 97 |
| | | CDR-H3: ARYGNYGYAMDY | 98 |
| | | CDR-L1: RASESVDTYGNSFMH | 99 |
| | | CDR-L2: RASNLES | 100 |
| | | CDR-L3: QQSYEDPWT | 101 |
| IL-4R | WO 2020/056393 | CDR-H1: GYTFTSYVMH | 102 |
| | | CDR-H2: YINPKNDGTFYNGKFKG | 103 |
| | | CDR-H3: FNYGIAY | 104 |
| | | CDR-L1: RASQEISGYLS | 105 |
| | | CDR-L2: AASTLDS | 106 |
| | | CDR-L3: VQYASYPWT | 107 |
| IL-4R | WO 2020/056393 | CDR-H1: GYTFTSYVMH | 108 |
| | | CDR-H2: YINPNNDGTFYNGKFKG | 109 |
| | | CDR-H3: FYYGFAY | 110 |
| | | CDR-L1: RASQEISGYLS | 111 |
| | | CDR-L2: AASTLDS | 112 |
| | | CDR-L3: LQYASYPWT | 113 |
| IL-4R | WO 2020/056393 | CDR-H1: TVSGFSLSRYSVH | 114 |
| | | CDR-H2: GMIWGGGST | 115 |
| | | CDR-H3: TYRGYALDY | 116 |
| | | CDR-L1: RASQDISNYLN | 117 |
| | | CDR-L2: YYTSRLQS | 118 |
| | | CDR-L3: QQANTLPLT | 119 |
| IL-4R | WO 2020/056393 | CDR-H1: AASGFTFSSFGMH | 120 |
| | | CDR-H2: AYIRSDSSTIY | 121 |
| | | CDR-H3: SGYYGSFSLTY | 122 |
| | | CDR-L1: SASSSVSSNYLH | 123 |
| | | CDR-L2: YRTSNLPS | 124 |
| | | CDR-L3: QQGSGMLT | 125 |
| IL-31R | WO 2022/079138 | CDR-H1: NYYMA | 126 |
| | | CDR-H2: SISTGGGNTYYRDSVKG | 127 |
| | | CDR-H3: HGTLYFDY | 128 |
| | | CDR-L1: ERSSGDIGDSYVS | 129 |
| | | CDR-L2: VDDQRPS | 130 |
| | | CDR-L3: QSYDSNIDGPV | 131 |
| IL-31R | WO 2022/079138 | CDR-H1: DYYMA | 132 |
| | | CDR-H2: TISYDGSSTYYRDSVRG | 133 |
| | | CDR-H3: GPLTDWAPNWFAY | 134 |
| | | CDR-L1: QTSEDIYSGLA | 135 |
| | | CDR-L2: GASRLED | 136 |
| | | CDR-L3: QQGLKYPNT | 137 |
| IL-31R | WO 2022/079138 | CDR-H1: SNGVS | 138 |
| | | CDR-H2: AISSGGSTYYNSVLKS | 139 |
| | | CDR-H3: RLSGYNYVPFAY | 140 |
| | | CDR-L1: KASQNIYKHLA | 141 |
| | | CDR-L2: NANSLQT | 142 |
| | | CDR-L3: QQYYSGDT | 143 |
| IL-4R | U.S. Pat. No. 11,091,556 | CDR-H1: DYAMT | 144 |
| | | CDR-H2: SISGSGGNTYYADSVKG | 145 |
| | | CDR-H3: DRLSITIRPRYYGLDV | 146 |
| | | CDR-L1: RSSQSLLYSIGYNYLD | 147 |
| | | CDR-L2: LGSNRAS | 148 |
| | | CDR-L3: MQALQTPYT | 149 |
| PD-L1 | U.S. Pat. No. 11,447,561 | CDR-H1: SYAMS | 150 |
| | | CDR-H2: TISDGGSYTHYPDNLMG | 151 |
| | | CDR-H3: ESYDGYYVAN | 152 |
| | | CDR-L1: RASQSISNNLH | 153 |
| | | CDR-L2: YASQSIS | 154 |
| | | CDR-L3: QQSNSWPQT | 155 |
| PD-L1 | U.S. Pat. No. 11,447,561 | CDR-H1: DYYMN | 156 |
| | | CDR-H2: WIFPGSGATYYNERFMG | 157 |
| | | CDR-H3: SDWDVGDF | 158 |
| | | CDR-L1: RSSRSLLHTNGITYLS | 159 |
| | | CDR-L2: QMSNLAS | 160 |
| | | CDR-L3: AQTLGLPRT | 161 |
| CTLA-4 | US 2022/0251209 | CDR-H1: DYNMD | 162 |
| | | CDR-H2: NINPNSESTSYNQKFKG | 163 |
| | | CDR-H3: DGNRYDAWFAY | 164 |
| | | CDR-L1: SASSSVTYMH | 165 |
| | | CDR-L2: STSILAS | 166 |
| | | CDR-L3: QQRTSYPLT | 167 |
| IL-4R | US 2021/0040223 | CDR-H1: NYWIH | 168 |
| | | CDR-H2: RIDPNSGGTKYNEKFKS | 169 |
| | | CDR-H3: FGSTYGFAY | 170 |
| | | CDR-L1: KSSQSLLNSRTRKNYLA | 171 |
| | | CDR-L2: WASTRES | 172 |
| | | CDR-L3: KQSYNLYT | 173 |
| IL-4R | US 2022/0204615 | CDR-H1: GYTFTTYGMS | 174 |
| | | CDR-H2: WINIYSGIPTYADDFKG | 175 |
| | | CDR-H3: FDGPDY | 176 |
| | | CDR-L1: KSSQSLLNSVNQKNYLA | 177 |
| | | CDR-L2: FASARVS | 178 |
| | | CDR-L3: QQYFSTPLT | 179 |
| IL-4R | US 2022/0204615 | CDR-H1: GFNIKNTYMH | 180 |
| | | CDR-H2: RIAPANVDTKYAPKFQG | 181 |
| | | CDR-H3: IYYDYDGDIDV | 182 |
| | | CDR-L1: HASQNINVWLS | 183 |
| | | CDR-L2: KASHLHT | 184 |
| | | CDR-L3: QQGQSWPLT | 185 |
| IL-4R | US 2022/0204615 | CDR-H1: GYTFTRYNMH | 186 |
| | | CDR-H2: TIYPGYGDTSYNQKFKG | 187 |
| | | CDR-H3: EFADDYPIPPFDY | 188 |
| | | CDR-L1: RSSQNIVHSNGNTYLE | 189 |
| | | CDR-L2: KVSNRFS | 190 |
| | | CDR-L3: FQGSHVPYT | 191 |
| IL-4R | US 2022/0204615 | CDR-H1: GFSLTSYGVH | 192 |
| | | CDR-H2: WINIYSGIPTYADDFKG | 193 |
| | | CDR-H3: FDGPDY | 194 |
| | | CDR-L1: KSSQSLLNSVNQKNYLA | 195 |
| | | CDR-L2: FASTRVS | 196 |
| | | CDR-L3: QQYFSTPLT | 197 |
| IL-4R | US 2022/0204615 | CDR-H1: GYTFTTYGMS | 198 |
| | | CDR-H2: WINIYSGIPTYADDFKG | 199 |
| | | CDR-H3: FDGPDY | 200 |
| | | CDR-L1: KSSQSLLNSVNQKNYLA | 201 |
| | | CDR-L2: FASTRVS | 202 |
| | | CDR-L3: QQYFSTPLT | 203 |
| IL-4R | US 2022/0204615 | CDR-H1: GFNIKNTYMH | 204 |
| | | CDR-H2: RIDPANVNTKYAPKFQG | 205 |
| | | CDR-H3: IFYDYDGDIDV | 206 |
| | | CDR-L1: HASQNINVWLS | 207 |
| | | CDR-L2: KASNLHT | 208 |
| | | CDR-L3: QQGQSYPLT | 209 |
| IL-4R | US 2022/0204615 | CDR-H1: GYTFTTYGMS | 210 |
| | | CDR-H2: WINIYSGMPTYADDFKG | 21 |
| | | CDR-H3: FDGPDY | 212 |
| | | CDR-L1: KSSQSLLNSVNQKNYLA | 213 |
| | | CDR-L2: FASTRIS | 214 |
| | | CDR-L3: QQYFSTPLT | 215 |
| IL-22 | WO 2021/123092 | CDR-H1: NYYMH | 216 |
| | | CDR-H2: WINPYTGSAFYAQKFRG | 217 |
| | | CDR-H3: EPEKFDSDDSDV | 218 |
| | | CDR-L1: TGSSSNIGAGYGVH | 219 |
| | | CDR-L2: GDSNRPS | 220 |
| | | CDR-L3: QSYDNSLSGYV | 22 |
| OX40L | WO 2022/029447 | CDR-H1: GFTFSSY | 222 |
| | | CDR-H2: NTGGTV | 223 |
| | | CDR-H3: CARGYGVFDYW | 224 |
| | | CDR-L1: GGDNIGSKSVH | 225 |
| | | CDR-L2: YDSRRPT | 226 |
| | | CDR-L3: CQVWDNSDRASWVF | 227 |
| OX40L | WO 2022/029447 | CDR-H1: GGSVTSSH | 228 |
| | | CDR-H2: TGG | N/A |
| | | CDR-H3: | 229 |
| | | CARGGGYSGTWKDYYVMDYW | |
| | | CDR-L1: GGDNLGSKSVH | 230 |
| | | CDR-L2: FDTSRPT | 231 |
| | | CDR-L3: CQVWDSSAKASVF | 232 |
| OX40L | WO 2022/029447 | CDR-H1: GFTFSNF | 233 |
| | | CDR-H2: NSGGFN | 234 |
| | | CDR-H3: CARDWDTHLDTNWFYYW | 235 |
| | | CDR-L1: RGDNIGSKSVH | 236 |
| | | CDR-L2: YDSSRPT | 237 |
| | | CDR-L3: CQVWDISAKASVF | 238 |
| OX40L | WO 2022/029447 | CDR-H1: GFTFSYY | 239 |
| | | CDR-H2: NSGGFS | 240 |
| | | CDR-H3: CASESRWGDSYSGMTYW | 241 |
| | | CDR-L1: GGDNIGSKSVH | 242 |
| | | CDR-L2: YDNSRPT | 243 |
| | | CDR-L3: CQVWDSSAKASVF | 244 |
| OX40L | WO 2022/029447 | CDR-H1: GFTFSNY | 245 |
| | | CDR-H2: TSDGIV | 246 |
| | | CDR-H3: CASGLFLVVGGGTFW | 247 |
| | | CDR-L1: GGDNIGSKSVH | 248 |
| | | CDR-L2: SDSSRPT | 249 |
| | | CDR-L3: CQVWDSSAKASVF | 250 |
| OX40L | WO 2022/029447 | CDR-H1: GFTFSSY | 251 |
| | | CDR-H2: ASGGTV | 252 |
| | | CDR-H3: CTRWKGGTFGYGMDYW | 253 |
| | | CDR-L1: GGDNIGSKSVH | 254 |
| | | CDR-L2: YDNNRPA | 255 |
| | | CDR-L3: CQVWDSSAKASVF | 256 |
| OX40L | WO 2022/029447 | CDR-H1: GFTFSNY | 257 |
| | | CDR-H2: TNDGIV | 258 |
| | | CDR-H3: CASGLFLVVGGGTFW | 259 |
| | | CDR-L1: GGDNIGSKSVH | 260 |
| | | CDR-L2: YDSSRPT | 261 |
| | | CDR-L3: CQVWDSSAKASVF | 262 |
| OX40L | WO 2022/029447 | CDR-H1: GFTFSSN | 263 |
| | | CDR-H2: SGDGIY | 264 |
| | | CDR-H3: CATGIYPNAFGYW | 265 |
| | | CDR-L1: GGDNIGSKSVH | 266 |
| | | CDR-L2: YDSSRPT | 267 |
| | | CDR-L3: CQVWDSSANVF | 268 |
| OX40L | WO 2022/029447 | CDR-H1: GFTFSSY | 269 |
| | | CDR-H2: SSGGTF | 270 |
| | | CDR-H3: CASGLFLVVGGGNYW | 271 |
| | | CDR-L1: GGDNIGSKSVH | 272 |
| | | CDR-L2: SDSSRPT | 273 |
| | | CDR-L3: CQVWDSSASVF | 274 |
| OX40L | WO 2022/029447 | CDR-H1: GFTFSNY | 275 |
| | | CDR-H2: NSDGRV | 276 |
| | | CDR-H3: CARWRGGTFGYGMDYW | 277 |
| | | CDR-L1: GGDNIGSKSVH | 278 |
| | | CDR-L2: YDSSRPT | 279 |
| | | CDR-L3: CQVWDSSAKASVF | 280 |
| OX40L | WO 2022/029447 | CDR-H1: GFTFSNY | 281 |
| | | CDR-H2: NSDGRI | 282 |
| | | CDR-H3: CARWRGGTFGYGMDYW | 283 |
| | | CDR-L1: GGDNIGSKSVH | 284 |
| | | CDR-L2: YDSSRPT | 285 |
| | | CDR-L3: CQVWDSSAKASVF | 286 |
| OX40L | WO 2022/029447 | CDR-H1: GFTFHNY | 287 |
| | | CDR-H2: SSDGRF | 288 |
| | | CDR-H3: CANGLFLVLGGENYW | 289 |
| | | CDR-L1: GGDNIGSKSVH | 290 |
| | | CDR-L2: YDSSRPT | 291 |
| | | CDR-L3: CQVWDSSASVF | 292 |
| OX40L | WO 2022/029447 | CDR-H1: GFTFSSN | 293 |
| | | CDR-H2: SGDGSF | 294 |
| | | CDR-H3: CASGIYPNAFGYW | 295 |
| | | CDR-L1: GGDNIGSKSVH | 296 |
| | | CDR-L2: YDSSRPT | 297 |
| | | CDR-L3: CQVRDSSANVF | 298 |
| CD20 | US 2022/0251230 | CDR-H1: DYGML | 299 |
| | | CDR-H2: YISSGSSTIYYADRVKG | 300 |
| | | CDR-H3: GTFAY | 301 |
| | | CDR-L1: RSSQSLIYNNGNTYLH | 302 |
| | | CDR-L2: KVSNRFS | 303 |
| | | CDR-L3: SQSTHVPFT | 304 |
| CD20 | US 2022/0251230 | CDR-H1: DDYMH | 305 |
| | | CDR-H2: WIDPENGHTKYASKFQG | 306 |
| | | CDR-H3: LRHYYGSSYVSPHYY | 307 |
| | | CDR-L1: KASQNVGPNVA | 308 |
| | | CDR-L2: SASYRYS | 309 |
| | | CDR-L3: QQYNNYPYT | 310 |
| CD20 | US 2022/0251230 | CDR-H1: DYYMN | 311 |
| | | CDR-H2: DINPNNGDTSYNQKFKG | 312 |
| | | CDR-H3: GGVLRYPYYYVMDY | 313 |
| | | CDR-L1: RSNKSLLHRNGNTYLY | 314 |
| | | CDR-L2: RMSNLAS | 315 |
| | | CDR-L3: MQHLEFPFT | 316 |
| CD20 | US 2021/0163618 | CDR-H1: KGSGRTFTSYNMH | 317 |
| | | CDR-H2: WVRQMPGKGLEWMG | 318 |
| | | CDR-H3: AIYPLTGDTSYNQKSKL | 319 |
| | | CDR-L1: RASRSVPYIH | 320 |
| | | CDR-L2: YATSALAS | 321 |
| | | CDR-L3: QQWLSNPPT | 322 |
| NGF | U.S. Pat. No. 10,040,849 | CDR-H1: NNNVN | 323 |
| | | CDR-H2: GVWAGGATDYNSALKS | 324 |
| | | CDR-H3: DGGYSSSTLYAMDA | 325 |
| | | CDR-L1: RASEDIYNALA | 326 |
| | | CDR-L2: NTDTLHT | 327 |
| | | CDR-L3: QHYFHYPRT | 328 |
| NGF | U.S. Pat. No. 9,951,128 | CDR-H1: LIGYDIN | 329 |
| | | CDR-H2: MIWGDGTTDYNSALKS | 330 |
| | | CDR-H3: GGYYYGTSYYFDY | 331 |
| | | CDR-L1: RASQDISNHLN | 332 |
| | | CDR-L2: YISRFHS | 333 |
| | | CDR-L3: QQSKTLPYT | 334 |
| NGF | US 2021/0253722 | CDR-H1: TNNIGILG | 335 |
| | | CDR-H2: GNG | N/A |
| | | CDR-H3: QSFDTTLGAHV | 336 |
| | | CDR-L1: GFTFSSHG | 337 |
| | | CDR-L2: INSGGSST | 338 |
| | | CDR-L3: AKESVGGVVEQLVGPHFDY | 339 |
| NGF | US 2021/0253722 | CDR-H1: TMDIDIFG | 340 |
| | | CDR-H2: SDG | N/A |
| | | CDR-H3: QSGDSTLGALAI | 341 |
| | | CDR-L1: GFTFSTYG | 342 |
| | | CDR-L2: ISSGGSST | 343 |
| | | CDR-L3: AGSRYTYAYGGGYEFHF | 344 |
| TGF-β | US 2022/0119513 | CDR-H1: SSWMN | 345 |
| | | CDR-H2: QIYPGDGDTNYNGKFKG | 346 |
| | | CDR-H3: ARHYDGSTDY | 347 |
| | | CDR-L1: RASENIYSNLA | 348 |
| | | CDR-L2: AATNLAD | 349 |
| | | CDR-L3: QHFWGTPYT | 350 |
| TGF-β | US 2022/0119513 | CDR-H1: FSSYGMH | 351 |
| | | CDR-H2: VISYDGSIKYY | 352 |
| | | CDR-H3: TGEYSGYDTDPQYS | 353 |
| | | CDR-L1: RASQGIGDDLG | 354 |
| | | CDR-L2: GTSTLQS | 355 |
| | | CDR-L3: LQDSNYPLT | 356 |
| TGF-β | US 2022/0119513 | CDR-H1: GYIFITY | 357 |
| | | CDR-H2: FPASGS | 358 |
| | | CDR-H3: GDGNYALDAMDY | 359 |
| | | CDR-L1: RASESVDSYGNSFMH | 360 |
| | | CDR-L2: LASNLES | 361 |
| | | CDR-L3: QQNNEDPLT | 362 |
| NGF | US 2022/0106391 | CDR-H1: GFTLTQYG | 363 |
| | | CDR-H2: VIWATGATD | 364 |
| | | CDR-H3: DGWWYATSWYFDV | 365 |
| | | CDR-L1: KASQDINHYLN | 366 |
| | | CDR-L2: YTSRLHS | 367 |
| | | CDR-L3: QQGDHFPRT | 368 |
| OSMR-β | US 2022/0177594 | CDR-H1: SYAMS | 369 |
| | | CDR-H2: YISSGGDYIYYADTVKG | 370 |
| | | CDR-H3: DPITGTFAY | 371 |
| | | CDR-L1: RASQDINNYLN | 372 |
| | | CDR-L2: YTSTLHS | 373 |
| | | CDR-L3: QQGNTLPWT | 374 |
| OSMR-β | US 2022/0177594 | CDR-H1: SYAMS | 375 |
| | | CDR-H2: YISSGGDYFYYADTVKG | 376 |
| | | CDR-H3: DPITGTFAY | 377 |
| | | CDR-L1: RASQDITNYLN | 378 |
| | | CDR-L2: YTSTLHS | 379 |
| | | CDR-L3: QQGHMLPWT | 380 |
| OSMR-β | US 2022/0177594 | CDR-H1: NYWMN | 381 |
| | | CDR-H2: QIYPGHVNTNYNGNFKD | 382 |
| | | CDR-H3: SADNSGFVLFAY | 383 |
| | | CDR-L1: RASKSVSTSGYSYLH | 384 |
| | | CDR-L2: LASNLES | 385 |
| | | CDR-L3: QHSRELPLT | 386 |
| OSMR-β | US 2022/0177594 | CDR-H1: DYYMA | 387 |
| | | CDR-H2: NINYDGSSTYYLDSLKS | 388 |
| | | CDR-H3: GLTWDFDV | 389 |
| | | CDR-L1: KASQDVDTAVA | 390 |
| | | CDR-L2: LASTRHT | 391 |
| | | CDR-L3: QQYSRFPLT | 392 |
| TNF-α | WO 2022/133325 | CDR-H1: FTFDDYAMH | 414 |
| | | CDR-H2: AITWNSGHIDYADSVEGR | 415 |
| | | CDR-H3: AKVSYLSTASSLDY | 416 |
| | | CDR-L1: RASQGIRNYLA | 417 |
| | | CDR-L2: AASTLQ | 418 |
| | | CDR-L3: QRYNRAPYT | 419 |
| TNF-α | WO 2022/133325 | CDR-H1: NHWMN | 420 |
| | | CDR-H2: EIRSKSINSATHYAESVKG | 421 |
| | | CDR-H3: NYYGSTYDY | 422 |
| | | CDR-L1: RASQFVGSSIH | 423 |
| | | CDR-L2: YASESMS | 424 |
| | | CDR-L3: QQSHSWPFT | 425 |
| TNF-α | WO 2022/133325 | CDR-H1: VASGFSLTNNNVN | 426 |
| | | CDR-H2: GVWAGGATD | 427 |
| | | CDR-H3: ARDGGYSSSTLYAMDA | 428 |
| | | CDR-L1: RASEDIYNALA | 429 |
| | | CDR-L2: YNTDTLHT | 430 |
| | | CDR-L3: QHYFGYPRT | 431 |
| NGF | N/A | CDR-H1: GMSLWSNS | 55 |
| | | CDR-H2: IWSQGGT | 56 |
| | | CDR-H3: AQIYYYDAEYLHWYFDF | 57 |
| | | CDR-L1: EGISKN | 58 |
| | | CDR-L2: ATS | N/A |
| | | CDR-L3: QQGYQFPLT | 59 |
| Activin | N/A | CDR-H1: GYSFTSSY | 401 |
| RIIA or | | CDR-H2: INPVSGST | 402 |
| RIIB | | CDR-H3: ARGGWFDY | 403 |
| | | CDR-L1: SSDVGSYNY | 404 |
| | | CDR-L2: GVS | N/A |
| | | CDR-L3: GTFAGGSYYGV | 405 |

In some embodiments, the binding domain specifically binds to one or more therapeutic targets or antigens in a canine, such as, but are not limited to, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bpi, LFA-1, Neuronal growth factor (NGF), NGFR, NGF-beta, OSMR, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSFi6 (NGFR p75NTR), TNFRSF9 (4-1 BB CD137, ILA), VEFGR-1 (flt-1), VEGF, VEGFR, and VEGFR-3 (flt-4).

In some embodiments, the bispecific binding agent can comprise a protein, wherein the protein is a therapeutic protein, e.g., EPO, CTLA4, LFA3, VEGFR1NEGFR3, IL-1R, IL-4R, GLP-1 receptor agonist, or Thrombopoietin binding peptide. In some embodiments, the therapeutic protein is ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bpi, LFA-1, Neuronal growth factor (NGF), NGFR, NGF-beta, OSMR, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1BB CD137, ILA), VEFGR-1 (flt-1), VEGF, VEGFR, or VEGFR-3 (flt-4).

In some embodiments, the therapeutic protein is any protein described herein. In one embodiment, the bispecific antibody further comprises a modified canine IgG CH₂ domain, IgG CH₃ domain, or IgG Fc domain as described herein. The modified canine IgG CH₂ domain, IgG CH₃ domain, or IgG Fc domain can enhance the half-life the therapeutic proteins in vivo.

### Anti-NGF Antibodies or Antigen-Binding Fragments Thereof

The present disclosure provides an antibody or antigen-binding fragment thereof capable of specifically binding to nerve growth factor (NGF), in which the antibody or antigen-binding fragment thereof includes a complementarity-determining region-heavy chain 1 (CDR-H1), CDR-H2, and CDR-H3, and a complementarity-determining region-heavy chain 1 (CDR-L1), CDR-L2, and CDR-L3, in which: (a) the CDR-H1 includes the amino acid sequence of SEQ ID NO: 55 or a variant thereof with up to two (e.g., one or two) conservative amino acid substitutions; (b) the CDR-H2 includes the amino acid sequence of SEQ ID NO: 56 or a variant thereof with up to two (e.g., one or two) conservative amino acid substitutions; (c) the CDR-H3 includes the amino acid sequence of SEQ ID NO: 57 or a variant thereof with up to two (e.g., one or two) conservative amino acid substitutions; (d) the CDR-L1 includes the amino acid sequence of SEQ ID NO: 58 or a variant thereof with up to two (e.g., one or two) conservative amino acid substitutions; (e) the CDR-L2 includes the amino acid sequence ATS or a variant thereof with up to two (e.g., one or two) conservative amino acid substitutions; and (f) the CDR-L3 includes the amino acid sequence of SEQ ID NO: 59 or a variant thereof with up to two (e.g., one or two) conservative amino acid substitutions.

In some embodiments, (a) the CDR-H1 includes the amino acid sequence of SEQ ID NO: 55; (b) the CDR-H2 includes the amino acid sequence of SEQ ID NO: 56; (c) the CDR-H3 includes the amino acid sequence of SEQ ID NO: 57; (d) the CDR-L1 includes the amino acid sequence of SEQ ID NO: 58; (e) the CDR-L2 includes the amino acid sequence ATS; and (f) the CDR-L3 includes the amino acid sequence of SEQ ID NO: 59. The sequences of these CDR sequences are also provided below:

CDR-H1:
(SEQ ID NO: 55)
GMSLWSNS
CDR-H2:
(SEQ ID NO: 56)
IWSQGGT
CDR-H3:
(SEQ ID NO: 57)
AQIYYYDAEYLHWYFDF
CDR-L1:
(SEQ ID NO: 58)
EGISKN
CDR-L2:
ATS
CDR-L3:
(SEQ ID NO: 59)
QQGYQFPLT

In some embodiments, the antibody or antigen-binding fragment thereof includes one or more of (e.g., one, two, three, four, five, six, seven, or all eight of) a framework region-heavy chain 1 (FR-H1) containing a sequence having at least 80% identity (e.g., 85%, 90%, 95%, 97%, 99%, or 100% identity) to SEQ ID NO: 62, a FR-H2 containing a sequence having at least 80% identity (e.g., 85%, 90%, 95%, 97%, 99%, or 100% identity) to SEQ ID NO: 63, a FR-H3 containing a sequence having at least 80% identity (e.g., 85%, 90%, 95%, 97%, 99%, or 100% identity) to SEQ ID NO: 64, a FR-H4 containing a sequence having at least 80% identity (e.g., 85%, 90%, 95%, 97%, 99%, or 100% identity) to SEQ ID NO: 65, a framework region-light chain 1 (FR-L1) containing a sequence having at least 80% identity (e.g., 85%, 90%, 95%, 97%, 99%, or 100% identity) to SEQ ID NO: 66, a FR-L2 containing a sequence having at least 80% identity (e.g., 85%, 90%, 95%, 97%, 99%, or 100% identity) to SEQ ID NO: 67, a FR-L3 containing a sequence having at least 80% identity (e.g., 85%, 90%, 95%, 97%, 99%, or 100% identity) to SEQ ID NO: 68, and a FR-L4 containing a sequence having at least 80% identity (e.g., 85%, 90%, 95%, 97%, 99%, or 100% identity) to SEQ ID NO: 69.

In some embodiments, the antibody or antigen-binding fragment thereof includes one or more of (e.g., one, two, three, four, five, six, seven, or all eight of) a FR-H1 containing the sequence of SEQ ID NO: 62, a FR-H2 containing the sequence of SEQ ID NO: 63, a FR-H3 containing the sequence of SEQ ID NO: 64, a FR-H4 containing the sequence of SEQ ID NO: 65, a FR-L1 containing the sequence of SEQ ID NO: 66, a FR-L2 containing the sequence of SEQ ID NO: 67, a FR-L3 containing the sequence of SEQ ID NO: 68, and a FR-L4 containing the sequence of SEQ ID NO: 69. In some embodiments, the antibody or antigen-binding fragment thereof includes a FR-H1 containing the sequence of SEQ ID NO: 62, a FR-H2 containing the sequence of SEQ ID NO: 63, a FR-H3 containing the sequence of SEQ ID NO: 64, a FR-H4 containing the sequence of SEQ ID NO: 65, a FR-L1 containing the sequence of SEQ ID NO: 66, a FR-L2 containing the sequence of SEQ ID NO: 67, a FR-L3 containing the sequence of SEQ ID NO: 68, and a FR-L4 containing the sequence of SEQ ID NO: 69. These framework region sequences are also provided below:

FR-H1:
(SEQ ID NO: 62)
EVQLVESGGDLVAPSQSLSITCTVSQQGYQFPLT
FR-H2:
(SEQ ID NO: 63)
ISWVRQPPGRGLEWLGT
FR-H3:
(SEQ ID NO: 64)
TETDAVKGRFTISRDNAKNTVYLQMNSLRAEDTAMYYC
FR-H4:
(SEQ ID NO: 65)
WGQGTLVTVSS
FR-L1:
(SEQ ID NO: 66)
EIVMTQSPASLSASQEEKVTITCRAS
FR-L2:
(SEQ ID NO: 67)
LAWYQQKPGQAPKLLIH
FR-L3:
(SEQ ID NO: 68)
HLQTGVPSRFSGSGSGTDFTLTISSLQPEODFATYYC
FR-L4:
(SEQ ID NO: 69)
FGQGTKVEIK

In some embodiments, the antibody or antigen-binding fragment thereof includes a heavy chain variable region (VH) containing a sequence having at least 80% identity (e.g., 85%, 90%, 95%, 97%, 99%, or 100% identity) to SEQ ID NO: 60, and a light chain variable region (VL) containing a sequence having at least 80% identity (e.g., 85%, 90%, 95%, 97%, 99%, or 100% identity) to SEQ ID NO: 61.

In some embodiments, the VH includes the sequence of SEQ ID NO: 60 and the VL includes the sequence of SEQ ID NO: 61. These variable region sequences are also provided below:

VH:
(SEQ ID NO: 60)
EVQLVESGGDLVAPSQSLSITCTVSGMSLWSNSISWVRQPPGRGL
EWLGTIWSQGGTTETDAVKGRFTISRDNAKNTVYLQMNSLRAEDT
AMYYCAQIYYYDAEYLHWYFDFWGQGTLVTVSS
VL:
(SEQ ID NO: 61)
EIVMTQSPASLSASQEEKVTITCRASEGISKNLAWYQQKPGQAPK
LLIHATSHLQTGVPSRFSGSGSGTDFTLTISSLQPEDFATYYCQQ
GYQFPLTFGQGTKVEIK

In some embodiments, the antibody or antigen-binding fragment thereof is a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, an antibody-like protein scaffold, a Fv fragment, a Fab fragment, a F(ab′)2 molecule, or a tandem scFv (taFv).

In some embodiments, the antibody or antigen-binding fragment thereof is of IgG, IgA, IgM, IgD, or IgE isotype. In some embodiments, the antibody or antigen-binding fragment thereof is of IgG isotype. In some embodiments, the antibody or antigen-binding fragment thereof is of canine IgGA, IgGB, IgGC, or IgGD subclass. In some embodiments, the antibody or antigen-binding fragment thereof is of canine IgGB subclass.

In some embodiments, the antibody or antigen-binding fragment thereof includes a canine Fc domain variant containing at least one of (e.g., one, two, three, four, five, six, seven, or all eight of) the following amino acid substitutions: (a) 252Y and, optionally, at least one amino acid substitution selected from the group consisting of 251 D or 251 E; 285N or 285D; 286D, 286Y, 286F, 286L, or 286W; 307Q; 308P; 315D; 426L, 426H, 426F, or 426Y; 430A or 430K; 433K; 435Y; and 436H; (b) 252M and, optionally, at least one amino acid substitution selected from the group consisting of 251 D or 251 E; 256D or 256F; 285N or 285D; 286D, 286Y, 286F, 286L, or 286W; 307Q; 308P; 315D; 426L, 426H, 426F, or 426Y; 430A or 430K; 433K; 435Y; and 436H; (c) 434H and, optionally, at least one amino acid substitution selected from the group consisting of 286Y, 286F, 286L, or 286W; and 426Y, 426F, 426L, or 426W; (d) 434R; (e) 426Y and, optionally, at least one amino acid substitution selected from the group consisting of 286F, 286W, 286L, or 286Y; 312P; 434R; and 436H; (f) 426H and, optionally, at least one amino acid substitution selected from the group consisting of 286F, 286W, 286L, or 286Y; 312P; 434R; and 436H; (g) 426F and, optionally, at least one amino acid substitution selected from the group consisting of 286F, 286W, 286L, or 286Y; 312P; 434R; and 426H; or (h) 434R and, optionally, at least one amino acid substitution selected from the group consisting of 286L; 286Y; 312P; and 436H, in which the amino acid positions are based on EU numbering. In some embodiments, the canine Fc domain variant includes the amino acid substitutions 426Y and 286L.

In some embodiments, the canine Fc domain variant further includes the amino acid substitutions 234A and 235A, in which the amino acid positions are based on EU numbering.

In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NOs: 41-44. In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NO: 42. In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NO: 41. In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NO: 43. In some embodiments, the antibody or antigen-binding fragment thereof includes a canine Fc domain variant containing a sequence having at least 80% identity (e.g., 85%, 90%, 95%, 97%, 99%, or 100% identity) to any one of SEQ ID NO: 44.

In some embodiments, the antibody or antigen-binding fragment thereof includes a heavy chain containing a sequence having at least 80% identity (e.g., 85%, 90%, 95%, 97%, 99%, or 100% identity) to SEQ ID NO: 26 and a light chain containing a sequence having at least 80% identity (e.g., 85%, 90%, 95%, 97%, 99%, or 100% identity) to SEQ ID NO: 27.

In some embodiments, the heavy chain includes the sequence of SEQ ID NO: 26 and the light chain includes the sequence of SEQ ID NO: 27. These heavy and light chain sequences are also provided below:

Heavy chain:
(SEQ ID NO: 26)
EVQLVESGGDLVAPSQSLSITCTVSGMSLWSNSISWVRQPPGRGL
EWLGTIWSQGGTTETDAVKGRFTISRDNAKNTVYLQMNSLRAEDT
AMYYCAQIYYYDAEYLHWYFDFWGQGTLVTVSSASTTAPSVFPLA
PSCGSTSGSTVALACLVSGYFPEPVTVSWNSGSLTSGVHTFPSVL
QSSGLYSLSSMVTVPSSRWPSETFTCNVAHPASKTKVDKPVPKRE
NGRVPRPPDCPKCPAPEAAGGPSVFIFPPKPKDTLLIARTPEVTC
VVVDLDPEDPEVQISWFVDGKQMQLAKTQPREEQFNGTYRVVSVL
PIGHQDWLKGKQFTCKVNNKALPSPIERTISKARGQAHQPSVYVL
PPCREELSKNTVSLWCLIKDFFPPDIDVEWQSNGQQEPESKYRTT
PPQLDEDGSYFLYSKLSVDKSRWQRGDTFICYVMHEALHNHYTQE
SLSHSPGK
Light chain:
(SEQ ID NO: 27)
EIVMTQSPASLSASQEEKVTITCRASEGISKNLAWYQQKPGQAPK
LLIHATSHLQTGVPSRFSGSGSGTDFTLTISSLQPEDFATYYCQQ
GYQFPLTFGQGTKVEIKRNDAQPAVYLFQPSPDQLHTGSASVVCL
LNSFYPKDINVKWKVDGVIQDTGIQESVTEQDKDSTYSLSSTLTM
SSTEYLSHELYSCEITHKSLPSTLIKSFQRSECQRVD

Other Substitutions that can be Included in the Antibodies

The development of a therapeutic polypeptide or protein (e.g., a bispecific antibody, an antibody, or an antigen-binding fragment thereof) is a complex process that entails coordination of a complex set of activities to generate the desired polypeptide or protein. These include optimization of the specificity, affinity, functional activity, expression level in engineered cell lines, long-term stability, elimination or enhancement of effector functions and development of commercially viable manufacturing and purification methods. This disclosure encompasses substitutions at one or more additional amino acid positions within the Fc domain (or one or both of the Fc subunits) of the antibodies that facilitate any one or more of the above goals.

In some embodiments, the Fc domain (or one or both of the Fc subunits) described herein includes amino acid substitutions at one or more additional amino acid positions that increase or decrease effector function and/or improve product heterogeneity.

In some embodiments, the substitutions are introduced to reduce effector function of the Fc domain (e.g., the Fc domain formed by a first Fc subunit and a second Fc subunit). Such substitutions may be at one or more (e.g., 1, 2, 3, 4, 5, 6, or 7) of the following positions of the canine IgG (numbering according to EU numbering): 238, 265, 297, 298, 299, 327, and 329. The substitution(s) can be to any of the other 19 amino acids. In some embodiments, the substitution is conservative. In certain non-limiting embodiments, the substituted amino acid at position 238 is Ala; the substituted amino acid at position 265 is Ala; the substituted amino acid at position 297 is Ala or Gln; the substituted amino acid at position 298 is Pro; the substituted amino acid at position 299 is Ala; the substituted amino acid at position 327 is Gly; and the substituted amino acid at position 329 is Ala. In some embodiments, the Fc subunit is from, or is derived from, a canine IgGB or IgGC antibody. In some embodiments, the Fc subunit is from, or is derived from, a canine IgGB antibody.

In some embodiments, substitutions are introduced to an Fc domain to enhance its binding to Protein A so as to facilitate purification by protein A chromatography. Such substitutions may be at one or both (e.g., 1, 2, 3, 4, 5, 6, or 7) of the following positions of the canine IgG (numbering according to EU numbering): 252 and 254. The substitution(s) can be to any of the other 19 amino acids. In some embodiments, the substitution is conservative. In certain non-limiting embodiments, the substituted amino acid at position 252 is Met; and the substituted amino acid at position 254 is Ser.

In some embodiments, the Fc domain described herein comprises amino acid substitutions at one or more additional amino acid positions that increase or decrease effector function and/or improve product heterogeneity.

In some embodiments, substitutions are introduced to reduce effector function of the Fc domain. Such substitutions are familiar to a person skilled in the art and may be at one or more (e.g., 1, 2, 3, 4, 5, 6, or 7) positions of the canine IgG. Illustrative examples of such substitutions include those disclosed in WO 2019/035010, which is incorporated herein by reference in its entirety. In some embodiments, the substitution is conservative.

In some embodiments, substitutions are introduced to the Fc domain to enhance its binding to Protein A, thereby facilitating the purification by protein A chromatography. Such substitutions may be at one or more (e.g., 1, 2, 3, 4, 5, 6, or 7) positions of the canine IgG. Illustrative examples of such substitutions include those disclosed in WO 2019/035010.

In some embodiments, substitutions are made to alter the binding affinity of the Fc domain described herein to FcRn as compared to a wild type canine Fc domain (e.g., to increase or decrease binding affinity to FcRn). In some variations, the modification can be one, two, three, or four modifications that are selected from the group consisting of: 308F, 428L, and 434M or 434S, where the numbering is according to the EU numbering. In some embodiments, the Fc subunit includes one or more modifications selected from the group consisting of: 252Y/428L, 428L/434H, 428L/434F, 428L/434Y, 428L/434A, 428L/434M, and 428L/434S, where the numbering is according to the EU numbering. In some embodiments, the Fc variant includes one or more modification selected from the group consisting of: 428L/434S, 308F/428L/434S, where the numbering is according to the EU numbering. In some embodiments, the Fc variant includes one or more modifications selected from the group consisting of: 2591/434S, 308F/434S, 308F/428L/434S, 2591/308F/434S, 307Q/308F/434S, 2501/308F/434S, and 308F/319L/434S, where the numbering is according to the EU numbering. A detailed description of these modifications is described in, e.g., U.S. Pat. No. 8,8839,73, which is incorporated herein by reference in its entirety.

In some embodiments, the bispecific antibody comprises a hinge region of a canine antibody. In some embodiments, modifications can be made to the hinge region of the canine antibody to increase half-life. In some embodiments, the modification is 228P according to EU numbering.

In some embodiments, the binding of the Fc domain with FcRn is pH-dependent. H310 and H435 (EU numbering) can be critical for pH-dependent binding. Thus, in some embodiments, the amino acids at position 310 (EU numbering) is histidine. In some embodiments, the amino acids at position 435 (EU numbering) is histidine. In some embodiments, the amino acids at both positions are histidine.

In some embodiments, the Fc domain has MALA mutations (M234A and L235A mutations in EU numbering), or MALA-PG mutations (M234A, L235A, P329G mutations in EU numbering). In some embodiments, the Fc domain has a P234A, M234A, S234A, or 1234A mutation. In some embodiments, the amino acid residue at position 234 (EU numbering) is Ala. In some embodiments, the amino acid residue at position 234 (EU numbering) is Ala. In some embodiments, the amino acid residues at positions 234 and 235 (EU numbering) are Ala. In some embodiments, the amino acid residues at position 329 (EU numbering) is Gly.

### Pharmaceutical Compositions

The present disclosure provides a pharmaceutical composition that includes any bispecific antibody of the disclosure and a pharmaceutically acceptable excipient. The present disclosure also provides a pharmaceutical composition that includes any antibody or antigen-binding fragment thereof of the disclosure and a pharmaceutically acceptable excipient.

To prepare pharmaceutical or sterile compositions of an antibody (e.g., a bispecific antibody, an antibody, or an antigen-binding fragment thereof) described herein, the antibody can be admixed with a pharmaceutically acceptable carrier or excipient. (See, e.g., Remington's Pharmaceutical Sciences and U.S. Pharmacopeia: National Formulary, Mack Publishing Company, Easton, Pa., 1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al., Goodman and Gilman's The Pharmacological Basis of Therapeutics, McGraw-Hill, New York, N.Y., 2022; Gennaro, Remington: The Science and Practice of Pharmacy, Lippincott, Williams, and Wilkins, New York, N.Y., 2006; Avis, et al. (eds.), Pharmaceutical Dosage Forms: Parenteral Medications, Marcel Dekker, N.Y., 2010; Lieberman, et al. (eds.), Pharmaceutical Dosage Forms: Tablets, Marcel Dekker, N.Y., 1996; Lieberman, et al. (eds.), Pharmaceutical Dosage Forms: Disperse Systems, Marcel Dekker, N.Y., 1996; Weiner and Kotkoskie, Excipient Toxicity and Safety, Marcel Dekker, Inc., New York, N.Y., 2007). In one embodiment, the antibody of the present invention is diluted to an appropriate concentration in a sodium acetate solution, pH 5-6, and NaCl or sucrose is added for tonicity. Additional agents, such as polysorbate 20 or polysorbate 80, may be added to enhance stability.

Toxicity and therapeutic efficacy of the compositions (e.g. bispecific antibodies, antibodies or antigen-binding fragments thereof, or pharmaceutical compositions) of the disclosure, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED₅₀ with little or no toxicity. The dosage may vary within this range depending upon the dosage form employed and the route of administration.

Any suitable mode of administration can be used. Exemplary suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the bispecific antibody can be administered by an invasive route such as by injection. In further embodiments, the bispecific antibody is administered intravenously, subcutaneously, intramuscularly, intraarterially, intratumorally, or by inhalation, aerosol delivery.

Alternatively, one may administer the antibodies of the disclosure in a local rather than systemic manner, for example, via injection of the antibody directly into an arthritic joint or pathogen-induced lesion characterized by immunopathology, often in a depot or sustained release formulation. Furthermore, one may administer the bispecific antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Determination of the appropriate dose of the antibodies of the disclosure is made by one skilled in the art, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

### Nucleic Acids, Vectors, Host Cells, and Methods of Production

The disclosure also encompasses nucleic acid (or nucleic acids) encoding the antibody (e.g., the bispecific antibody, the antibody, or the antigen-binding fragment thereof) described herein, a vector (or vectors) comprising the nucleic acid (or nucleic acids), and host cells comprising the nucleic acid (or nucleic acids) or the vector (or vectors).

In one aspect, the disclosure features a nucleic acid encoding any bispecific antibody of the disclosure or any antibody or antigen-binding fragment thereof of the disclosure. The nucleic acid may be an isolated or synthetic nucleic acid.

In one aspect, the disclosure features a vector including any nucleic acid of the disclosure. In some embodiments, the vector is an expression vector. The vector may be an isolated or synthetic vector.

In one aspect, the disclosure features an isolated host cell including any nucleic acid of the disclosure or any vector of the disclosure. The host cell may be an isolated host cell.

In one aspect, the disclosure features a method of making any bispecific antibody of the disclosure, or any antibody or antigen-binding fragment thereof of the disclosure, the method including: (a) providing the corresponding nucleic acid or the vector; and (b) expressing the corresponding nucleic acid or the vector in a host cell culture, thereby producing the bispecific antibody, or the antibody or antigen-binding fragment thereof of the disclosure.

In some embodiments, the method further includes (c) collecting the bispecific antibody or the antibody or antigen-binding fragment thereof produced in (b) from the host cell culture.

The antibody (e.g., the bispecific antibody, the antibody or antigen-binding fragment thereof) described herein may be produced in bacterial or eukaryotic cells. Some or all polypeptide constituents of the antibody, e.g., Fabs, heavy chains, or light chains, can be produced in bacterial cells, e.g., E. coli cells. Polypeptide constituents of the antibody of the disclosure can also be produced in eukaryotic cells, such as transformed cell lines (e.g., Chinese hamster ovary (CHO) cells, 293E cells, COS cells, 293T cells, and HeLa cells). In addition, polypeptide constituents of the antibody of the disclosure can be expressed in a yeast cell, such as Pichia (see, e.g., Powers et al., J Immunol Methods. 251: 123-35, 2001), Hanseula, or Saccharomyces. To produce any antibody of interest, a nucleic acid (or nucleic acids) encoding the antibody is/are constructed, introduced into a vector (or vectors) (e.g., an expression vector or expression vectors), and then expressed in suitable host cells. To improve expression, the nucleotide sequences of the genes encoding one or more polypeptide constituents of the antibody can be recoded without changing (or with minimally changing, e.g., removal of a C-terminal residue of the heavy or light chain) the amino acid sequence of the antibody. The areas for potential recoding include those associated with translation initiation, codon usage, and possible unintended mRNA splicing. Nucleic acids encoding an Fc domain or an Fc subunit described herein would be readily envisioned by the ordinarily skilled artisan, e.g., based on the knowledge of the sequence of a wild type Fc domain and any modifications (e.g., amino acid substitutions, deletions, additions, or modifications) made to the Fc domain.

Standard molecular biology techniques can be used to prepare the vector(s) (e.g., the expression vectors or recombinant expression vectors), transfect the host cells, select for transformants, culture the host cells, and recover the antibody (e.g., e.g., the bispecific antibody, the antibody or antigen-binding fragment thereof).

If the antibody is to be expressed in bacterial cells (e.g., E. coli), the expression vector may have characteristics that permit amplification of the vector in the bacterial cells. Additionally, when E. coli such as JM109, DH5α, HB101, or XL1-Blue are used as hosts, the vector may have a promoter, for example, a lacZ promoter (Ward et al., 341: 544-546, 1989), araB promoter (Better et al., Science, 240: 1041-1043, 1988), or T7 promoter that can allow efficient expression in E. coli. Examples of such vectors include, for example, M13-series vectors, pUC-series vectors, pBR322, PBLUESCRIPT®, pCR-Script, pGEX-5X-1 (Pharmacia), QIAEXPRESS® system (QIAGEN), pEGFP, and pET (when this expression vector is used, the host is preferably a BL21 cell expressing T7 RNA polymerase). The expression vector may contain a signal sequence for antibody secretion. For production into the periplasm of E. coli, the pe/B signal sequence (Lei et al., J. Bacteriol., 169: 4379, 1987) may be used as the signal sequence for antibody secretion. For bacterial expression of the antibody, calcium chloride methods or electroporation methods may be used to introduce the expression vector into the bacterial cell.

If the antibody is to be expressed in animal cells such as CHO, COS, and NIH3T3 cells, the expression vector may include a promoter for expression in these cells, for example, an SV40 promoter (Mulligan et al., Nature, 277: 108, 1979) (e.g., early simian virus 40 promoter), MMLV-LTR promoter, EF1α promoter (Mizushima et al., Nucleic Acids Res., 18: 5322, 1990), or CMV promoter (e.g., human cytomegalovirus immediate early promoter). In addition to the nucleic acid sequence encoding the Fc domain variant, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017). For example, typically the selectable marker gene confers resistance to drugs, such as G418, hygromycin, or methotrexate, on a host cell into which the vector has been introduced. Vectors with selectable markers include pMAM, pDR2, pBK-RSV, pBK-CMV, pOPRSV, and pOP13.

In some embodiments, the antibody is produced in mammalian cells. Mammalian host cells for expressing polypeptide or polypeptides (e.g., bispecific antibody) include Chinese hamster ovary (CHO cells), human embryonic kidney (HEK) 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal.

In an exemplary system for antibody expression, a recombinant expression vector (or recombinant expression vectors) encoding the one or more antibody heavy chains and the one or more antibody light chains of the antibody is introduced into dhfr-CHO cells by calcium phosphate-mediated transfection. Within the recombinant expression vector, the antibody heavy chain genes and/or the antibody light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection or amplification. The selected transformant host cells are cultured to allow for the expression of the antibody heavy and light chains, and the antibody is recovered from the culture medium of the host cells.

### Methods of Treatment

The antibody disclosed herein can be used to treat or prevent any disease or disorder in a canine in need thereof.

In one aspect, the disclosure provides a method of treating or preventing a canine disease or disorder in a dog in need thereof, the method including administering an effective amount of any bispecific antibody of the disclosure, any antibody or antigen-binding fragment thereof of the disclosure, or any pharmaceutical composition of the disclosure.

In some embodiments, the canine disease or disorder is an allergic disease, a chronic pain, an acute pain, a skeletal or musculoskeletal disease, a metabolic disease, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, muscular atrophy, diabetes (e.g., type 1 diabetes or type 2 diabetes), arthritis, anemia, or obesity.

In one aspect, the disclosure provides a method of treating or preventing a pain in a dog in need thereof, the method including administering an effective amount of any antibody or antigen-binding fragment thereof of the disclosure, or a pharmaceutical composition that includes the antibody or antigen-binding fragment thereof, to the dog.

In one aspect, the disclosure provides any bispecific antibody of the disclosure, any antibody or antigen-binding fragment thereof of the disclosure, or any pharmaceutical composition of the disclosure for use in the treatment or prevention of a canine disease or disorder in a dog in need thereof.

In some embodiments, the canine disease or disorder is an allergic disease, a chronic pain, an acute pain, a skeletal or musculoskeletal disease, a metabolic disease, a metabolic disease, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, muscular atrophy, diabetes (e.g., type 1 diabetes or type 2 diabetes), arthritis, anemia, or obesity.

In another aspect, the disclosure provides any antibody or antigen-binding fragment thereof of the disclosure, or a pharmaceutical composition including the antibody or antigen-binding fragment thereof, for use in the treatment or prevention of a pain in a dog in need thereof.

In some embodiments, the disease, disorder, condition, or symptom being treated or prevented is an allergic disease, a chronic pain, an acute pain, an inflammatory disease, an autoimmune disease, an endocrine disease, a gastrointestinal disease, a skeletal or musculoskeletal disease, a metabolic disease, a cardiovascular disease, a neurological disease, a renal disease, a metabolic disease, an immunological disease, a genetic/inherited disease, a fertility related disorder, an infectious disease or a cancer. In certain embodiments, the disease or disorder being treated or prevented is atopic dermatitis, allergic dermatitis, food allergy, osteoarthritic pain, perioperative pain, dental pain, cancer pain, arthritis, anemia, obesity, or diabetes.

Antibodies (e.g., bispecific antibodies, antibodies, or antigen-binding fragments thereof) may not only be used to treat or prevent a disease but also to modulate a normal biological function, for example, to manage fertility or behavior.

A person of ordinary skill in the art understands that the bispecific antibody of the disclosure may be used to treat or prevent a disease, disorder, condition, or symptom in a dog in need of when the bispecific antibody includes at least one (e.g., one or two) antigen-binding moiety that specifically binds to an antigen associated with the disease, disorder, condition, or symptom.

For example, a bispecific antibody that includes at least one antigen-binding moiety that specifically binds to NGF may be used to treat or prevent a pain (e.g., a chronic pain, an acute pain, an osteoarthritic pain, a perioperative pain, a dental pain, or a cancer pain) and/or an inflammatory disease (e.g., atopic dermatitis, rheumatoid arthritis, osteoarthritis, or psoriasis) in a dog in need thereof.

A bispecific antibody that includes at least one antigen-binding moiety that specifically binds to Activin RIIA and/or Activin RIIB may be used to treat or prevent a skeletal or musculoskeletal disease (e.g., sarcopenia, cachexia, muscular atrophy, osteoporosis) and/or a metabolic disease (e.g., obesity, diabetes, or fatty liver disease) in a dog in need thereof. An antigen-binding moiety that binds Activin RIIB may also bind Activin RIIA due to cross-reactivity. Similarly, an antigen-binding moiety that binds Activin RIIA may also bind Activin RIIB due to cross-reactivity.

A bispecific antibody that includes at least one antigen-binding moiety that specifically binds to IL-31 or IL-31R may be used to treat or prevent an allergic disease (e.g., atopic dermatitis, asthma, allergic rhinitis), an inflammatory disease (e.g., psoriasis, atopic dermatitis), and/or an autoimmune disease (e.g., lupus, rheumatoid arthritis) in a dog in need thereof.

In some embodiments, the bispecific antibody includes a first antigen-binding moiety that binds NGF and a second antigen-binding moiety that binds Activin RIIA and/or Activin RIIB. In some embodiments, the bispecific antibody includes a first antigen-binding moiety that binds Activin RIIA and/or Activin RIIB and a second antigen-binding moiety that binds NGF. A bispecific antibody that binds to both NGF and Activin RIIA and/or RIIB may be used to treat or prevent a pain (e.g., a chronic pain, an acute pain, an osteoarthritic pain, a perioperative pain, a dental pain, or a cancer pain), an inflammatory disease (e.g., atopic dermatitis, rheumatoid arthritis, osteoarthritis, or psoriasis), a skeletal or musculoskeletal disease (e.g., sarcopenia, cachexia, muscular atrophy, osteoporosis), and/or a metabolic disease (e.g., obesity, diabetes, or fatty liver disease) in a dog in need thereof. In some embodiments, the bispecific antibody that includes one antigen-binding moiety that binds NGF and another antigen-binding moiety that binds Activin RIIA and/or Activin RIIB of this disclosure may be used to treat or prevent a pain, atopic dermatitis, obesity, and/or diabetes.

A person of ordinary skill in the art also understands that the antibody or antigen-binding fragment that specifically bind to NGF of this disclosure may be used to treat or prevent a pain (e.g., a chronic pain, an acute pain, an osteoarthritic pain, a perioperative pain, a dental pain, or a cancer pain) and/or an inflammatory disease (e.g., rheumatoid arthritis, osteoarthritis, or psoriasis) in a dog in need thereof. In some embodiments, the antibody or antigen-binding fragment that specifically bind to NGF of this disclosure may be used to treat or prevent a pain in a dog in need thereof.

In some embodiments, the antibody (e.g., the bispecific antibody, the antibody or antigen-binding fragment thereof) disclosed herein, or the pharmaceutical composition comprising the antibody disclosed herein, is administered parenterally, by subcutaneous administration, intravenous infusion, or intramuscular injection. In some embodiments, the antibody disclosed herein, or the pharmaceutical composition comprising the antibody disclosed herein, is administered as a bolus injection or by continuous infusion over a period of time. In some embodiments, the antibody disclosed herein, or the pharmaceutical composition comprising the antibody disclosed herein, is administered by an intramuscular, an intraperitoneal, an intracerebrospinal, a subcutaneous, an intra-arterial, an intrasynovial, an intrathecal, or an inhalation route.

In some embodiments, the antibody disclosed herein, or the pharmaceutical composition comprising the antibody disclosed herein, is administered in an amount in the range of 0.01 mg/kg body weight to 50 mg/kg body weight per dose. In some embodiments, the antibody disclosed herein, or the pharmaceutical composition comprising the antibody disclosed herein, is administered 0.01 to 55 mg/kg, 0.01 to 50 mg/kg, 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, monthly, every two months, every three months, every four months, every five months, or every six months, for example. One exemplary dosage of the antibody in canines would be in the range from 0.01 mg/kg to 15 mg/kg. Thus, one or more doses of 0.01 mg/kg, 0.02 mg/kg, 0.04 mg/kg, 0.1 mg/kg, 0.2 mg/kg, 0.4 mg/kg, 1.0 mg/kg, 2.0 mg/kg, 4.0 mg/kg, 10 mg/kg, or 15 mg/kg (or any combination thereof) may be administered to the animal. In some embodiments, the antibody disclosed herein, or the pharmaceutical composition comprising the antibody disclosed herein, is administered 2 mg/kg body weight per dose.

In some embodiments, the antibody (e.g., the bispecific antibody, the antibody or antigen-binding fragment thereof) disclosed herein, or the pharmaceutical composition comprising the antibody disclosed herein, is administered within one, two, three, four, five, or six months, or within one, two, or three weeks, of each other. In some embodiments, the antibody disclosed herein, or the pharmaceutical composition comprising the antibody disclosed herein, is administered every week. In some embodiments, the antibody disclosed herein, or the pharmaceutical composition comprising the antibody disclosed herein, is administered every two weeks. In some embodiments, the antibody disclosed herein, or the pharmaceutical composition comprising the antibody disclosed herein, is administered every three weeks. In some embodiments, the antibody disclosed herein, or the pharmaceutical composition comprising the antibody disclosed herein, is administered every month. In some embodiments, the antibody disclosed herein, or the pharmaceutical composition comprising the antibody disclosed herein, is administered every two months. In some embodiments, the antibody disclosed herein, or the pharmaceutical composition comprising the antibody disclosed herein, is administered every three months. In some embodiments, the antibody disclosed herein, or the pharmaceutical composition comprising the antibody disclosed herein, is administered every four months. In some embodiments, the antibody disclosed herein, or the pharmaceutical composition comprising the antibody disclosed herein, is administered every five months. In some embodiments, the antibody disclosed herein, or the pharmaceutical composition comprising the antibody disclosed herein, is administered every six months. In some embodiments, the antibody disclosed herein, or the pharmaceutical composition comprising the antibody disclosed herein, is administered to a dog at one time or over a series of treatments. In some embodiments, the antibody is administered once per week for at least two or three consecutive weeks, and in some embodiments, this cycle of treatment is repeated two or more times, optionally interspersed with one or more weeks of no treatment.

In some embodiments, the antibody (e.g., the bispecific antibody, the antibody or antigen-binding fragment thereof) disclosed herein, or the pharmaceutical composition comprising the antibody disclosed herein, is administered in combination, concurrently, sequentially, or in conjunction with one or more further therapeutic agents. In some embodiments, the antibody disclosed herein, or the pharmaceutical composition comprising the antibody disclosed herein, is administered in combination, with one or more further therapeutic agents. In some embodiments, the antibody disclosed herein, or the pharmaceutical composition comprising the antibody disclosed herein, is administered concurrently with one or more further therapeutic agents. In some embodiments, the antibody disclosed herein, or the pharmaceutical composition comprising the antibody disclosed herein, is administered sequentially with one or more further therapeutic agents. In some embodiments, the antibody disclosed herein, or the pharmaceutical composition comprising the antibody disclosed herein, is administered in conjunction with one or more further therapeutic agents. Any suitable further therapeutic agents may be used.

### Diagnosis

The antibody (e.g., the bispecific antibody, the antibody or antigen-binding fragment thereof) disclosed herein can also be used for various diagnostic purposes, for example, to determine whether a dog has any particular disease or disorder. In some embodiments, the antibody may comprise a binding domain. The binding domain can specifically bind to a protein, subunit, domain, motif, and/or epitope as described herein (e.g., an antigen). In some embodiments, the antibody further includes a labeling group. In general, labeling groups fall into a variety of classes, depending on the assay in which they are to be detected. Exemplary labeling groups include: (a) isotopic labels, which may be radioactive or heavy isotopes; (b) magnetic labels (e.g., magnetic particles); (c) redox active moieties; (d) optical dyes; (e) enzymatic groups (e.g., horseradish peroxidase, β-galactosidase, luciferase, alkaline phosphatase); (f) biotinylated groups; and (g) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antibody via spacer arms of various lengths to reduce potential steric hindrance. Various methods of labelling proteins (e.g., antibodies) are known in the art and may be used in performing the present invention. In some embodiments, the labeling group is a probe, a dye (e.g., a fluorescent dye), or a radioactive isotope (e.g., ³H, ¹⁴C, ²²Na, ³⁶Cl, ³⁵S, ³³P, or ¹²⁵I).

Specific labels can also include optical dyes, including, but not limited to, chromophores, phosphors, and fluorophores, with the latter being specific in many embodiments. Fluorophores can be either “small molecule” fluores, or proteinaceous fluores.

The fluorescent label can be any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malachite green, stilbene, Lucifer Yellow, CASCADE® Blue, IAEDANS, EDANS, BODIPY FL, LC Red 640, LC® Red 705, OREGON GREEN®, the ALEXA FLUOR®dyes (ALEXA FLUOR®350, ALEXA FLUOR®430, ALEXA FLUOR®488, ALEXA FLUOR®546, ALEXA FLUOR®568, ALEXA FLUOR®594, ALEXA FLUOR®633, ALEXA FLUOR®660, ALEXA FLUOR®680), CASCADE® Yellow, and R-phycoerythrin (PE) (Molecular Probes, Eugene, Oreg.), FITC, rhodamine, and TEXAS RED®(Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, which is incorporated by reference in its entirety.

Suitable proteinaceous fluorescent labels also include, but are not limited to, green fluorescent protein (GFP), including a Renilla, Ptilosarcus, or Aequorea species of GFP (Chalfie et al., Science 263: 802-805, 1994), Enhanced GFP (EGFP) (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H1J9; Stauber, Biotechniques 24: 462-471, 1998; Heim et al., Curr. Biol. 6: 178-182, 1996), enhanced yellow fluorescent protein (EYFP, Clontech Laboratories, Inc.), luciferase (Ichiki et al., J. Immunol. 150: 5408-5417, 1993), β galactosidase (Nolan et al., Proc. Natl. Acad. Sci. USA. 85: 2603-2607, 1998) and Renilla (International Pat. Application Publication Nos. WO 92/15673, WO 95/07463, WO 98/14605, WO 98/26277, and WO 99/49019; and U.S. Pat. Nos. 5,292,658; 5,418,155; 5,683,888; 5,741,668; 5,777,079; 5,804,387; 5,874,304; 5,876,995; and 5,925,558). All of the above-cited references in this paragraph are expressly incorporated herein by reference in their entirety.

### Assays

### Fc_{γ}RI and FcγRIII Binding

Binding to FcγRI and FcγRlll is a measure of the ability of an antibody to mediate antibody-dependent cell-mediated cytotoxicity (ADCC). In order to assess this property for an antibody an assay to measure binding of the antibody to FcγRI and FcγRlll can be conducted using methods known in the art.

### C1q Binding

Binding to the first component of the complement system, C1q, is a measure of the ability of an antibody to mediate complement-dependent cytotoxicity (CDC). In order to assess this property for an antibody, an assay to measure the binding of the antibody to C1q can be conducted using methods known in the art.

### Half-Life

Methods of measuring half-life of an antibody are well known in the art. See, e.g., Booth et al., MAbs, 10(7): 1098-1110, 2018. As an example, the half-life of an antibody (e.g., a bispecific antibody, an antibody, or an antigen-binding fragment thereof) can be measured by injection of the antibody into an animal model (e.g., a dog model) and measuring the levels of the antibody in the serum over a certain period of time. Other exemplary animal models include non-human primate models and transgenic mouse models. The transgenic mouse models can be null for mouse FcRn alpha chain and express the canine FcRn alpha transgene (e.g., under the control of a constitutive promoter). The canine FcRn alpha chain can pair in vivo with the mouse β2-microglobulin protein forming a functional chimeric FcRn heterodimer. As an example, the half-life of a canine antibody can be measured by injection of the antibody into a dog model and measuring levels of the antibody in the serum over a certain period of time.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Initial Evaluation of CH1-CL CrossMab in Canine IgGB and Kappa or Lambda Constant Regions

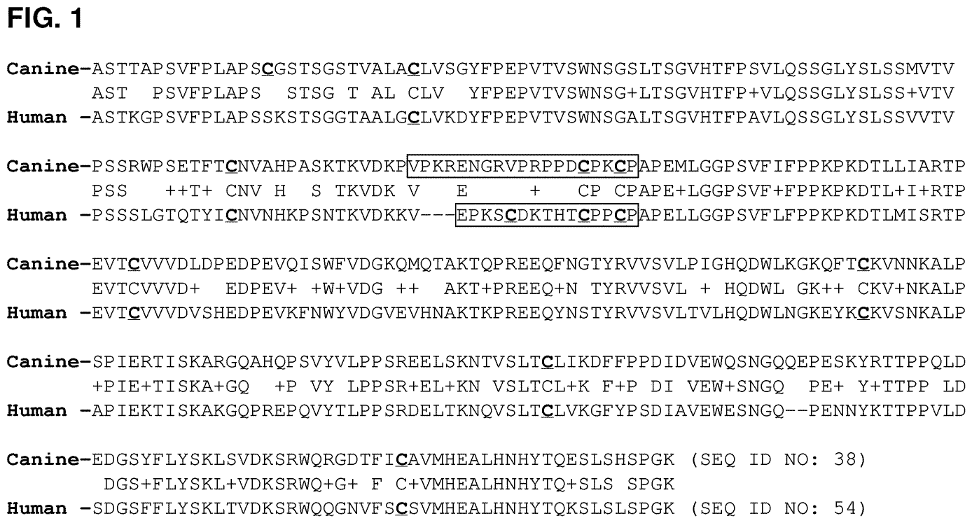

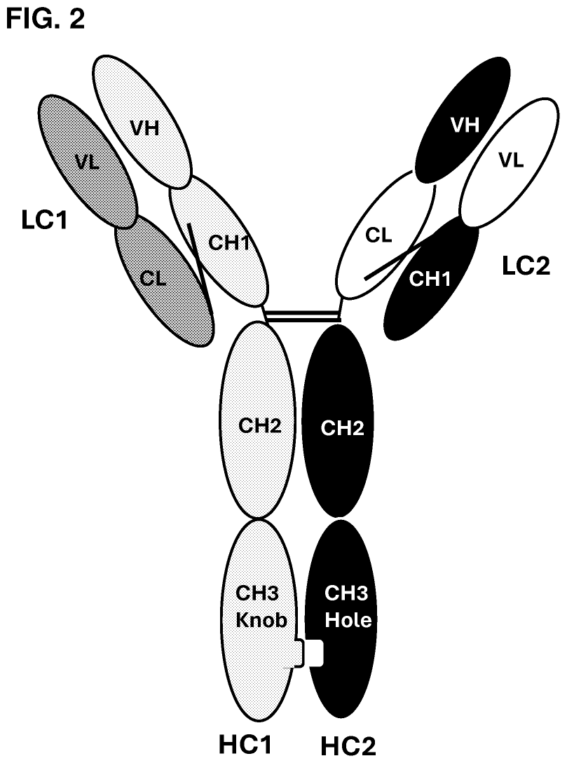

To evaluate whether CH1-CL CrossMab technology (as described in, e.g., International Patent Application Publication Nos. WO 2015/150447 and WO 2017/055539, which are herein incorporated by reference) would work for canine IgGs, the sequence similarity between the human and canine constant regions was assessed. Unlike human IgG1, the canine IgGB (also known as canine IGHG2) has a cysteine early in the CH1 constant region and only has two cysteines in the hinge region. Human IgG1 has three cysteines in the hinge region, the first of which pairs to the light chain constant domain (see FIG. 1; alignment of canine and human IgG). The last cysteine in the canine CH1 is not thought to pair with the light chain for an interchain disulfide bond, but is part of the intrachain CH1 disulfide bond, like the human residue that it aligns with (Frangione and Milstein, Nature 216: 939-941, 1967). It was unclear what additional sequence after the last CH1 cysteine would be necessary to keep the stability of the canine CH1 domain. Therefore, the crossed light chain was designed with variable lengths of the CH1 domain to experimentally determine how much sequence was required. A general schematic of the canine design, termed herein CanBiMab, is represented in FIG. 2.

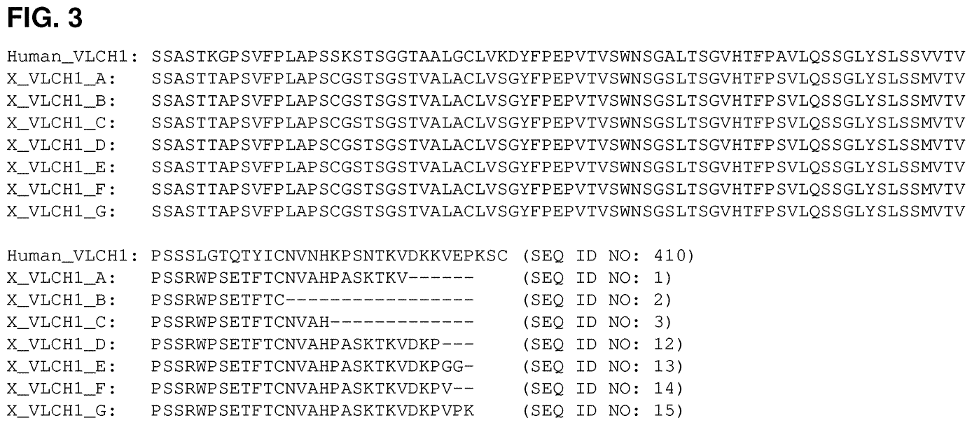

The first set of constructs designed included a serine-serine linker after the variable light (kappa or lambda, see Table 3 below) followed by the CH1 domain with 11 additional residues after cysteine 197, one that ended right after the cysteine (SEQ ID NO: 2), and a third that only kept four amino acids after the cysteine and stopped before proline 202 which often changes the direction of a protein chain (SEQ ID NO: 3). FIG. 3 shows an alignment of these constructs.

TABLE 3
Sequences of initial CanBiMab constant region designs

| SEQ ID | | |
|---|---|---|
| NO: | Chain | Sequence |
| 1 | Constant Canine | SSASTTAPSVFPLAPSCGSTSGSTVALACLVSGYFPEPVTVSWNSGSLTSGVHTFP |
| | X_VLCH1_A | SVLQSSGLYSLSSMVTVPSSRWPSETFTCNVAHPASKTKV |
| 2 | Constant Canine | SSASTTAPSVFPLAPSCGSTSGSTVALACLVSGYFPEPVTVSWNSGSLTSGVHTFP |
| | X_VLCH1_B | SVLQSSGLYSLSSMVTVPSSRWPSETFTC |
| 3 | Constant Canine | SSASTTAPSVFPLAPSCGSTSGSTVALACLVSGYFPEPVTVSWNSGSLTSGVHTFP |
| | X_VLCH1_C | SVLQSSGLYSLSSMVTVPSSRWPSETFTCNVAH |
| 4 | Constant Canine | ASDAQPAVYLFQPSPDQLHTGSASVVCLLNSFYPKDINVKWKVDGVIQDTGIQESV |
| | X_VHkappa_Hole_Z | TEQDKDSTYSLSSTLTMSSTEYLSHELYSCEITHKSLPSTLIKSFQRSECQRVDNVA |
| | | HPASKTKVDKPVPKRENGRVPRPPDCPKCPAPEAAGGPSVFIFPPKPKDTLLIART |
| | | PEVTCVVVDLDPEDPEVQISWFVDGKQMQLAKTQPREEQFNGTYRVVSVLPIGHQ |
| | | DWLKGKQFTCKVNNKALPSPIERTISKARGQAHQPSVCVLPPSREELSKNTVSLSC |
| | | AIKDFFPPDIDVEWQSNGQQEPESKYRTTPPQLDEDGSYFLVSKLSVDKSRWQRG |
| | | DTFICYVMHEALHNRFTQESLSHSPGK |
| 5 | Constant Canine | ASDAQPAVYLFQPSPDQLHTGSASVVCLLNSFYPKDINVKWKVDGVIQDTGIQESV |
| | X_VHkappa_Hole_Y | TEQDKDSTYSLSSTLTMSSTEYLSHELYSCEITHKSLPSTLIKSFQRSECVPKRENG |
| | | RVPRPPDCPKCPAPEAAGGPSVFIFPPKPKDTLLIARTPEVTCVVVDLDPEDPEVQI |
| | | SWFVDGKQMQLAKTQPREEQFNGTYRVVSVLPIGHQDWLKGKQFTCKVNNKALP |
| | | SPIERTISKARGQAHQPSVCVLPPSREELSKNTVSLSCAIKDFFPPDIDVEWQSNGQ |
| | | QEPESKYRTTPPQLDEDGSYFLVSKLSVDKSRWQRGDTFICYVMHEALHNRFTQE |
| | | SLSHSPGK |
| 6 | Constant Canine | ASDAQPAVYLFQPSPDQLHTGSASVVCLLNSFYPKDINVKWKVDGVIQDTGIQESV |
| | X_VHkappa_Hole_X | TEQDKDSTYSLSSTLTMSSTEYLSHELYSCEITHKSLPSTLIKSFQRSECQRVDPAS |
| | | KTKVDKPVPKRENGRVPRPPDCPKCPAPEAAGGPSVFIFPPKPKDTLLIARTPEVT |
| | | CVVVDLDPEDPEVQISWFVDGKQMQLAKTQPREEQFNGTYRVVSVLPIGHQDWL |
| | | KGKQFTCKVNNKALPSPIERTISKARGQAHQPSVCVLPPSREELSKNTVSLSCAIKD |
| | | FFPPDIDVEWQSNGQQEPESKYRTTPPQLDEDGSYFLVSKLSVDKSRWQRGDTFI |
| | | CYVMHEALHNRFTQESLSHSPGK |
| 7 | Constant Canine | ASDAQPAVYLFQPSPDQLHTGSASVVCLLNSFYPKDINVKWKVDGVIQDTGIQESV |
| | X_VHkappa_Hole_W | TEQDKDSTYSLSSTLTMSSTEYLSHELYSCEITHKSLPSTLIKSFQRSECNVAHPAS |
| | | KTKVDKPVPKRENGRVPRPPDCPKCPAPEAAGGPSVFIFPPKPKDTLLIARTPEVT |
| | | CVVVDLDPEDPEVQISWFVDGKQMQLAKTQPREEQFNGTYRVVSVLPIGHQDWL |
| | | KGKQFTCKVNNKALPSPIERTISKARGQAHQPSVCVLPPSREELSKNTVSLSCAIKD |
| | | FFPPDIDVEWQSNGQQEPESKYRTTPPQLDEDGSYFLVSKLSVDKSRWQRGDTFI |
| | | CYVMHEALHNRFTQESLSHSPGK |
| 8 | Constant Canine | ASDAQPAVYLFQPSPDQLHTGSASVVCLLNSFYPKDINVKWKVDGVIQDTGIQESV |
| | X_VHkappa_Hole_V | TEQDKDSTYSLSSTLTMSSTEYLSHELYSCEITHKSLPSTLIKSFQRSECQRVDVPK |
| | | RENGRVPRPPDCPKCPAPEAAGGPSVFIFPPKPKDTLLIARTPEVTCVVVDLDPED |
| | | PEVQISWFVDGKQMQLAKTQPREEQFNGTYRVVSVLPIGHQDWLKGKQFTCKVN |
| | | NKALPSPIERTISKARGQAHQPSVCVLPPSREELSKNTVSLSCAIKDFFPPDIDVEW |
| | | QSNGQQEPESKYRTTPPQLDEDGSYFLVSKLSVDKSRWQRGDTFICYVMHEALH |
| | | NRFTQESLSHSPGK |
| 9 | Constant Canine | ASKASPSVTLFPPSSEELGANKATLVCLISDFYPSGVTVAWKADGSPVTQGVETTK |
| | X_VHlambda_Hole_Z | PSKQSNNKYAASSYLSLTPDKWKSHSSFSCLVTHEGSTVEKKVAPAECSNVAHPA |
| | | SKTKVDKPVPKRENGRVPRPPDCPKCPAPEAAGGPSVFIFPPKPKDTLLIARTPEV |
| | | TCVVVDLDPEDPEVQISWFVDGKQMQLAKTQPREEQFNGTYRVVSVLPIGHQDWL |
| | | KGKQFTCKVNNKALPSPIERTISKARGQAHQPSVCVLPPSREELSKNTVSLSCAIKD |
| | | FFPPDIDVEWQSNGQQEPESKYRTTPPQLDEDGSYFLVSKLSVDKSRWQRGDTFI |
| | | CYVMHEALHNRFTQESLSHSPGK |
| 10 | Constant Canine | ASKASPSVTLFPPSSEELGANKATLVCLISDFYPSGVTVAWKADGSPVTQGVETTK |
| | X_VHlambda_Hole_Y | PSKQSNNKYAASSYLSLTPDKWKSHSSFSCLVTHEGSTVEKKVAPAECVPKRENG |
| | | RVPRPPDCPKCPAPEAAGGPSVFIFPPKPKDTLLIARTPEVTCVVVDLDPEDPEVQI |
| | | SWFVDGKQMQLAKTQPREEQFNGTYRVVSVLPIGHQDWLKGKQFTCKVNNKALP |
| | | SPIERTISKARGQAHQPSVCVLPPSREELSKNTVSLSCAIKDFFPPDIDVEWQSNGQ |
| | | QEPESKYRTTPPQLDEDGSYFLVSKLSVDKSRWQRGDTFICYVMHEALHNRFTQE |
| | | SLSHSPGK |
| 11 | Constant Canine | ASKASPSVTLFPPSSEELGANKATLVCLISDFYPSGVTVAWKADGSPVTQGVETTK |
| | X_VHlambda_Hole_X | PSKQSNNKYAASSYLSLTPDKWKSHSSFSCLVTHEGSTVEKKVAPAECSPASKTK |
| | | VDKPVPKRENGRVPRPPDCPKCPAPEAAGGPSVFIFPPKPKDTLLIARTPEVTCVV |
| | | VDLDPEDPEVQISWFVDGKQMQLAKTQPREEQFNGTYRVVSVLPIGHQDWLKGK |
| | | QFTCKVNNKALPSPIERTISKARGQAHQPSVCVLPPSREELSKNTVSLSCAIKDFFP |
| | | PDIDVEWQSNGQQEPESKYRTTPPQLDEDGSYFLVSKLSVDKSRWQRGDTFICYV |
| | | MHEALHNRFTQESLSHSPGK |

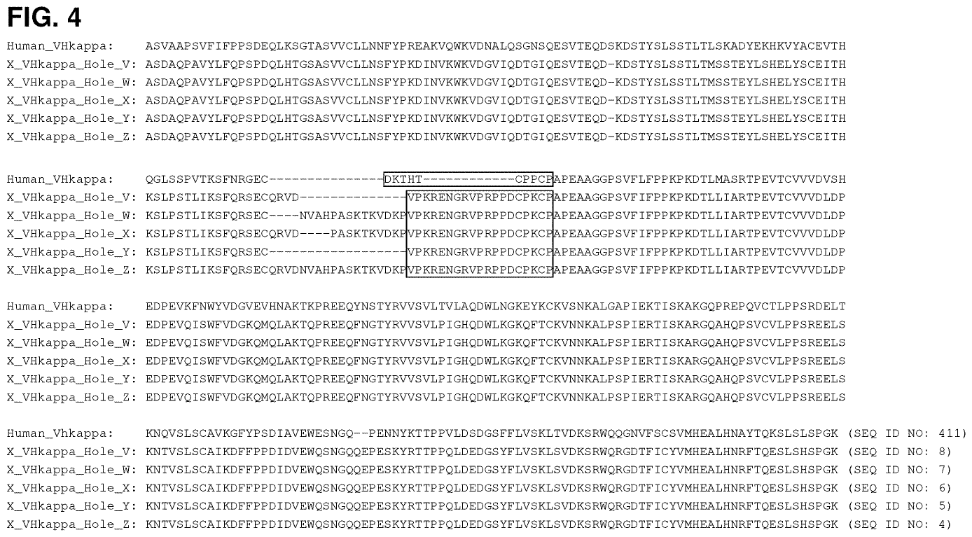

The canine kappa chain has four amino acids after the last cysteine, and it is unclear if these residues are important for stability, especially when being placed in the middle of the heavy constant region. For the VH-kappa heavy chain, it was also unclear how much sequence to keep from the native heavy chain. Five constructs were tested with varying lengths of sequence from these regions (see Table 3 above). Each construct had an alanine serine linker after the variable heavy sequence followed by the kappa region with the first two amino acids removed: with all the remaining kappa sequence and all the sequence after the last CH1 cysteine (Cys 197, SEQ ID NO: 4), removing the four amino acids after the last kappa cysteine and immediately starting at valine 212 (which is part of the hinge region identified by the INTERNATIONAL IMMUNOGENETICS INFORMATION SYSTEM® (IMGT®) as VPKRENGRVPRPPDCPKCP (SEQ ID NO: 5)), keeping all the kappa sequence and starting at proline 202 of the heavy constant region (SEQ ID NO: 6), removing the four amino acids at the end of the kappa chain but keeping all the sequence of the heavy chain after the last CH1 cysteine so starting at asparagine 198 (SEQ ID NO: 7), or keeping all the kappa sequence and then going immediately to the hinge region at valine 212 (SEQ ID NO: 8). Alignments of the sequences are shown in FIG. 4.

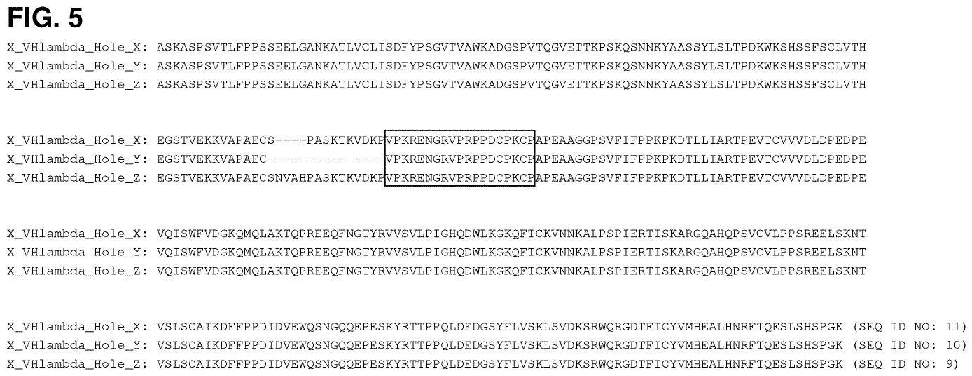

The canine lambda constant region has the addition of a serine after the last cysteine. Three constructs were tested with varying lengths of sequence for the VH-lambda heavy chains (see Table 3 above, and alignments in FIG. 5). Each construct had an alanine serine linker after the variable heavy sequence followed by the lambda constant region with the lambda sequence with the first three amino acids removed; and all the sequence after the cysteine 197 in the CH1 domain (SEQ ID NO: 9), removing the terminal serine of the lambda sequence and then going into the hinge at valine 212 (SEQ ID NO:10), or keeping the serine and starting at proline 202 of the heavy constant region (SEQ ID NO: 11).

To create a heterodimer with the non-crossed chain, canine knob-in-hole mutations previously tested were incorporated into the constant CH2 and CH3 sequence (as described in, e.g., International Patent Application Publication No. WO 2024/155982, which is herein incorporated by reference). On the CanBiMab VH constructs, the “hole” side mutations were made at T366S, L368A, Y407V, and Y349C. These constructs also possessed the half-life extension mutations (A426Y+T286L; as described in, e.g., U.S. Pat. No. 11,434,276, which is herein incorporated by reference) and the MALA mutation (M234A and L235A) to reduce effector function. For purification purposes during the initial validation, the Protein A binding site was knocked-out of the “hole” chain by making H435R and Y436F mutations to allow for preferential purification of the heterodimer (as described in, e.g., U.S. Pat. No. 8,586,713, which is herein incorporated by reference).

The evaluation of CanBiMabs was performed using anti-NGF and anti-Activin RIIB variable regions. The four chains consisted of a CanBiMabs VLCH1, a CanBiMabs VHkappa_Hole (or VHlambda_Hole), a traditional VL-kappa or lambda, and a VH-heavy constant region with knob mutations at T366W and S354C. The sequences of the traditional VL-kappa or lambda and the VH-heavy constant region with knob mutations used in the construction of the initial CanBiMabs are shown in Table 4 below.

TABLE 4
Sequences of additional VL and VH constant regions for initial CanBiMab designs

| SEQ ID | | |
|---|---|---|
| NO: | Chain | Sequence |
| 28 | Traditional Canine VL- | RNDAQPAVYLFQPSPDQLHTGSASVVCLLNSFYPKDINVKWKVDGVIQDTGIQESV |
| | kappa | TEQDKDSTYSLSSTLTMSSTEYLSHELYSCEITHKSLPSTLIKSFQRSECQRVD |
| 29 | Traditional Canine VL- | GQPKASPSVTLFPPSSEELGANKATLVCLISDFYPSGVTVAWKADGSPVTQGVETT |
| | lambda | KPSKQSNNKYAASSYLSLTPDKWKSHSSFSCLVTHEGSTVEKKVAPAECS |
| 30 | VH-heavy constant region | ASTTAPSVFPLAPSCGSTSGSTVALACLVSGYFPEPVTVSWNSGSLTSGVHTFPS |
| | with knob mutations at | VLQSSGLYSLSSMVTVPSSRWPSETFTCNVAHPASKTKVDKPVPKRENGRVPRPP |
| | T366W and S354C | DCPKCPAPEAAGGPSVFIFPPKPKDTLLIARTPEVTCVVVDLDPEDPEVQISWFVD |
| | | GKQMQLAKTQPREEQFNGTYRVVSVLPIGHQDWLKGKQFTCKVNNKALPSPIERT |
| | | ISKARGQAHQPSVYVLPPCREELSKNTVSLWCLIKDFFPPDIDVEWQSNGQQEPE |
| | | SKYRTTPPQLDEDGSYFLYSKLSVDKSRWQRGDTFICYVMHEALHNHYTQESLSH |
| | | SPGK |

Expression was performed by matrixing the three VLCH1 constructs and five VHkappa_Hole constructs, a total of 15 kappa CanBiMabs were expressed. Using the three VLCH1 constructs and 3 VHlambda_Hole constructs, a total of nine lambda CanBiMabs were expressed. The bispecific antibodies were expressed in a 1:1:1:1 ratio in Chinese hamster ovary (CHO) cells and purified using Protein A resin.

Concentration was measured using A280 on a NANODROP™ OneC instrument (THERMO SCIENTIFIC™). Transient yields were extremely low, and sodium dodecyl sulfate-polyacrylamide gel electrophoresis (SDS-PAGE) followed by Coomassie staining revealed that the major species was not a full IgG, migrating slightly above 100 kDa.

### Example 2. Structural Evaluation of Canine Constant Regions

Due to the lack of stability in the initial attempt, structural modeling was performed to understand the expected behavior of CanBiMab formats in bispecific antibody design. Two X-ray crystal structures of human constructs from the Protein Data Bank (PDB) were used to reference crossed (PDB Reference No. 4IML) and non-crossed states (PDB Reference No. 4IMK) (see, e.g., Fenn et al., PLoS ONE 8: e61953, 2013). 4IML presents a crossed light chain with VL domain and CH1 (VL-CH1) and crossed heavy chain with VH domain and Ckappa (VH-Ckappa) with a resolution of 2.93 Å. 4IMK captures the wild-type of 4IML with the same variable region, non-crossed structure with a resolution of 2.20 Å. To resolve missing density and properly minimize the references for comparable analysis, both structures underwent AlphaFold2 Multimer modeling and Rosetta Relax protocol as described below.

For experimental analysis, 15 CanBiMab constructs were generated using the three light chain variants and five heavy chain variants (SEQ ID NOs: 1-8). Each variant retained the chain's respective variable region (VL or VH) with different modifications to the crossed CH1 and Ckappa domains. Complexes were modeled using AlphaFold2 Multimer to generate an ensemble of 25 Alphafold2-ranked models per reference and construct (see, e.g., Evans et al., bioRxiv 2021.10.04.463034, 2021). Ensemble modeling allows for an expanded and diverse conformational sampling space for analysis. Models did not include the Fc or hinge region as the work focused on the Fab region. Each ensemble generated by AlphaFold2 Multimer was refined using the Rosetta Relax protocol. Rosetta Relax finds low-energy backbone and side-chain conformations near starting conformation through rounds of packing and minimizing (see, e.g., Conway et al., Protein Science 23: 47-55, 2014). Output relaxed models were given a total energy score in Rosetta energy units (REU). Top models (lowest REU) were selected for visualization.

To analyze the models, root mean square deviation (RMSD) was used for measuring structural fluctuation and flexibility in a protein structure. The per-residue Ca backbone RMSD was calculated using the Rosetta relaxed AlphaFold2 ensembles for each reference and construct using a sliding window approach. The averaged RMSDs were then mapped to the B-factor column of the top relaxed models for each residue for visualization in PyMOL (Schrödinger, LLC). Using PyMOL's putty mode, areas of structural fluctuation were visible, where thicker putty indicated a greater magnitude in RMSD and therefore higher variation, while thinner putty indicated a smaller magnitude in RMSD and lower variation. Areas of higher variation were indicative of potential areas to investigate as contributing to stability issues.

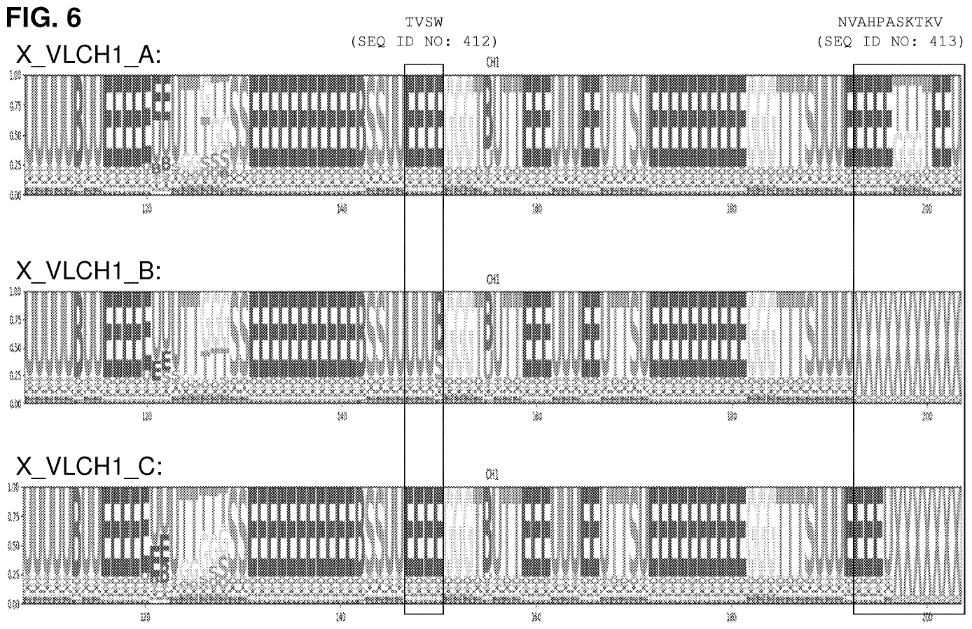

Significant changes in secondary structure due to crossing or different modifications would be indicative of resulting stability and expression challenges. To assess changes in secondary structure, logo plots were generated for each reference and construct ensemble to take advantage of the sampled conformational space. Using Biopython's DSSP module, secondary structure records were extracted from each model and transformed into a frequency for the logo plot generated with the Python logomaker package (Cock et al., Bioinformatics 25: 1422-1423, 2009; Tareen and Kinney, Bioinformatics 36: 2272-2274, 2020). Secondary structure features and their corresponding letter codes used for this analysis included: helix (H), isolated beta strands (B), beta sheets (E), 3-10 helix (G), pi helix (I), turn (T), bend (S), and unstructured (U). To handle truncations and resulting differences in sequence lengths, the letter X was used as a spacer for padding sequences in the final logo plots (FIG. 6).

In addition to modeling the canine sequences as CrossMabs, the non-crossed state was also analyzed. Modeling the non-crossed state of the canine Fab revealed that the canine sequence NVAHPASKTKVDKP (SEQ ID NO: 31) plays a critical role in completing the secondary structure of CH1.

Given the importance of understanding the secondary structure and conformation of the non-crossed state of the canine Fab, modeling was extended to include the hinge region and Fc domain to determine how the CH1 canine secondary structure might be affected. Models were generated in identical fashion as described above—Alphafold2 Multimer generated an ensemble of models which were then relaxed using Rosetta Relax protocol. Top models were used for visualization and inspection to see how secondary structure might have been modified.

Modeling showed that the longest sequence of CH1 had the best conservation of structure (see, e.g., FIG. 6). However, the protein was truncated before the end of a beta sheet and had the presence of an unsatisfied nitrogen and oxygen in V208. Therefore, extending the sequence further with the addition of aspartic acid (D) 209, lysine (K) 210, and proline (P) 211 (referred to as the DKP residues) would be needed to complete the beta sheet. Initial analysis revealed that the inclusion of the DKP residues to the canine CH1 sequence C-terminus was critical in maintaining secondary structure in the domain. To determine if variants required the extra canine sequence as a linker, Rosetta relaxed AlphaFold2 Multimer ensembles were generated with the longest light chain CH1 variant with the addition of the DKP residues at the C-terminus and the original five heavy chain variants. Models were visually inspected for destabilization or disruption to secondary structure.

Ending a protein on a proline was not preferred. Therefore, additional modeling was performed to determine if the domain could be further extended. Residues from the hinge region (e.g., VPKRENGRVPRPPDCPKCP (SEQ ID NO: 35)) were used to extend the C-terminus of the crossed CH1 light chain. C-terminal extensions included: V, VPK, VPKR (SEQ ID NO: 32), and VPKRE (SEQ ID NO: 33). Resulting variants were modeled using AlphaFold2 Multimer and refined with Rosetta Relax to generate structural ensembles for inspection. These additional models showed that adding in valine 212 from the hinge was helpful for hydrophobic packing and even going further in the hinge, shielding the core with the addition of the proline 213 and lysine 214, and may add additional stability for the construct.

As for the VHkappa_Hole chains, modeling showed that the kappa constant maintained secondary structure, and having the additional end terminal kappa sequence or heavy chain sequence after the last CH1 cysteine was not needed and may have unwanted flexibility.

### Example 3. Evaluation of Refined CH1-CL CanBiMabs

To test the modeling predictions, additional VLCH1 constructs were designed (see Table 5 below for sequences and FIG. 3 for alignments). The additional constructs included: one that adds the additional DKP residues to the X_VLC1_A construct from Table 3 (SEQ ID NO: 12), one that adds the DKP along with two glycine residues at the C-terminal to avoid ending on a proline (SEQ ID NO: 13), one that adds valine 212 from the hinge region after the DKP residues (SEQ ID NO: 14), and the last that adds valine 212, proline 213, and lysine 214 (SEQ ID NO: 15). These VLCH1 constructs were paired with the two VHkappa_Hole chains that had the least amount of additional sequence, X_VHkappa_Hole_Y (SEQ ID NO: 5), X_VHkappa_Hole_V (SEQ ID NO: 8), and with the VHlambda_Hole_Y chain (SEQ ID NO: 10).

TABLE 5
Additional canine VLCH1 constant region designs

| SEQ ID | | |
|---|---|---|
| NO: | Chain | Sequence |
| 12 | Constant Canine | SSASTTAPSVFPLAPSCGSTSGSTVALACLVSGYFPEPVTVSWNSGSLTSGVHTF |
| | X_VLCH1_D | PSVLQSSGLYSLSSMVTVPSSRWPSETFTCNVAHPASKTKVDKP |
| 13 | Constant Canine | SSASTTAPSVFPLAPSCGSTSGSTVALACLVSGYFPEPVTVSWNSGSLTSGVHTF |
| | X_VLCH1_E | PSVLQSSGLYSLSSMVTVPSSRWPSETFTCNVAHPASKTKVDKPGG |
| 14 | Constant Canine | SSASTTAPSVFPLAPSCGSTSGSTVALACLVSGYFPEPVTVSWNSGSLTSGVHTF |
| | X_VLCH1_F | PSVLQSSGLYSLSSMVTVPSSRWPSETFTCNVAHPASKTKVDKPV |
| 15 | Constant Canine | SSASTTAPSVFPLAPSCGSTSGSTVALACLVSGYFPEPVTVSWNSGSLTSGVHTF |
| | X_VLCH1_G | PSVLQSSGLYSLSSMVTVPSSRWPSETFTCNVAHPASKTKVDKPVPK |

The evaluation of CanBiMabs was performed using anti-NGF and anti-Activin RIIB variable regions. The four chains consisted of a CanBiMabs VLCH1, a CanBiMabs VHkappaHole (or VHlambda_Hole), a traditional VL-lambda (or VL-kappa), and a VH-heavy constant with knob mutations (SEQ ID NOs: 16-25; see Table 6 below). Using the four VLCH1 constructs and two VHkappa_Hole constructs, a total of eight kappa CanBiMabs were expressed. In initial experiments, only one VLCH1 construct (X_VLCH1_D) and one VHlambda_Hole construct were expressed. Bispecific antibodies were expressed in a 1:1:1:1 ratio in CHO cells and purified using Protein A resin.

TABLE 6
Exemplary CanBiMab heavy and light chain constructs

| SEQ ID | | |
|---|---|---|
| NO: | Chain | Sequence |
| 16 | X01-VHkappa- | EVQLVESGGDLVAPSQSLSITCTVSGMSLWSNSISWVRQPPGRGLEWLGTIWSQ |
| | Hole-Y | GGTTETDAVKGRFTISRDNAKNTVYLQMNSLRAEDTAMYYCAQIYYYDAEYLHWY |
| | | FDFWGQGTLVTVSSASDAQPAVYLFQPSPDQLHTGSASVVCLLNSFYPKDINVKW |
| | | KVDGVIQDTGIQESVTEQDKDSTYSLSSTLTMSSTEYLSHELYSCEITHKSLPSTLIK |
| | | SFQRSECVPKRENGRVPRPPDCPKCPAPEAAGGPSVFIFPPKPKDTLLIARTPEVT |
| | | CVVVDLDPEDPEVQISWFVDGKQMQLAKTQPREEQFNGTYRVVSVLPIGHQDWL |
| | | KGKQFTCKVNNKALPSPIERTISKARGQAHQPSVCVLPPSREELSKNTVSLSCAIKD |
| | | FFPPDIDVEWQSNGQQEPESKYRTTPPQLDEDGSYFLVSKLSVDKSRWQRGDTFI |
| | | CYVMHEALHNRFTQESLSHSPGK |
| 17 | X01-VHkappa- | EVQLVESGGDLVAPSQSLSITCTVSGMSLWSNSISWVRQPPGRGLEWLGTIWSQ |
| | Hole-V | GGTTETDAVKGRFTISRDNAKNTVYLQMNSLRAEDTAMYYCAQIYYYDAEYLHWY |
| | | FDFWGQGTLVTVSSASDAQPAVYLFQPSPDQLHTGSASVVCLLNSFYPKDINVKW |
| | | KVDGVIQDTGIQESVTEQDKDSTYSLSSTLTMSSTEYLSHELYSCEITHKSLPSTLIK |
| | | SFQRSECQRVDVPKRENGRVPRPPDCPKCPAPEAAGGPSVFIFPPKPKDTLLIART |
| | | PEVTCVVVDLDPEDPEVQISWFVDGKQMQLAKTQPREEQFNGTYRVVSVLPIGHQ |
| | | DWLKGKQFTCKVNNKALPSPIERTISKARGQAHQPSVCVLPPSREELSKNTVSLSC |
| | | AIKDFFPPDIDVEWQSNGQQEPESKYRTTPPQLDEDGSYFLVSKLSVDKSRWQRG |
| | | DTFICYVMHEALHNRFTQESLSHSPGK |
| 18 | X01_VLCH1_D | EIVMTQSPASLSASQEEKVTITCRASEGISKNLAWYQQKPGQAPKLLIHATSHLQTG |
| | | VPSRFSGSGSGTDFTLTISSLQPEDFATYYCQQGYQFPLTFGQGTKVEIKSSASTT |
| | | APSVFPLAPSCGSTSGSTVALACLVSGYFPEPVTVSWNSGSLTSGVHTFPSVLQS |
| | | SGLYSLSSMVTVPSSRWPSETFTCNVAHPASKTKVDKP |
| 19 | X01_VLCH1_E | EIVMTQSPASLSASQEEKVTITCRASEGISKNLAWYQQKPGQAPKLLIHATSHLQTG |
| | | VPSRFSGSGSGTDFTLTISSLQPEDFATYYCQQGYQFPLTFGQGTKVEIKSSASTT |
| | | APSVFPLAPSCGSTSGSTVALACLVSGYFPEPVTVSWNSGSLTSGVHTFPSVLQS |
| | | SGLYSLSSMVTVPSSRWPSETFTCNVAHPASKTKVDKPGG |
| 20 | X01_VLCH1_F | EIVMTQSPASLSASQEEKVTITCRASEGISKNLAWYQQKPGQAPKLLIHATSHLQTG |
| | | VPSRFSGSGSGTDFTLTISSLQPEDFATYYCQQGYQFPLTFGQGTKVEIKSSASTT |
| | | APSVFPLAPSCGSTSGSTVALACLVSGYFPEPVTVSWNSGSLTSGVHTFPSVLQS |
| | | SGLYSLSSMVTVPSSRWPSETFTCNVAHPASKTKVDKPV |
| 21 | X01_VLCH1_G | EIVMTQSPASLSASQEEKVTITCRASEGISKNLAWYQQKPGQAPKLLIHATSHLQTG |
| | | VPSRFSGSGSGTDFTLTISSLQPEDFATYYCQQGYQFPLTFGQGTKVEIKSSASTT |
| | | APSVFPLAPSCGSTSGSTVALACLVSGYFPEPVTVSWNSGSLTSGVHTFPSVLQS |
| | | SGLYSLSSMVTVPSSRWPSETFTCNVAHPASKTKVDKPVPK |
| 22 | 006_T28S_ | EVQLVQSGAEVKKPGASVKVSCKTSGYSFTSSYINWVRQAPGAGLDWMGTINPV |
| | Q65K Knob | SGSTSYAQKFKGRVTLTADTSTSTAYMELSSLRAGDIAVYYCARGGWFDYWGQG |
| | | TLVTVSSASTTAPSVFPLAPSCGSTSGSTVALACLVSGYFPEPVTVSWNSGSLTSG |
| | | VHTFPSVLQSSGLYSLSSMVTVPSSRWPSETFTCNVAHPASKTKVDKPVPKRENG |
| | | RVPRPPDCPKCPAPEAAGGPSVFIFPPKPKDTLLIARTPEVTCVVVDLDPEDPEVQI |
| | | SWFVDGKQMQLAKTQPREEQFNGTYRVVSVLPIGHQDWLKGKQFTCKVNNKALP |
| | | SPIERTISKARGQAHQPSVYVLPPCREELSKNTVSLWCLIKDFFPPDIDVEWQSNG |
| | | QQEPESKYRTTPPQLDEDGSYFLYSKLSVDKSRWQRGDTFICYVMHEALHNHYT |
| | | QESLSHSPGK |
| 23 | 006_VL | QSVLTQPASVSGSLGQRVTISCTGSSSDVGSYNYVNWYQQLPGTGPRTLIYGVSK |
| | | RPSGVPDRFSGSKSGSTATLTISGLQAEDEADYYCGTFAGGSYYGVFGGGTHLTV |
| | | LGQPKASPSVTLFPPSSEELGANKATLVCLISDFYPSGVTVAWKADGSPVTQGVET |
| | | TKPSKQSNNKYAASSYLSLTPDKWKSHSSFSCLVTHEGSTVEKKVAPAECS |
| 24 | X08-VHlambda- | EVQLVQSGAEVKKPGASVKVSCKTSGYSFTSSYINWVRQAPGAGLDWMGTINPV |
| | Hole-Y | SGSTSYAQKFKGRVTLTADTSTSTAYMELSSLRAGDIAVYYCARGGWFDYWGQG |
| | | TLVTVSSASKASPSVTLFPPSSEELGANKATLVCLISDFYPSGVTVAWKADGSPVT |
| | | QGVETTKPSKQSNNKYAASSYLSLTPDKWKSHSSFSCLVTHEGSTVEKKVAPAEC |
| | | VPKRENGRVPRPPDCPKCPAPEAAGGPSVFIFPPKPKDTLLIARTPEVTCVVVDLD |
| | | PEDPEVQISWFVDGKQMQLAKTQPREEQFNGTYRVVSVLPIGHQDWLKGKQFTC |
| | | KVNNKALPSPIERTISKARGQAHQPSVCVLPPSREELSKNTVSLSCAIKDFFPPDID |
| | | VEWQSNGQQEPESKYRTTPPQLDEDGSYFLVSKLSVDKSRWQRGDTFICYVMHE |
| | | ALHNRFTQESLSHSPGK |
| 25 | X08_VLCH1_D | QSVLTQPASVSGSLGQRVTISCTGSSSDVGSYNYVNWYQQLPGTGPRTLIYGVSK |
| | | RPSGVPDRFSGSKSGSTATLTISGLQAEDEADYYCGTFAGGSYYGVFGGGTHLTV |
| | | LSSASTTAPSVFPLAPSCGSTSGSTVALACLVSGYFPEPVTVSWNSGSLTSGVHTF |
| | | PSVLQSSGLYSLSSMVTVPSSRWPSETFTCNVAHPASKTKVDKP |

Concentration was measured using A280 on a NANODROP™ OneC instrument (THERMO SCIENTIFIC™). Transient yields showed improvement of production with VLCH1 constructs that contained hinge region extension sequence, showing that the addition of only residues D209-P211 was not sufficient to rescue protein stability. Expression was increased with the addition of valine (V) 212 and further increased with the addition of the proline (P) and lysine (K) at positions 213 and 214, respectively.

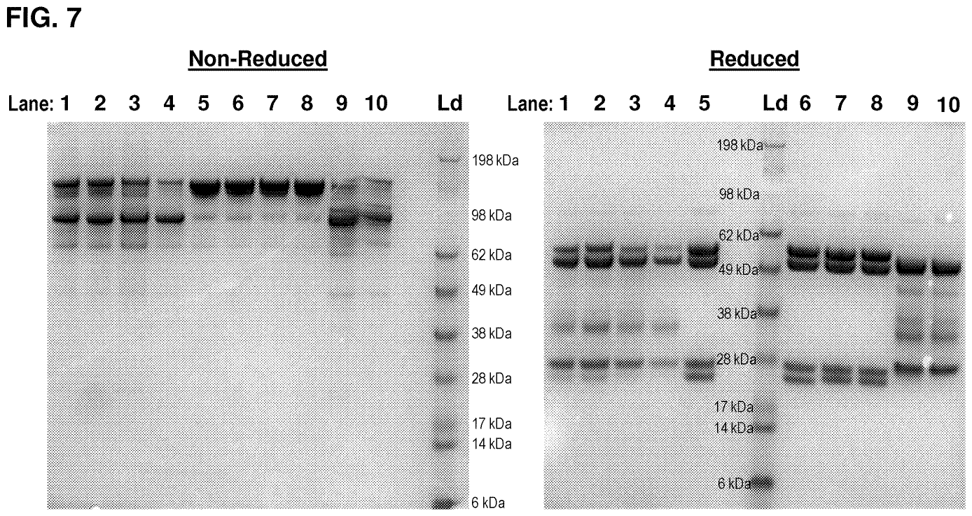

Non-reduced SDS-PAGE gels revealed that the constructs with the added V212 or V212-K214 had a prominent band of the expected migration of a full IgG (FIG. 7, non-reduced Lanes 5-8). The reduced SDS-PAGE showed that these constructs had similar band intensity for all four chains at the expected migration (FIG. 7, reduced lanes 5-8). The constructs that only had the addition of D209-P211 had many bands in the non-reduced SDS-PAGE with the most prominent band migrating around 100 kDa (FIG. 7, non-reduced lanes 1-4, 9, and 10). The four CanBiMabs with the correct size were further characterized.

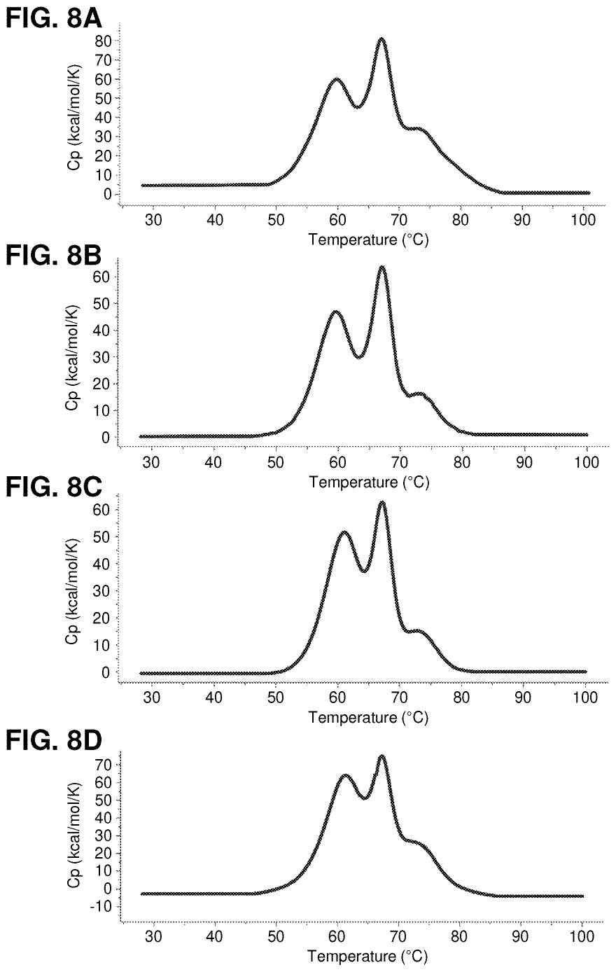

As an indicator of conformational stability, the melting temperature (Tₘ) of the protein samples were measured by differential scanning calorimetry (DSC) on a Malvern MicroCal PEAQ-DSC. A scan rate of 60° C./h was used over the temperature range of 25-100° C. Samples were diluted using a 20 mM sodium acetate pH 5.5, 136 mM sodium chloride buffer. The DSC thermograms are shown in FIGS. 8A-8D, and melting temperatures are summarized in Table 7 below. These data indicate that constructs with the addition of V212 had a Tₒₙₛₑₜ around 49° C. while those with V212-K214 had a slightly higher Tₒₙₛₑₜ and higher subsequent melting transitions.

TABLE 7
DSC results for CanBiMabs

| | Tₒₙₛₑₜ | Tₘ₁ | Tₘ₂ | Tₘ₃ |
|---|---|---|---|---|
| Protein Name | (° C.) | (° C.) | (° C.) | (° C.) |
| X_VLCH1_F:X_Vhkappa_Hole | 49.60 | 59.59 | 66.99 | 73.64 |
| Y: VL:VH | | | | |
| X_VLCH1_F:X_Vhkappa_Hole | 49.11 | 59.53 | 66.98 | 73.49 |
| V: VL:VH | | | | |
| X_VLCH1_G:X_Vhkappa_Hole | 51.02 | 60.92 | 67.18 | 73.30 |
| Y: VL:VH | | | | |
| X_VLCH1_G:X_Vhkappa_Hole | 50.17 | 61.26 | 68.00 | 72.53 |
| V: VL:VH | | | | |

Size exclusion was performed on an Agilent 1260 Infinity II using a MABPAC™ SEC-1 Size Exclusion (4×150 mm, 5μm) column to evaluate the presence and prevalence of high molecular weight species (HMWS) and low molecular weight species (LMWS). The mobile phase was 50 mM sodium phosphate, 0.3 M NaCl, pH 6.8, with an isocratic flow of 0.1 mL/min for 30 mins. There was no observed HMWS or LMWS in the four constructs evaluated.

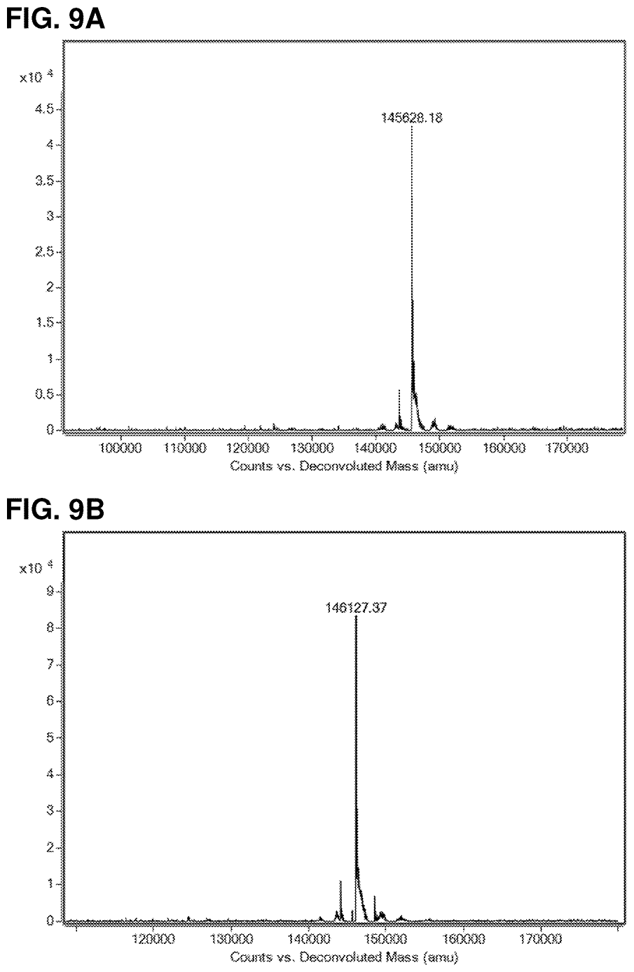

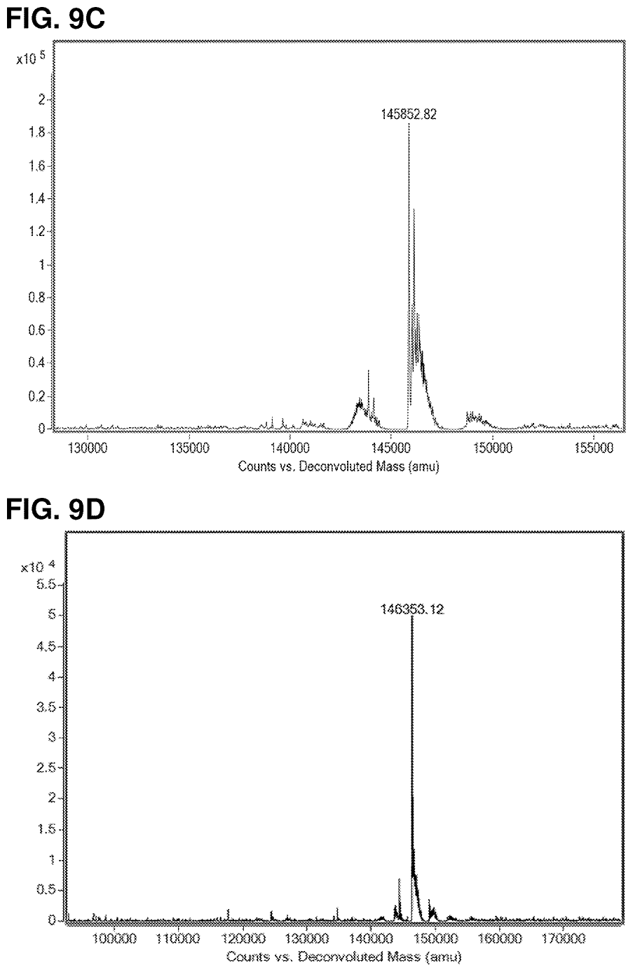

Chain pairing was assessed by intact mass using liquid chromatography mass spectrometry. Samples were deglycosylated with PNGase F and run either reduced or non-reduced over a PLRP-S(2.1 ×50 mm, 8 μm) column on an Agilent 1290 Infinity II uPLC followed by an Agilent 6530 Electrospray Ionization Quadruple Time-of-Flight Mass Spectrometer (ESI-QToF). The assignments of measured masses were determined using RioConfirm 10.0 software. Results are summarized in Table 8 below, and the chromatographic mass spectra profiles are shown in FIGS. 9A-9D for the non-reduced results.

TABLE 8
Reduced and non-reduced, deglycosylated intact mass results of CanBiMab constructs

| | | | | | Relative |
|---|---|---|---|---|---|
| | | Theoretical | Observed | | Percent |
| Sample Name | Chain | MW | MW | Assignment | Intensity |
| X_VLCH1_F:X_Vhkappa_Hole | VLCH1_F = | 21813.49 | 21814.95 | LC1 | 100 |
| Y: Vk:VH | LC1 | | | | |
| | VHkappa_Hole | 52278.77 | 52153.57 | HC1 − Lysine | 86.7 |
| | Y = HC1 | | 52315.27 | HC1 − Lysine + Glycation | 13.3 |
| | VHHC_knob = | 49199.56 | 49073.94 | HC2 − Lysine | 100 |
| | HC2 | | | | |
| | VL_lambdaLC = | 22635.08 | 22619.09 | LC2 + PyroQ | 100 |
| | LC2 | | | | |
| | Non-reduced | 145926.9 | 143635.27 | LC1 + (HC1 − Lysine) + (HC2 − | 9.0 |
| | (all 4 chains | | | Lysine) + (LC2 + PyroQ) + 16 | |
| | present) | | | Disulfide Bonds − 1994 Da | |
| | | | 145628.18 | LC1 + (HC1 − Lysine) + (HC2 − | 64.7 |
| | | | | Lysine) + (LC2 + PyroQ) + 16 | |
| | | | | Disulfide Bonds | |
| | | | 145787.16 | LC1 + (HC1 − Lysine + Glycation) + | 26.3 |
| | | | | (HC1 − Lysine) + (LC2 + PyroQ) + 16 | |
| | | | | Disulfide Bonds | |
| X_VLCH1_F:X_Vhkappa_Hole | VLCH1_F = | 21813.49 | 21814.73 | LC1 | 100 |
| V: Vk:VH | LC1 | | | | |
| | VHkappa_Hole | 52777.31 | 52651.84 | HC1 − Lysine | 86.2 |
| | V = HC1 | | 52813.31 | HC1 − Lysine + Glycation | 13.8 |
| | VHHC_knob = | 49199.56 | 49073.83 | HC2 − Lysine | 100 |
| | HC2 | | | | |
| | VL_lambdaLC = | 22635.08 | 22618.06 | LC2 + PyroQ | 100 |
| | LC2 | | | | |
| | Non-reduced | 146425.44 | 144129.89 | LC1 + (HC1 − Lysine) + (HC2 − | 8.9 |
| | (all 4 chains | | | Lysine) + (LC2 + PyroQ) + 16 | |
| | present) | | | Disulfide Bonds − 1997 Da | |
| | | | 146127.37 | LC1 + (HC1 − Lysine) + (HC2 − | 67.7 |
| | | | | Lysine) + (LC2 + PyroQ) + 16 | |
| | | | | Disulfide Bonds | |
| | | | 146288.89 | LC1 + (HC1 − Lysine + Glycation) + | 23.4 |
| | | | | (HC1 − Lysine) + (LC2 + PyroQ) + 16 | |
| | | | | Disulfide Bonds | |
| X_VLCH1_G:X_Vhkappa_Hole | VLCH1_G = | 22038.78 | 22039.75 | LC1 | 100 |
| Y: Vk:VH | LC1 | | | | |
| | VHkappa_Hole | 52278.77 | 52153.71 | HC1 − Lysine | 85.0 |
| | Y = HC1 | | 52316.65 | HC1 − Lysine + Glycation | 15.0 |
| | VHHC_knob = | 49199.56 | 49074.12 | HC2 − Lysine | 100 |
| | HC2 | | | | |
| | VL_lambdaLC = | 22635.08 | 22618.97 | LC2 + PyroQ | 100 |
| | LC2 | | | | |
| | Non-reduced | 146335.36 | 143855.77 | LC1 + (HC1 − Lysine) + (HC2 − | 7.5 |
| | (all 4 chains | | | Lysine) + (LC2 + PyroQ) + 16 | |
| | present) | | | Disulfide Bonds − 1997 Da | |
| | | | 145852.82 | LC1 + (HC1 − Lysine) + (HC2 − | 63.1 |
| | | | | Lysine) + (LC2 + PyroQ) + 16 | |
| | | | | Disulfide Bonds | |
| | | | 146015.04 | LC1 + (HC1 − Lysine + Glycation) + | 29.4 |
| | | | | (HC1 − Lysine) + (LC2 + PyroQ) + 16 | |
| | | | | Disulfide Bonds | |
| X_VLCH1_G:X_Vhkappa_Hole | VLCH1_G = | 22038.78 | 22039.90 | LC1 | 100 |
| V: Vk:VH | LC1 | | | | |
| | VHkappa_Hole | 52777.31 | 52651.95 | HC1 − Lysine | 86.7 |
| | V = HC1 | | 52813.36 | HC1 − Lysine + Glycation | 13.3 |
| | VHHC_knob = | 49199.56 | 49073.74 | HC2 − Lysine | 100 |
| | HC2 | | | | |
| | VL_lambdaLC = | 22635.08 | 22619.09 | LC2 + PyroQ | 100 |
| | LC2 | | | | |
| | Non-reduced | 146650.73 | 144359.50 | LC1 + (HC1 − Lysine) + (HC2 − | 8.9 |
| | (all 4 chains | | | Lysine) + (LC2 + PyroQ) + 16 | |
| | present) | | | Disulfide Bonds − 1994 Da | |
| | | | 146353.12 | LC1 + (HC1 − Lysine) + (HC2 − | 65.8 |
| | | | | Lysine) + (LC2 + PyroQ) + 16 | |
| | | | | Disulfide Bonds | |
| | | | 146517.02 | LC1 + (HC1 − Lysine + Glycation) + | 25.3 |
| | | | | (HC1 − Lysine) + (LC2 + PyroQ) + 16 | |
| | | | | Disulfide Bonds | |

PyroQ = pyroglutamic acid.

Non-reduced intact mass analysis confirmed the assembly of the CanBiMab constructs. The major species observed for all four constructs was “HC1+HC2+LC1+LC2” with full removal of all heavy chain C-terminal lysine residues and N-terminal cyclization on the lambda light chain. The next most abundant species also had all four constructs with the addition of glycation. Reduced intact mass concluded this glycation was located on the VHkappa_Hole arm, but it was unclear if this was in the variable or constant region. There was also a species of about 7.5-9% in each construct with a 2000 Da lower molecular weight. It was possible that this was a knob:knob construct. Consequently, the percentage of confirmed correctly paired CanBiMabs ranged for these four constructs from 90-92.5%. Since these constructs were only purified by affinity chromatography, it was expected that byproducts can be removed by polishing purification steps.

Affinity to the different targets of the bispecific constructs was determined by surface plasmon resonance (SPR) using a BIACORE™ T200 SPR system. The antibodies were captured using a CM5 Series S chip amine coupled with an anti-dog Fc antibody (Jackson ImmunoResearch). Antigen binding was then assessed at multiple concentrations starting at 100 nM of NGF or 100 nM of Activin RIIB using PBSP+ running buffer (Cytiva) with a flow rate of 30 μL/min. The length of the association time was 120 s, and the dissociation time was run for 600 s. The chip surface was regenerated with 10 mM glycine. Reference-subtracted sensorgrams were fitted to a 1:1 binding model using BIACORE™ T200 Evaluation software. The data is shown in Table 9 below.

TABLE 9
Affinity of CanBiMabs to both antigens

| | | | | | | Difference |
|---|---|---|---|---|---|---|
| Construct | Antigen | ka (1/Ms) | kd (1/s) | KD (M) | Rmax | in Rmax |
| Parental mAb (on CanBiMabs | NGF | 4.97e+5 | 1.41e−5 | 2.84e−11 | 45.95 | NA |
| side) | | | | | | |
| Parental mAb (on Fab side) | Activin | 4.52e+5 | 1.12e−4 | 2.49e−10 | 34.60 | NA |
| | RIIB | | | | | |
| X_VLCH1_F:X_Vhkappa_Hole | NGF | 5.55e+5 | 1.08e−5 | 1.97e−11 | 21.50 | 46.8% |
| Y: VL:VH | | | | | | |
| X_VLCH1_F:X_Vhkappa_Hole | Activin | 5.24e+5 | 1.28e−4 | 2.44e−10 | 15.15 | 43.8% |
| Y: VL:VH | RIIB | | | | | |
| X_VLCH1_F:X_Vhkappa_Hole | NGF | 5.40e+5 | 8.44e−6 | 1.56e−11 | 20.00 | 43.5% |
| V: VL:VH | | | | | | |
| X_VLCH1_F:X_Vhkappa_Hole | Activin | 5.40e+5 | 1.24e−4 | 2.30e−10 | 15.10 | 43.6% |
| V: VL:VH | RIIB | | | | | |
| X_VLCH1_G:X_Vhkappa_Hole | NGF | 5.63e+5 | 1.24e−5 | 2.16e−11 | 21.60 | 47.0% |
| Y: VL:VH | | | | | | |
| X_VLCH1_G:X_Vhkappa_Hole | Activin | 5.23e+5 | 1.20e−4 | 2.29e−10 | 14.90 | 43.1% |
| Y: VL:VH | RIIB | | | | | |
| X_VLCH1_G:X_Vhkappa_Hole | NGF | 5.71e+5 | 2.88e−5 | 5.05e−11 | 20.95 | 45.6% |
| V: VL:VH | | | | | | |
| X_VLCH1_G:X_Vhkappa_Hole | Activin | 5.34e+5 | 1.21e−4 | 2.29e−10 | 13.65 | 39.4% |
| V: VL:VH | RIIB | | | | | |

The binding of all CanBiMabs was confirmed to both antigens and had similar affinity as the parental antibodies. Due to the monovalency of the bispecific constructs, the Rmax was expected to be about half that of the Rmax of the parental monoclonal antibody (mAb). The difference between the observed CanBiMab Rmax and the parental Rmax ranged from 39.4% to 47.0%. These data agreed that most of the bispecific antibodies were binding their respective antigens, suggesting that they were correctly paired.

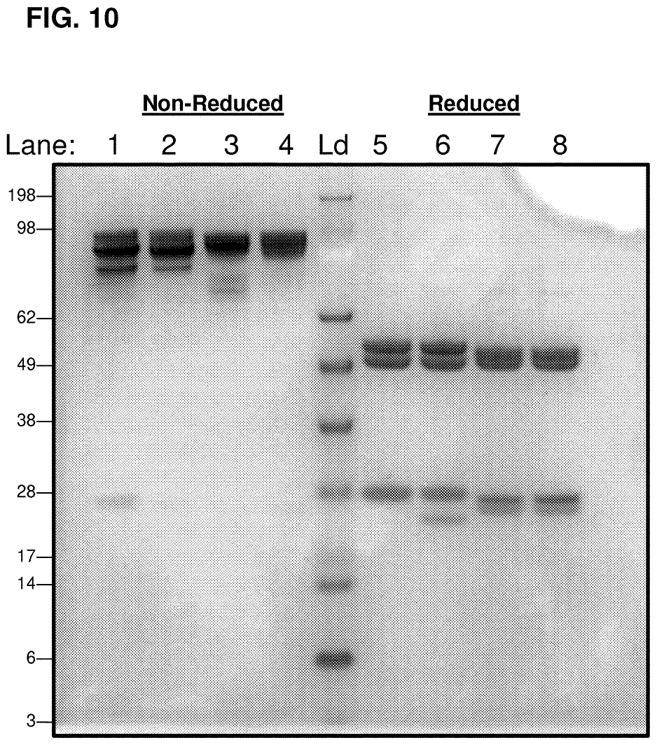

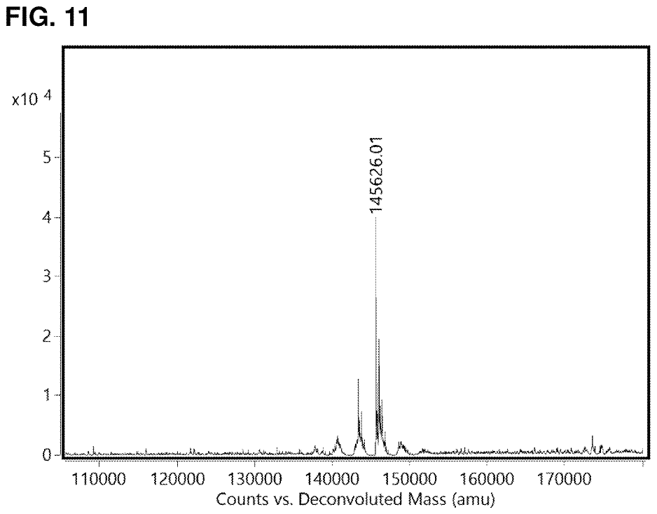

After observing the rescued expression and correct pairing with X_VLC1_G (SEQ ID NO: 15), constructs with lambda variable were designed on the CanBiMab arm and paired with VHlambda_Hole_Y (SEQ ID NO: 10) to confirm if the extended CH1 sequence would yield the same results (SEQ ID NOs: 406-409, Table 10). Constructs were made with the Protein A binding site (H435/Y436) and without the C-terminal lysine of the heavy chain since it was observed to be clipped in the intact mass results in Table 8. Expression was performed as previously described and concentration confirmed that expression was rescued compared to the initial attempts. Non-reduced SDS-PAGE showed the protein migrated at the expected size, and non-reduced, deglycosylated intact mass confirmed correctly paired CanBiMabs (FIG. 10, Lanes 4 and 8; FIG. 11; and Table 11).

TABLE 10
Exemplary CanBiMab heavy and light chain constructs

| SEQ ID | | |
|---|---|---|
| NO: | Chain | Sequence |
| 406 | X16-VLCH1_G | QSALTQPSSVSGTLGQTVTISCDGSSSDVGSYNYVNWYQQFPGTSPKLLIYGVSR |
| | | RPSGIPARFSGSKSGNTASLTISGLQAEDEADYYCGTFAGGSYYGVFGGGTHLTV |
| | | LSSASTTAPSVFPLAPSCGSTSGSTVALACLVSGYFPEPVTVSWNSGSLTSGVHTF |
| | | PSVLQSSGLYSLSSMVTVPSSRWPSETFTCNVAHPASKTKVDKPVPK |
| 407 | X16-Vhlambda- | EVQLVESGGDLVKPGGSLRLSCVASGYSFTSTYIQWVRQAPGKGLQWMGTINPV |
| | Hole-Y | SGSTLYADAVKGRFTISRDNARNTVYLQMNSLRAEDTAVYYCARSGWFDYWGQG |
| | | TLVTVSSASKASPSVTLFPPSSEELGANKATLVCLISDFYPSGVTVAWKADGSPVT |
| | | QGVETTKPSKQSNNKYAASSYLSLTPDKWKSHSSFSCLVTHEGSTVEKKVAPAEC |
| | | VPKRENGRVPRPPDCPKCPAPEAAGGPSVFIFPPKPKDTLLIARTPEVTCVVVDLD |
| | | PEDPEVQISWFVDGKQMQLAKTQPREEQFNGTYRVVSVLPIGHQDWLKGKQFTC |
| | | KVNNKALPSPIERTISKARGQAHQPSVCVLPPSREELSKNTVSLSCAIKDFFPPDID |
| | | VEWQSNGQQEPESKYRTTPPQLDEDGSYFLVSKLSVDKSRWQRGDTFICYVMHE |
| | | ALHNHYTQESLSHSPG |
| 408 | 1178_VH_knob | EVQLVESGGDLVKPGGSLRLSCVASGFTFSNYDMAWVRQAPGKGLQWVASMSP |
| | | GGGSIYYRDAVKGRFTISRDNARNTVYLQMNSLRAEDTAVYYCAREGELGPFDYW |
| | | GQGTLVTVSSASTTAPSVFPLAPSCGSTSGSTVALACLVSGYFPEPVTVSWNSGS |
| | | LTSGVHTFPSVLQSSGLYSLSSMVTVPSSRWPSETFTCNVAHPASKTKVDKPVPK |
| | | RENGRVPRPPDCPKCPAPEAAGGPSVFIFPPKPKDTLLIARTPEVTCVVVDLDPED |
| | | PEVQISWFVDGKQMQLAKTQPREEQFNGTYRVVSVLPIGHQDWLKGKQFTCKVN |
| | | NKALPSPIERTISKARGQAHQPSVYVLPPCREELSKNTVSLWCLIKDFFPPDIDVEW |
| | | QSNGQQEPESKYRTTPPQLDEDGSYFLYSKLSVDKSRWQRGDTFICYVMHEALH |
| | | NHYTQESLSHSPG |
| 409 | 1178-VL | EIVMTQSPASLSLSQEEKVTITCRASQSVGINVDWYQQKPGQAPKLLIYGASNRHT |
| | | GVPSRFSGSGSGTDFTFTISSLEPEDVAVYYCLQYGSIPWTFGQGTKLEIKRNDAQ |
| | | PAVYLFQPSPDQLHTGSASVVCLLNSFYPKDINVKWKVDGVIQDTGIQESVTEQDK |
| | | DSTYSLSSTLTMSSTEYLSHELYSCEITHKSLPSTLIKSFQRSECQRVD |

TABLE 11
Non-reduced, deglycosylated intact mass results
of the exemplary CanBiMab construct

| | | | | | Relative |
|---|---|---|---|---|---|
| | Theoretical | Observed | Major | | Percent |
| Chain | MW | MW | Species | Observed Species | Intensity |
| Non-reduced | 145637.64 | 145626.01 | Yes | (CrossMab VL + PyroQ) + | 76.53% |
| (all 4 chains | | | | VL + CrossMab VH + | |
| present) | | | | VH_Knob | |
| | | 145787.79 | No | (CrossMab VL + PyroQ) + | 23.47% |
| | | | | VL + CrossMab VH + | |
| | | | | VH_Knob + Glycation | |

PyroQ = pyroglutamic acid.

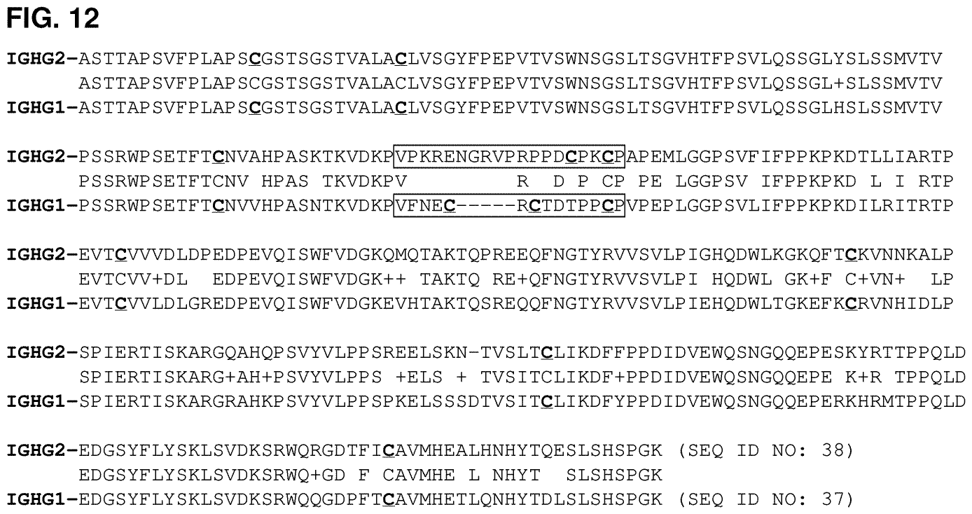

The above work was performed with the canine IgGB isotype (also known as canine IGHG2). For other isotypes, the same structural concepts are applicable with some variation. As shown in FIG. 12, the canine IGHG1 hinge has an additional cysteine compared to canine IGHG2 (VFNECRCTDTPPCP (SEQ ID NO: 34) vs VPKRENGRVPRPPDCPKCP (SEQ ID NO: 35)). Therefore, the CH1 domain in VLCH1 constructs for IGHG1 can extend to the first cysteine in the hinge, which includes amino acids VFNEC (SEQ ID NO: 36).

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and that this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

1. A bispecific antibody comprising:
(a) a first antigen-binding moiety that binds to a first antigen, wherein the first antigen-binding moiety comprises:
(i) a first light chain comprising a first light chain variable region (VL) and a first light chain constant domain (CL); and
(ii) a first heavy chain comprising a first heavy chain variable region (VH), a first heavy chain constant domain 1 (CH1), and a first Fc subunit; and
(b) a second antigen-binding moiety that binds to a second antigen, wherein the second antigen-binding moiety comprises:
(i) a second light chain comprising a second VL and a second CH1; and
(ii) a second heavy chain comprising a second VH, a second CL, and a second Fc subunit,
and
wherein the first CH1 and the second CH1 are derived from a CH1 of a canine IgG, the first CL and the second CL are derived from a CL of a canine IgG, and the first Fc subunit and the second Fc subunit are derived from an Fc subunit of a canine IgG.

2. The bispecific antibody of claim 1, wherein:
I) the second CH1 comprises a sequence having at least 80% identity to:
(a) a continuous sequence of amino acid residues within SEQ ID NO: 37 that comprises at least amino acids 1-83 or amino acids 1-97 of SEQ ID NO: 37;
(b) a continuous sequence of amino acid residues within SEQ ID NO: 38 that comprises at least amino acids 1-83 or amino acids 1-102 of SEQ ID NO: 38;
(c) a continuous sequence of amino acid residues within SEQ ID NO: 39 that comprises at least amino acids 1-X of SEQ ID NO: 39; or
(d) a continuous sequence of amino acid residues within SEQ ID NO: 40 that comprises at least amino acids 1-X of SEQ ID NO: 40; and/or
II) the second CL comprises a sequence having at least 80% identity to:
(a) a continuous sequence of amino acid residues within SEQ ID NO: 28 that comprises at least amino acids 3-106 of SEQ ID NO: 28; or
(b) a continuous sequence of amino acid residues within SEQ ID NO: 29 that comprises at least amino acids 4-105 of SEQ ID NO: 29.

3-5. (canceled)

6. The bispecific antibody of claim 1, wherein
I) the second CH1 further comprises;
(a) serine-serine linker at the N-terminus; and/or
(b) one or more glycine residues at the C-terminus; and/or
II) the second CL further comprises:
(a) an alanine-serine linker at the N-terminus;
(b) one or more continuous or discontinuous amino acid residues within the sequence of SEQ ID NO: 31; and/or
(c) the sequence of SEQ ID NO: 31.

7-12. (canceled)

13. The bispecific antibody of claim 1, wherein the first Fc subunit and the second Fc subunit comprise complementary dimerization selectivity modules that promote dimerization between the first Fc subunit and the second Fc subunit.

14. The bispecific antibody of claim 13, wherein;
I) the first Fc subunit and the second Fc subunit each comprises a protuberance or a cavity, and wherein:
(a) the first Fc subunit comprises a protuberance, and the second Fc subunit comprises a cavity; or
(b) the first Fc subunit comprises a cavity, and the second Fc subunit comprises a protuberance;
or
II) the first Fc subunit comprises a first charged region and the second Fc subunit comprises a second charged region, wherein the first charged region forms a charge pair with the second charged region, and wherein:
(a) the first charged region comprises a basic amino acid residue, and the second charged region comprises an acidic amino acid residue; or
(b) the first charged region comprises an acidic amino acid residue, and the second charged region comprises a basic amino acid residue.

15. (canceled)

16. The bispecific antibody of claim 14, wherein the first Fc subunit and the second Fc subunit comprise amino acid substitutions:
I) (a) 354C and 366W in the first Fc subunit; and 349C, 366S, 368A, and 407V in the second Fc subunit;
(b) 349C, 366S, 368A, and 407V in the first Fc subunit; and 354C and 366W in the second Fc subunit;
(c) 366W in the first Fc subunit; and 366S, 368A, and 407V in the second Fc subunit;
(d) 366S, 368A, and 407V in the first Fc subunit; and 366W in the second Fc subunit;
(e) 364H and 405A in the first Fc subunit; and 349T and 394F in the second Fc subunit;
(f) 349T and 394F in the first Fc subunit; and 364H and 405A in the second Fc subunit;
(g) 405L in the first Fc subunit; and 409R in the second Fc subunit;
(h) 409R in the first Fc subunit; and 405L in the second Fc subunit;
(i) 366L, 392L, and 394W in the first Fc subunit; and 351Y, 405A, and 407V in the second Fc subunit;
(j) 351Y, 405A, and 407V in the first Fc subunit; and 366L, 392L, and 394W in the second Fc subunit;
(k) 360E and 409W in the first Fc subunit; and 347R, 399V, and 405T in the second Fc subunit;
(l) 347R, 399V, and 405T in the first Fc subunit; and 360E and 409W in the second Fc subunit;
(m) 349C, 360E, and 409W in the first Fc subunit; and 347R, 354C, 399V, and 405T in the second Fc subunit;
(n) 347R, 354C, 399V, and 405T in the first Fc subunit; and 349C, 360E, and 409W in the second Fc subunit;
(o) 370E and 409W in the first Fc subunit; and 357N, 399V, and 405T in the second Fc subunit;
(p) 357N, 399V, and 405T in the first Fc subunit; and 370E and 409W in the second Fc subunit;
(q) 360D, 399M, and 407A in the first Fc subunit; and 345R, 347R, 366V, and 409V in the second Fc subunit;
(r) 345R, 347R, 366V, and 409V in the first Fc subunit; and 360D, 399M, and 407A in the second Fc subunit;
(s) 349S, 366M, 370Y, and 409V in the first Fc subunit; and 356G, 357D, 364Q, and 407A in the second Fc subunit;
(t) 356G, 357D, 364Q, and 407A in the first Fc subunit; and 349S, 366M, 370Y, and 409V in the second Fc subunit;
(u) 368D and 370S in the first Fc subunit; and 356Q and 364K in the second Fc subunit;
(v) 356Q and 364K in the first Fc subunit; and 368D and 370S in the second Fc subunit;
(w) 366Y in the first Fc subunit; and 366S, 368A, and 407T in the second Fc subunit; or
(x) 366S, 368A, and 407T in the first Fc subunit; and 366Y in the second Fc subunit,
wherein the amino acid positions are based on EU numbering; or
II) (a) K409D in the first Fc subunit; and D399K in the second Fc subunit;
(b) D399K in the first Fc subunit; and K409D in the second Fc subunit;
(c) K390D and K409D in the first Fc subunit; and E356K and D399K in the second Fc subunit;
(d) E356K and D399K in the first Fc subunit; and K390D and K409D in the second Fc subunit;
(e) K390D and K409D in the first Fc subunit; and E357K and D399K in the second Fc subunit;
(f) E357K and D399K in the first Fc subunit; and K390D and K409D in the second Fc subunit;
(a) K370D and K409D in the first Fc subunit; and E357K and D399K in the second Fc subunit;
(h) E357K and D399K in the first Fc subunit; and K370D and K409D in the second Fc subunit;
(i) R392D and K409D in the first Fc subunit; and E356K and D399K in the second Fc subunit;
(i) E356K and D399K in the first Fc subunit; and R392D and K409D in the second Fc subunit;
(k) L351 D and L368E in the first Fc subunit; and L351K and T366K in the second Fc subunit; or
(l) L351K and T366K in the first Fc subunit; and L351 D and L368E in the second Fc subunit,
wherein the amino acid positions are based on EU numbering.

17-22. (canceled)

23. The bispecific antibody of claim 1, wherein:
(a) the first Fc subunit or the second Fc subunit comprises the amino acid substitutions 435R and 436F;
(b) the first Fc subunit and the second Fc subunit comprise at least one of the following amino acid substitutions:
(i) 252Y and, optionally, at least one amino acid substitution selected from the group consisting of 251 D or 251 E; 285N or 285D; 286D, 286Y, 286F, 286L, or 286W; 307Q; 308P; 315D; 426L, 426H, 426F, or 426Y; 430A or 430K; 433K; 435Y; and 436H;
(ii) 252M and, optionally, at least one amino acid substitution selected from the group consisting of 251 D or 251 E; 256D or 256F; 285N or 285D; 286D, 286Y, 286F, 286L, or 286W; 307Q; 308P; 315D; 426L, 426H, 426F, or 426Y; 430A or 430K; 433K; 435Y; and 436H;
(iii) 434H and, optionally, at least one amino acid substitution selected from the group consisting of 286Y, 286F, 286L, or 286W; and 426Y, 426F, 426L, or 426W;
(iv) 434R;
(v) 426Y and, optionally, at least one amino acid substitution selected from the group consisting of 286F, 286W, 286L, or 286Y; 312P; 434R; and 436H;
(vi) 426H and, optionally, at least one amino acid substitution selected from the group consisting of 286F, 286W, 286L, or 286Y; 312P; 434R; and 436H;
(vii) 426F and, optionally, at least one amino acid substitution selected from the group consisting of 286F, 286W, 286L, or 286Y; 312P; 434R; and 426H; or
(viii) 434R and, optionally, at least one amino acid substitution selected from the group consisting of 286L; 286Y; 312P; and 436H;
(c) the first Fc subunit and the second Fc subunit comprise the amino acid substitutions 426Y and 286L;
(d) the first Fc subunit and the second Fc subunit comprise the amino acid substitutions 234A and 235A; and/or
(e) the first Fc subunit comprises the amino acid substitutions 234A, 235A, 286L, 354C, 366W, and 426Y and the second Fc subunit comprises the amino acid substitutions 234A, 235A, 286L, 349C, 366S, 368A, 407V, 426Y, 435R, and 436F,
wherein the amino acid positions are based on EU numbering.

24-28. (canceled)

29. The bispecific antibody of claim 1, wherein the first CH1 is linked to the first Fc subunit by a first hinge region and the second CL is linked to the second Fc subunit by a second hinge region, and wherein:
(a) the first CH1 is linked at the C-terminus to the N-terminus of the first hinge region, and the first hinge region is linked at the C-terminus to the N-terminus of the first Fc subunit; and/or
(b) the second CL is linked at the C-terminus to the N-terminus of the second hinge region, and the second hinge region is linked at the C-terminus to the N-terminus of the second Fc subunit.

30. (canceled)

31. The bispecific antibody of claim 29, wherein the first hinge region and the second hinge region comprise a sequence of:
(a)
(SEQ ID NO: 35)
VPKRENGRVPRPPDCPKCP;
(b)
(SEQ ID NO: 34)
VFNECRCTDTPPCP;
(c)
(SEQ ID NO: 45)
PKRENGRVPRPPDCPKCP;
(d)
(SEQ ID NO: 46)
FNECRCTDTPPCPVPEP;
(e)
(SEQ ID NO: 47)
PKRENGRVPRPPDCPKCPAPEM;
(f)
(SEQ ID NO: 48)
AKECECKCNCNNCPCPGCGL;
(g)
(SEQ ID NO: 49)
PKESTCKCISPCPVPES;
(h)
(SEQ ID NO: 50)
PKESTCKCIPPCPVPES;
(i)
(SEQ ID NO: 51)
KTDHPPGPKPCDCPKCP;
or
(j)
(SEQ ID NO: 52)
KTASTIESKTGEGPKCP.

32-43. (canceled)

44. The bispecific antibody of claim 1, wherein:
(a) the first Fc subunit and the second Fc subunit comprise a sequence having at least 80% identity to any one of SEQ ID NOs: 41-44;
(b) the first light chain comprises a sequence having at least 80% identity to SEQ ID NO: 28 or 29;
(c) the first heavy chain comprises a sequence having at least 80% identity to SEQ ID NO: 30;
(d) the second light chain comprises a sequence having at least 80% identity to any one of SEQ ID NOs: 1-3 and 12-15; and/or
(e) the second heavy chain comprises a sequence having at least 80% identity to any one of SEQ ID NOs: 4-11.

45-46. (canceled)

47. The bispecific antibody of claim 1, wherein:
(a) the first Fc subunit and the second Fc subunit are variants of wild type canine IgGA, IgGB, IgGC, or IgGD Fc;
(b) the first antigen and the second antigen are each independently selected from the group consisting of nerve growth factor (NGF), Activin receptor type IIA (RIIA), Activin receptor type IIB (RIIB), TrKA, ADAMTS, IL-1, IL-2, IL-4, IL-4R, angiotensin type 1 (AT1) receptor, angiotensin type 2 (AT2) receptor, IL-5, IL-12, IL-13, IL-31, IL-31R, IL-33, CD3, CD20, CD47, CD52, and a complement system component, and/or
(c) the first VL and the second VL each comprises a set of complementarity-determining region-light chain 1 (CDR-L1), CDR-L2, and CDR-L3 sequences independently selected from Table 2, and the first VH and the second VH each comprises a set of complementarity-determining region-heavy chain 1 (CDR-H1), CDR-H2, and CDR-H3 sequences independently selected from Table 2.

48. (canceled)

49. An antibody or antigen-binding fragment thereof capable of specifically binding to nerve growth factor (NGF), wherein the antibody or antigen-binding fragment thereof comprises a complementarity-determining region-heavy chain 1 (CDR-H1), CDR-H2, and CDR-H3, and a complementarity-determining region-heavy chain 1 (CDR-L1), CDR-L2, and CDR-L3, wherein:
(a) the CDR-H1 comprises the amino acid sequence of SEQ ID NO: 55 or a variant thereof with up to two conservative amino acid substitutions;
(b) the CDR-H2 comprises the amino acid sequence of SEQ ID NO: 56 or a variant thereof with up to two conservative amino acid substitutions;
(c) the CDR-H3 comprises the amino acid sequence of SEQ ID NO: 57 or a variant thereof with up to two conservative amino acid substitutions;
(d) the CDR-L1 comprises the amino acid sequence of SEQ ID NO: 58 or a variant thereof with up to two conservative amino acid substitutions;
(e) the CDR-L2 comprises the amino acid sequence ATS or a variant thereof with up to two conservative amino acid substitutions; and
(f) the CDR-L3 comprises the amino acid sequence of SEQ ID NO: 59 or a variant thereof with up to two conservative amino acid substitutions.

50. (canceled)

51. The antibody or antigen-binding fragment thereof of claim 49, wherein the antibody or antigen-binding fragment thereof comprises;
(a) a heavy chain variable region (VH) comprising a sequence having at least 80% identity to SEQ ID NO: 60, and a light chain variable region (VL) comprising a sequence having at least 80% identity to SEQ ID NO: 61;
(b) a canine Fc domain variant comprising a sequence having at least 80% identity to any one of SEQ ID NOs: 41-44; and/or
(c) a heavy chain comprising a sequence having at least 80% identity to SEQ ID NO: 26 and a light chain comprising a sequence having at least 80% identity to SEQ ID NO: 27.

52. (canceled)

53. The antibody or antigen-binding fragment thereof of claim 49, wherein:
(a) the antibody or antigen-binding fragment thereof is a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, an antibody-like protein scaffold, a Fv fragment, a Fab fragment, a F(ab′)2 molecule, or a tandem scFv (taFv); and/or
(b) the antibody or antigen-binding fragment thereof is of IgG, IqA, IqM, IqD, or IE isotype.

54-57. (canceled)

58. The antibody or antigen-binding fragment thereof of claim 49, wherein the antibody or antigen-binding fragment thereof comprises a canine Fc domain variant comprising:
(a) at least one of the following amino acid substitutions:
(i) 252Y and, optionally, at least one amino acid substitution selected from the group consisting of 251 D or 251 E; 285N or 285D; 286D, 286Y, 286F, 286L, or 286W; 307Q; 308P; 315D; 426L, 426H, 426F, or 426Y; 430A or 430K; 433K; 435Y; and 436H;
(ii) 252M and, optionally, at least one amino acid substitution selected from the group consisting of 251 D or 251 E; 256D or 256F; 285N or 285D; 286D, 286Y, 286F, 286L, or 286W; 307Q; 308P; 315D; 426L, 426H, 426F, or 426Y; 430A or 430K; 433K; 435Y; and 436H;
(iii) 434H and, optionally, at least one amino acid substitution selected from the group consisting of 286Y, 286F, 286L, or 286W; and 426Y, 426F, 426L, or 426W;
(iv) 434R;
(v) 426Y and, optionally, at least one amino acid substitution selected from the group consisting of 286F, 286W, 286L, or 286Y; 312P; 434R; and 436H;
(vi) 426H and, optionally, at least one amino acid substitution selected from the group consisting of 286F, 286W, 286L, or 286Y; 312P; 434R; and 436H;
(vii) 426F and, optionally, at least one amino acid substitution selected from the group consisting of 286F, 286W, 286L, or 286Y; 312P; 434R; and 426H; or
(viii) 434R and, optionally, at least one amino acid substitution selected from the group consisting of 286L; 286Y; 312P; and 436H;
(b) the amino acid substitutions 426Y and 286L; and/or
(c) the amino acid substitutions 234A and 235A,
wherein the amino acid positions are based on EU numbering.

59-64. (canceled)

65. A pharmaceutical composition comprising (i) the bispecific antibody of claim 1 and (ii) a pharmaceutically acceptable excipient.

66. (canceled)

67. A nucleic acid encoding the bispecific antibody of claim 1.

68-69. (canceled)

70. An isolated host cell comprising the nucleic acid of claim 67 or an expression vector comprising the nucleic acid.

71. A method of making a bispecific antibody, the method comprising:
(a) providing the nucleic acid of claim 67; and
(b) expressing the nucleic acid or the vector in a host cell culture, thereby producing the bispecific antibody, or the antibody or antigen-binding fragment thereof; and
(c) collecting the bispecific antibody or the antibody or antigen-binding fragment thereof produced in (b) from the host cell culture.

72. (canceled)

73. A method of treating or preventing a canine disease or disorder in a dog in need thereof, the method comprising administering an effective amount of the pharmaceutical composition of claim 65 to the dog, wherein the canine disease or disorder is:
(i) an allergic disease, a chronic pain, an acute pain, a skeletal or musculoskeletal disease, a metabolic disease, an inflammatory disease, an autoimmune disease, an endocrine disease, a gastrointestinal disease, a cardiovascular disease, a renal disease, a fertility related disorder, an infectious disease, or a cancer; or
(ii) atopic dermatitis, allergic dermatitis, osteoarthritic pain, muscular atrophy, diabetes, arthritis, anemia, or obesity.

74-80. (canceled)
